(19) United States
(12) Patent Application Publication
(10) Pub. No.: US 2020/0061076 A1
(43) Pub. Date: Feb. 27, 2020

(54) METHODS OF TREATING LEUKODYSTROPHIES

(71) Applicants: Ramot at Tel-Aviv University Ltd., Tel-Aviv (IL); Bar-llan University, Ramat-Gan (IL)

(72) Inventors: Orna ELROY-STEIN, Tel-Aviv (IL); Hanoch SENDEROWITZ, Tel-Aviv (IL); Andrea ATZMON, Netanya (IL)

(73) Assignees: Ramot at Tel-Aviv University Ltd., Tel-Aviv (IL); Bar-llan University, Ramat-Gan (IL)

(21) Appl. No.: 16/612,452

(22) PCT Filed: May 10, 2018
(86) PCT No.: PCT/IL2018/050516

§ 371 Date: Nov. 11, 2019

Related U.S. Application Data
(60) Provisional application No. 62/504,585, filed on May 11, 2017.

(51) Int. Cl. A61K 31/5377; A61K 31/426; A61K 31/53; A61K 31/15; A61K 31/121; A61K 31/4184; A61K 31/496; A61K 31/5375; A61K 31/495; A61K 31/505; A61K 31/18; A61P 25/28

(52) U.S. Cl. CPC A61K 31/5377; A61K 31/426; A61K 31/53; A61K 31/15; A61K 31/121; A61P 25/28; A61K 31/496; A61K 31/5375; A61K 31/495; A61K 31/505; A61K 31/18; A61K 31/4184

(57) ABSTRACT

Methods of treating leukodystrophy are provided. Accordingly there is provided a method of treating leukodystrophy in a subject, the method comprising administering to the subject a therapeutically effective amount of an agent capable of up-regulating activity and/or expression of a component participating in a Sigma-1 Receptor (Sig-1R) signaling pathway. Also provided are agents and methods of up-regulating activity of Sig-1R in a cell and treating a disease that can benefit from up-regulating activity of Sig-1R. Also provided are agents and methods of modulating activity of sonic hedgehog (SHH) in a cell and treating a disease that can benefit from modulating activity of SHH.

### FIELD AND BACKGROUND OF THE INVENTION

The present invention, in some embodiments thereof, relates to methods of treating leukodystrophies.

Leukodystrophies are a group of rare genetic disorders that affect the central nervous system (CNS) by disrupting the growth or maintenance of the myelin sheath, which insulates nerve cells.

Vanishing White Matter (VWM) disease, also termed Childhood Ataxia with CNS Hypomyelination (CACH), is a leukodystrophy characterized by progressive loss of white matter in both hemispheres of the brain. The consequent axonal degeneration results in progressive impairments of neurologic functions, leading to complete paralysis and early death. An important feature of this orphan disease is the deterioration of clinical symptoms upon exposure to physiological and environmental stressors. The disease is caused by mutations in any of the five genes encoding the eIF2B subunits (Leegwater et al. 2001), wherein most VWM mutations decrease the enzymatic activity of eIF2B. Depending on the severity of the mutations, the disease onset and clinical symptoms refer to congenital, classical and adult forms. The congenital form is extremely rare; the classical form refers to disease onset at early childhood and death around late teens, with <1000 known patients in the world; and the adult form which refers to a mild version seems increasingly common. There is no treatment for VWM which is manifested by progressive deterioration of the clinical symptoms.

Hypo-active eIF2B renders cells hypersensitive to ER-stress (Kantor et al. 2005, Kantor et al. 2008). Accordingly, VWM pathology involves a defective ER-stress response (van der Voorn et al. 2005, van Kollenburg et al. 2006). ER-stress can lead to mitochondrial unfolded protein response (UPRmt) via physiological and mechanistic connection between these two compartments (Giorgi et al. 2009), while mitochondrial defects could generate ER-stress by decreasing correct protein folding in the ER due to depleted ATP levels (Ryan & Hoogenraad 2007).

A homozygous mouse model, Eif2b5^{R132H/R132H}, has ˜20% decrease in brain eIF2B GEF activity leading to mild impairment of motor functions with involvement of white matter deficits. This mouse model provided fundamental insights related to the etiology of the VWM disease, including delayed postnatal brain development, abnormal glial cell abundance, increased abundance of demyelinated axons and axons unsheathed with split and damaged myelin, failure to overcome cuprizone-induced demyelination, poor astrogliosis and impaired cerebral inflammatory response upon insults (Geva et al. 2010, Cabilly et al. 2012). At the molecular level, delayed waves of gene expression, unbalanced expression of UPR-related genes, dysregulation of mitochondrial functions and oxidative respiration deficiency were indicated (Marom et al. 2011; Gat-Viks et al. 2015; Raini et al., 2017).

Additional background art includes:
Ishikawa M. et al. (2010) Journal of Receptor, Ligand and Channel Research, 3: 25-36;
Su et al. (2016) Trends in Pharmacological Sciences, 37(4): 262-278;
Li et al. (2016) Journal of Experimental & Clinical Cancer Research 35: 184; International Patent Application Publication Nos: WO 2010141932, WO 2013022738, WO 2014205229 and WO2013112859; and
US Patent Application Publication Nos: US 20160237455 and US 20140088140.

### SUMMARY OF THE INVENTION

According to an aspect of some embodiments of the present invention there is provided a method of treating leukodystrophy in a subject in need thereof, the method comprising administering to the subject a therapeutically effective amount of an agent capable of up-regulating activity and/or expression of a component participating in a Sigma-1 Receptor (Sig-1R) signaling pathway, thereby treating the leukodystrophy in the subject.

According to an aspect of some embodiments of the present invention there is provided an agent capable of up-regulating activity and/or expression of a component participating in a Sig-1R signaling pathway, for use in the treatment of leukodystrophy.

According to some embodiments of the invention, the component is selected from the group consisting of Sig-1R, CYC1, PHB, SLC25A11, SLC25A39, VSAC2, BiP, IRE1, RAC1, VDAC2, IP3R, Ankyrin, Insig, Emerin, RanBP2, ELMOD, UP1, C14orf1, CYP51A1, CFTR, EIF5A, GANAB, HSD17B1, 2HSPA5, NSDHL, RDH11, RPN2, SC4MOL, SEC61A2, SQLE, SURF4, TM7SF2, NACA2, PDZD11, RAF1, RPS27A, SEC61A2, TM7SF2, UBA52, UBC, XPO1, XPOT, CLN3, LBR, NUP205 and RAE1.

According to some embodiments of the invention, the component is Sig-1R.

According to some embodiments of the invention, the up-regulating is manifested by increased mitochondrial respiration and decreased ER stress as compared to same in the absence of the agent.

According to some embodiments of the invention, the agent is a small molecule.

According to some embodiments of the invention, the small molecule is a compound represented by Formula I:

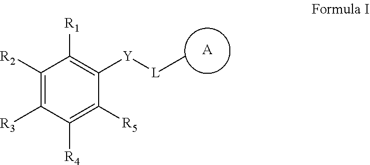

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;

Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety.

According to some embodiments of the invention, the A is a nitrogen-containing heterocyclic moiety.

According to some embodiments of the invention, the A is a morpholine.

According to some embodiments of the invention, the A is a piperazine.

According to some embodiments of the invention, the L is a saturated hydrocarbon chain of 2 to 10 carbon atoms in length.

According to some embodiments of the invention, the L is an alkylene chain of 2 to 10 carbon atoms in length.

According to some embodiments of the invention, the L is a hydrocarbon chain of 4 to 6 carbon atoms in length.

According to some embodiments of the invention, the L is an alkylene chain of 4 to 6 carbon atoms in length.

According to some embodiments of the invention, the Y is O.

According to some embodiments of the invention, the at least one R₁-R₅ is an alkyl.

According to some embodiments of the invention, the R₁ and/or R₂ is an alkyl.

According to some embodiments of the invention, the alkyl is methyl.

According to some embodiments of the invention, the at least one of R₁-R₅ is halo.

According to some embodiments of the invention, the R₄ and/or R₅ is halo.

According to some embodiments of the invention, the at least of R₁-R₅ is alkoxy.

According to some embodiments of the invention, the R₂ and/or R₄ is alkoxy.

According to some embodiments of the invention, the alkoxy is methoxy.

According to some embodiments of the invention, the small molecule is selected from the group consisting of 4-[5-(3-methylphenoxy)pentyl]morpholine, 4-[5-(3,5-dimethylphenoxy)pentyl]morpholine, 4-[5-(3,4-dimethylphenoxy)pentyl]morpholine, 4-[6-(3-methylphenoxy)hexyl]morpholine, 4-[4-(3-methylphenoxy)butyl]morpholine, 4-[4-(3,4-dimethylphenoxy)butyl]morpholine, 4-[5-(3-methoxyphenoxy)pentyl]morpholine, 4-[5-(3-chlorophenoxy)pentyl]morpholine and 1-[5-(2-fluorophenoxy)pentyl]-4-methylpiperazine.

According to some embodiments of the invention, the small molecule is selected from the group consisting of 4-[5-(3-methylphenoxy)pentyl]morpholine, Pre-084, pridopidine, dextromethorphan, SA4503, pentazocine, SKF-10047, 3-ppp, Fluvoxamine, Igmesine, Pregnenolone-S, DHEA-S, Donepezil, PPBP, Clorgyline, Fluoxetine, Imipramine, Sertaline, Carbetapentane, Dimemorfan, Amantadine, Memantine, Cocaine, BD 737, 4-IBP, OPC-14523, Anavex 2-73, Amitriptyline, L-687,384, Dimethyltryptamine, Methylphenylpiracetam and SOMCL-668.

According to some embodiments of the invention, the small molecule is 4-[5-(3-methylphenoxy)pentyl]morpholine.

According to some embodiments of the invention, the small molecule is Anavex 2-73.

According to some embodiments of the invention, the small molecule is Pre-084.

According to some embodiments of the invention, the small molecule is pridopidine.

According to some embodiments of the invention, the agent is a peptide.

According to some embodiments of the invention, the agent is an antibody.

According to an aspect of some embodiments of the present invention there is provided a method of up-regulating activity of Sigma-1 Receptor (Sig-1R) in a cell, the method comprising:

(a) contacting the cell with 4-[5-(3-methylphenoxy)pentyl]morpholine, thereby activating the Sig-1R; and

(b) analyzing activity of Sig-1R signaling pathway.

According to an aspect of some embodiments of the present invention there is provided a method of up-regulating activity of Sigma-1 Receptor (Sig-1R) in a cell, the method comprising:

(a) contacting the cell with a compound represented by Formula I:

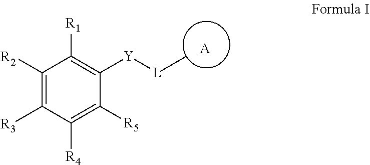

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;

Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety,

thereby activating the Sig-1R; and

(b) analyzing activity of Sig-1R signaling pathway.

According to an aspect of some embodiments of the present invention there is provided a method of modulating activity of sonic hedgehog (SHH) signaling pathway in a cell, the method comprising:

(a) contacting the cell with a compound selected from the group consisting of 1-allyl-2-(3,4,5-trimethoxyphenyl)-1H-benzimidazole, 1-(2-fluorophenyl)-4-(phenylacetyl)piperazine) and 1-allyl-2-(2-phenylvinyl)-1H-benzimidazole; and

(b) analyzing activity of SHH signaling pathway.

According to some embodiments of the invention, the modulating is down-regulating.

According to some embodiments of the invention, the compound is selected from the group consisting of 1-allyl-2-(3,4,5-trimethoxyphenyl)-1H-benzimidazole and 1-(2-fluorophenyl)-4-(phenylacetyl)piperazine.

According to some embodiments of the invention, the modulating is up-regulating.

According to some embodiments of the invention, the compound is 1-allyl-2-(2-phenylvinyl)-1H-benzimidazole.

According to an aspect of some embodiments of the present invention there is provided a method of modulating activity of 11β-hydroxysteroid dehydrogenase type 1 (11β-HSD1) in a cell, the method comprising:

(a) contacting the cell with 2-[(2,6-dimethyl-1-piperidinyl)carbonyl]-7-methyl-5-phenylpyrazolo[1,5-a]pyrimidine; and

(b) analyzing activity of 11β-HSD1.

According to some embodiments of the invention, the modulating is down-regulating.

According to some embodiments of the invention, the contacting is effected in-vivo.

According to some embodiments of the invention, the contacting is effected in-vitro or ex-vivo.

According to an aspect of some embodiments of the present invention there is provided a method of treating a disease that can benefit from up-regulating activity of Sigma-1 Receptor (Sig-1R), the method comprising administering to the subject a therapeutically effective amount of 4-[5-(3-methylphenoxy)pentyl]morpholine, thereby treating the disease in the subject.

According to an aspect of some embodiments of the present invention there is provided a method of treating a disease that can benefit from up-regulating activity of Sigma-1 Receptor (Sig-1R), the method comprising administering to the subject a therapeutically effective amount of a compound represented by Formula I:

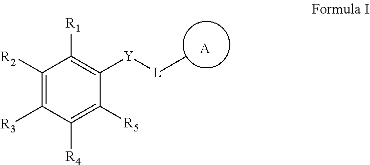

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide; Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety,

thereby treating the disease in the subject.

According to an aspect of some embodiments of the present invention there is provided a 4-[5-(3-methylphenoxy)pentyl]morpholine for use in the treatment of a disease that can benefit from up-regulating activity of Sigma-1 Receptor (Sig-1R).

According to an aspect of some embodiments of the present invention there is provided a compound represented by Formula I:

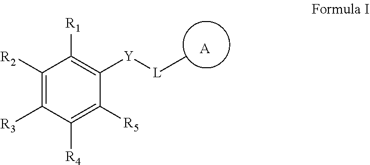

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;

Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety,

for use in the treatment of a disease that can benefit from up-regulating activity of Sigma-1 Receptor (Sig-1R).

According to some embodiments of the invention, the compound is selected from the group consisting of 4-[5-(3-methylphenoxy)pentyl]morpholine, 4-[5-(3,5-dimethylphenoxy)pentyl]morpholine, 4-[5-(3,4-dimethylphenoxy)pentyl]morpholine, 4-[6-(3-methylphenoxy)hexyl]morpholine, 4-[4-(3-methylphenoxy)butyl]morpholine, 4-[4-(3,4-dimethylphenoxy)butyl]morpholine, 4-[5-(3-methoxyphenoxy)pentyl]morpholine, 4-[5-(3-chlorophenoxy)pentyl]morpholine and 1-[5-(2-fluorophenoxy)pentyl]-4-methylpiperazine.

According to an aspect of some embodiments of the present invention there is provided a method of treating a disease that can benefit from down-regulating activity of sonic hedgehog (SHH) signaling pathway, the method comprising administering to the subject a therapeutically effective amount of a compound selected from the group consisting of 1-allyl-2-(3,4,5-trimethoxyphenyl)-1H-benzimidazole and 1-(2-fluorophenyl)-4-(phenylacetyl)piperazine, thereby treating the disease in the subject.

According to an aspect of some embodiments of the present invention there is provided a compound selected from the group consisting of 1-allyl-2-(3,4,5-trimethoxyphenyl)-1H-benzimidazole and 1-(2-fluorophenyl)-4-(phenylacetyl)piperazine for use in the treatment of a disease that can benefit from down-regulating activity of sonic hedgehog (SHH) signaling pathway.

According to an aspect of some embodiments of the present invention there is provided a method of treating a disease that can benefit from up-regulating activity of sonic hedgehog (SHH) signaling pathway, the method comprising administering to the subject a therapeutically effective amount of 1-allyl-2-(2-phenylvinyl)-1H-benzimidazole, thereby treating the disease in the subject.

According to an aspect of some embodiments of the present invention there is provided a 1-allyl-2-(2-phenylvinyl)-1H-benzimidazole for use in the treatment of a disease that can benefit from up-regulating activity of sonic hedgehog (SHH) signaling pathway.

According to some embodiments of the invention, the disease is selected from the group consisting of skeletal muscle regeneration following injury and brain recovery following ischemic stroke.

According to some embodiments of the invention, the disease is associated with mitochondrial dysfunction, oxidative stress and/or ER stress.

According to some embodiments of the invention, the disease is associated with mitochondrial dysfunction and/or ER stress.

According to an aspect of some embodiments of the present invention there is provided a method of treating a disease associated with mitochondrial dysfunction, oxidative stress and/or ER stress, the method comprising administering to the subject a therapeutically effective amount of a compound selected from the group consisting of 5-benzyl-2-[(2-chlorophenyl)imino]-1,3-thiazolidin-4-one, 5-butyl-3-{[2-(4-morpholinyl)ethyl]thio}-5H-[1,2,4]triazino[5,6-b]indole, 2-phenyl-N′-({5-[3-(trifluoromethyl)phenyl]-2-furyl}methylene)acetohydrazide, 1-{3-[(4-chlorobenzyl)oxy]phenyl}ethanone, 1-allyl-2-(3,4,5-trimethoxyphenyl)-1H-benzimidazole, 7-(difluoromethyl)-N-[2-(4-morpholinyl)ethyl]-5-phenylpyrazolo[1,5-a]pyrimidine-3-carboxamide, 1-phenyl-4-[4-(2-thienylcarbonyl)-1-piperazinyl]phthalazine, 4-[5-(3-methylphenoxy)pentyl]morpholine, 1-(2-fluorophenyl)-4-(phenylacetyl)piperazine, 2-{[2-oxo-2-(1-piperidinyl)ethyl]thio}-4-phenyl-6-(trifluoromethyl)pyrimidine, N-[2-(phenylthio)cyclohexyl]benzenesulfonamide, 1-{[3-(benzyloxy)phenyl]carbonothioyl}-4-methylpiperazine, 5-phenyl-N-(2-thienylmethyl)-7-(trifluoromethyl)pyrazolo[1,5-a]pyrimidine-2-carboxamide, 2-(2,3-dihydro-9H-imidazo[1,2-a]benzimidazol-9-yl)-1-(4-hydroxyphenyl)ethanone hydrobromide, 2-[(2,5-dimethoxyphenyl)diazenyl]-1-methyl-1H-benzimidazole, 2-[(2,6-dimethyl-1-piperidinyl)carbonyl]-7-methyl-5-phenylpyrazolo[ 1,5-a]pyrimidine, 2-(4-morpholinylmethyl)-1-(1-naphthylmethyl)-1H-benzimidazole, 5-isopropyl-N-methyl-3-phenylpyrazolo[ 1,5-a]pyrimidin-7-amine, 4-[5-(3,5-dimethylphenoxy)pentyl]morpholine, 4-[5-(3,4-dimethylphenoxy)pentyl]morpholine, 4-[6-(3-methylphenoxy)hexyl]morpholine, 4-[4-(3-methylphenoxy)butyl]morpholine, 4-[4-(3,4-dimethylphenoxy)butyl]morpholine, 4-[5-(3-methoxyphenoxy)pentyl]morpholine, 4-[5-(3-chlorophenoxy)pentyl]morpholine and 1-[5-(2-fluorophenoxy)pentyl]-4-methylpiperazine, thereby treating the disease in the subject.

According to an aspect of some embodiments of the present invention there is provided a compound selected from the group consisting of from the group consisting of 5-benzyl-2-[(2-chlorophenyl)imino]-1,3-thiazolidin-4-one, 5-butyl-3-{[2-(4-morpholinyl)ethyl]thio}-5H-[1,2,4]triazino[5,6-b]indole, 2-phenyl-N′-({5-[3-(trifluoromethyl)phenyl]-2-furyl}methylene)acetohydrazide, 1-{3-[(4-chlorobenzyl)oxy]phenyl}ethanone, 1-allyl-2-(3,4,5-trimethoxyphenyl)-1H-benzimidazole, 7-(difluoromethyl)-N-[2-(4-morpholinyl)ethyl]-5-phenylpyrazolo[ 1,5-a]pyrimidine-3-carboxamide, 1-phenyl-4-[4-(2-thienylcarbonyl)-1-piperazinyl]phthalazine, 4-[5-(3-methylphenoxy)pentyl]morpholine, 1-(2-fluorophenyl)-4-(phenylacetyl)piperazine, 2-{[2-oxo-2-(1-piperidinyl)ethyl]thio}-4-phenyl-6-(trifluoromethyl)pyrimidine, N-[2-(phenylthio)cyclohexyl]benzenesulfonamide, 1-{[3-(benzyloxy)phenyl]carbonothioyl}-4-methylpiperazine, 5-phenyl-N-(2-thienylmethyl)-7-(trifluoromethyl)pyrazolo[1,5-a]pyrimidine-2-carboxamide, 2-(2,3-dihydro-9H-imidazo[ 1,2-a]benzimidazol-9-yl)-1-(4-hydroxyphenyl)ethanone hydrobromide, 2-[(2,5-dimethoxyphenyl)diazenyl]-1-methyl-1H-benzimidazole, 2-[(2,6-dimethyl-1-piperidinyl)carbonyl]-7-methyl-5-phenylpyrazolo[1,5-a]pyrimidine, 2-(4-morpholinylmethyl)-1-(1-naphthylmethyl)-1H-benzimidazole, 5-isopropyl-N-methyl-3-phenylpyrazolo[1,5-a]pyrimidin-7-amine, 4-[5-(3,5-dimethylphenoxy)pentyl]morpholine, 4-[5-(3,4-dimethylphenoxy)pentyl]morpholine, 4-[6-(3-methylphenoxy)hexyl]morpholine, 4-[4-(3-methylphenoxy)butyl]morpholine, 4-[4-(3,4-dimethylphenoxy)butyl]morpholine, 4-[5-(3-methoxyphenoxy)pentyl]morpholine, 4-[5-(3-chlorophenoxy)pentyl]morpholine and 1-[5-(2-fluorophenoxy)pentyl]-4-methylpiperazine for use in the treatment of a disease associated with mitochondrial dysfunction, oxidative stress and/or ER stress.

According to some embodiments of the invention, the disease is selected from the group consisting of leukodystrophy, multiple sclerosis, cancer, OXPHOS diseases, lactic acidosis and stroke-like episodes (MELAS), myoclonus epilepsy with ragged red fibers (MERRF), deafness-dystonia syndrome (DDP), Parkinson disease, diabetes mellitus and sensorineural hearing impairment.

According to some embodiments of the invention, the cancer is selected from the group consisting of lung cancer, stomach cancer, esophagus cancer, pancreas cancer, prostate cancer, breast cancer, liver cancer, brain cancer, medulloblastoma, Basal cell carcinoma (BCC), cancer stem cells, rhabdomyosarcomas, glioma, multiple myeloma and chronic myelogenous leukemia (CML).

According to some embodiments of the invention, the disease is leukodystrophy.

According to some embodiments of the invention, the leukodystrophy is selected from the group consisting of vanishing white matter (VWM) disease, Krabbe disease, Metachromatic leukodystrophy, Pelizaeus-Merzbacher disease, Canavan disease, Adrenoleukodystrophy, Adrenomyeloneuropathy, Alexander disease, Cerebrotendineous xanthomatosis and Refsum disease.

According to some embodiments of the invention, the leukodystrophy is vanishing white matter (VWM) disease.

According to an aspect of some embodiments of the present invention there is provided a method of identifying an agent for the treatment of a disease associated with mitochondrial dysfunction, oxidative stress and/or ER stress, the method comprising determining a level of reactive oxygen species (ROS) in cells having an eIF2B deficiency following contacting with a test agent, wherein a decrease in the level of the ROS, as compared to same in the absence of the test agent, indicates efficiency of the test agent for the treatment of the disease.

According to some embodiments of the invention, the method comprising determining survival of the cells following the contacting, wherein no statistically significant change in survival of the cells following said contacting as compared to survival in the absence of the test agent, indicates efficiency of the test agent for the treatment of the disease.

According to some embodiments of the invention, the cells comprise fibroblasts or astrocytes.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows western blot photographs demonstrating reduced expression of Sig-1R in primary fibroblasts (MEFs) and primary astrocytes isolated from eIF2B5^{R132H/R132H} (Mut) mice as compared to same isolated from wild type C57BL (WT) mice. GAPDH served as a loading control; and following densitometry, Sig-1R/GAPDH ratio was calculated and set as 1 for the respective WT cells. Shown are representative blots (top) and calculated S1R/GAPDH ratio ±SEM of 3-8 independent experiments (bottom).

FIG. 2 is a bar graph demonstrating the effect of Sigma-1 receptor (Sig-1R) binders on mitochondrial content. DNA extracted from WT and Mut MEFs that were treated or not for 6 hours with 0.1 μM, 1 μM or 10 μM of p8G5 (blue), Pre084 (known SIG-1R agonist, purple), pridopidine (known SIG-1R agonists, green) or NE-100 (known SIG-1R antagonist, yellow) was subjected to qPCR analysis of mitochondrial 12S rRNA and nuclear 18S rRNA genes. Shown is relative quantity (RQ) normalized to untreated WT ±SEM of 2-4 independent experiments. * p<0.01.

FIG. 3 is a bar graph demonstrating that p8G5 reduced the levels of reactive oxygen species (ROS) in Mut MEFs. Mut MEFs were treated or untreated with 10 μM p8G5 for 24 hours, washed and stained with Hoechst, CFSE (fluorescent cell tracer) and the ROS detector CellROX for 30 minutes, followed by image acquisition using In-Cell-Analyzer-2000. Shown is average CellROX integrated intensity per cell relative to untreated WT MEFs ±SEM of 3 independent experiments.

FIG. 4 is a bar graph demonstrating no cellular toxicity induced by Sig-1R binders, as determined by survival of Mut MEFs. Mut MEFs were treated with the indicated concentrations of the indicated Sig-1R binders for 24 hours, washed and stained with Crystal Violet. Shown is absorbance at 570 nm normalized (%) to non-treated cells ±SEM of 2-5 independent experiments. * p=0.0003.

FIGS. 5A-B demonstrate the effect of Sigma-1 receptor (Sig-1R) binders on mitochondrial membrane potential, as determined by TMRE staining. Mut MEFs were treated or untreated with the indicated concentrations of the indicated Sig-1R binders for 6 hours, stained by TMRE for 30 minutes and analyzed by flow cytometry. Shown are flow cytometry histograms and bar graphs representing % mean TMRE fluorescence intensity relative to non-treated WT cells of a representative experiment.

FIG. 6 is a bar graph demonstrating the effect of Sigma-1 receptor (Sig-1R) binders on mitochondrial membrane potential, as determined by JC1 fluorescent dye. Mut MEFs were treated or not for 6 hours with the indicated concentrations of p8G5 (blue), pre084 (purple), or NE-100 (yellow), washed and stained with Hoechst and JC1 for 30 minutes followed by image acquisition using In-Cell-Analyzer-2000. Shown is the % of red/green JC1 integrated intensity per cell relative to that in untreated WT cells of 2-4 independent experiments.

FIGS. 7A-B are bar graphs demonstrating the effect of Sig-1R binders on oxidative phosphorylation per cell. Mut MEFs were treated or not for 6 hours with the indicated concentrations of the indicated Sig-1R binders. Oxygen consumption was measured in a XF96 Extracellular Flux Analyzer using the Seahorse XF Cell Mito Stress Test Kit. The Basal respiration (FIG. 7A) and ATP-linked respiration (FIG. 7B) data were normalized to biomass as determined by Crystal Violet staining. Shown is average % pmoles oxygen consumed per minute per biomass relative to non-treated WT MEFs ±SEM of 2-4 independent experiments. * p<0.03.

FIGS. 8A-C are bar graphs demonstrating the effect of Sig-1R binders on oxidative phosphorylation per mitochondrial content. Mut MEFs were treated or not for 6 hours with the indicated concentrations of the indicated Sig-1R binders. Oxygen consumption was measured in a XF96 Extracellular Flux Analyzer using the Seahorse XF Cell Mito Stress Test Kit. The Basal respiration (FIG. 8A), ATP-linked respiration (FIG. 8B) and maximal respiration (FIG. 8C) data was normalized to mtDNA content as detailed in FIG. 2. Shown is average % pmoles oxygen consumed per minute per mtDNA content relative to non-treated WT MEFs ±SEM of 2-4 independent experiments. * p<0.04.

FIG. 9 is a bar graph demonstrating the effect of Sig-1R binders on MEFs survival under Tunicamycin-induced ER stress conditions. Mut MEFs were treated or not for 24 hours with the indicated concentrations of the indicated Sig-1R binders in the presence of 0.1 μg/ml Tunicamycin (Tun) during the last 22 hours. Following, the cells were washed, fixated and stained with Crystal Violet (CV) followed by measurement of absorbance at 570 nm. Shown is average % of CV staining relative to cells not treated with tunicamycin nor Sig-1R binder ±SEM of 3 independent experiments. *p<0.03.

FIG. 10 demonstrates the effect of p8G5 on MEFs survival under Tunicamycin-induced ER stress conditions. WT and Mut MEFs were treated or not for 24 hours with 20 μM p8G5 in the presence of the indicated concentrations of tunicamycin (Tun) during the last 22 hours. Following, the cells were washed, fixated and stained with Crystal Violet and absorbance at 570 nm was measured. Data is presented relative to cells of the corresponding genotype not treated with p8G5 nor Tunicamycin. Shown is average % of CV staining ±SEM of 3 independent experiments.

FIGS. 11A-C demonstrate that the compounds p9E6, p8C10 and p8G7 modulate the Sonic hedgehog (SHH) pathway. FIGS. 11A-B demonstrate Firefly/Renilla luciferase expression in Shh-lightII cells treated with the indicated compounds at the indicated concentrations in the absence (FIG. 11A) or presence (FIG. 11B) of the SHH activator SAG at 50 nM for 24 hours. Following lysis, luminescence was measured using the Dual Luciferase assay kit (Promega) followed by Firefly/Renilla luciferase ratio calculation for each treatment. Shown is average Firefly/Renilla luciferase activity ±SD of 3 independent experiments. *p<0.04. FIG. 11C is a bar graph demonstrating SHH activity in WT and Mut brains, as determined by Gli1 mRNA levels. RNA was extracted from brains of P18 and P21 Mut and WT mice. cDNA was prepared by reverse transcription and subjected to qPCR using primers specific to Gli1 and GAPDH (SEQ ID NOs: 1-2 and 3-4, respectively). Relative quantity (RQ) was calculated by the ΔΔCt method. * p<0.02 [vs control (FIG. 11A) or vs SAG treated cells (FIG. 11B)].

FIG. 12 is a bar graph demonstrating the effect of cyclopamine on mitochondrial health. Mutant MEFs were incubated for 24 hours with 1 μM cyclopamine, a known SHH inhibitor. Following, the cells were washed, stained with Hoechst and JC-1 fluorescent dyes for 30 minutes, and subjected to image acquisition using In-Cell-Analyzer-2000. Shown is cellular content of damaged mitochondria (JC1 green channel, red bars), intact mitochondria (JC1 red channel, green bars) and damaged to intact mitochondria ratio (blue bars). Values of control (vehicle treated) cells was set to 100%.

FIG. 13 shows graphs demonstrating the effect of SHH agonist (SAG) and SHH antagonist (cyclopamine) on in-vitro differentiation of primary mouse OPC to mature oligodendrocytes. Primary OPC cultures isolated from newborn mice brains were incubated for 60 hours in differentiation medium (basal) in the presence of 20 nM SAG or 1 μM cyclopamine as indicated. Images were taken every 10 hours followed by analysis using IncuCyte ZOOM software (2016B).

FIGS. 14A-C demonstrate the effect of the 20 selected hits from the DIVERSet™-EXP library on ROS levels, survival and mitochondrial membrane potential. FIG. 14A is a bar graph demonstrating that hits H1-H20 reduced the levels of reactive oxygen species (ROS) in Mut MEFs. Mut MEFs were treated with the indicated compounds at 10 μM for 24 hours followed by staining with fluorogenic dyes and image-based single-cell analysis. Shown is average effect on ROS level from at least 3-independent experiments ±SEM. All values are statistically significant, p<0.02. The extreme SEM points of all values are below the 90% cut-off, therefore the actual average reduction is between 12% and 29%. FIG. 14B is a bar graph demonstrating survival of Mut MEFs following treatment with hits H1-H20. Mut MEFs were incubated or not for 24 hours with 10 μM of the indicated compound, washed and stained with Crystal Violet. Shown is average absorbance at 570 nm normalized (%) to non-treated cells of 3 independent experiments ±SEM, * p=0.0003. FIG. 14C is a bar graph demonstrating the effect of the indicated hits on mitochondrial membrane potential, as determined by JC1 fluorescent dye. Mut MEFs were incubated or not for 24 hours with 10 μM of the indicated compound, washed and stained with JC1 and Hoechst followed by image-based single-cell analysis. Values of untreated cells were set as 100%. Shown is average of at least 3 independent experiments for each parameter compared to its matching control ±SEM. * p<0.05. Putative targets are indicated.

FIG. 15 is a graph demonstrating the correlation between experimentally determined Ki values and calculated docking scores. Correlation (r²=0.7) between known Ki values and CDOCKER scores (kcal/mol) for known Sig-1R binders, as indicated. Higher CDOCKER values correspond to better binding energies.

FIGS. 16A-C show western blot photographs and quantitating graphs demonstrating reduced expression of Sig-1R per mitochondria of MEFs (FIG. 16A), primary astrocytes (FIG. 16B) and brains at postnatal age of P14 and P18 (FIG. 16C) isolated from Mut mice as compared to same isolated from wild type C57BL (WT) mice. SDHB served as mitochondria content marker. Shown are representative blots (top) and calculated S1R/SDHB ratio ±SEM of 3-6 independent experiments (bottom). *p=0.02 (MEFs), *p=0.01 (astrocytes), *p=0.048 (p14 brains), *p=0.003 (p18 brains).

FIGS. 17A-C demonstrate the effect of Sig-1R binders on mitochondria abundance and performance in astrocytes. FIG. 17A shows bar graphs demonstrating the effect of Sig-1R binders on mitochondrial content, as determined by qPCR analysis of mitochondrial 12S rRNA and nuclear 18S rRNA genes in WT (grey) and Mut (black) primary astrocytes incubated or not for 6 hours with the indicated compounds. Shown is average relative quantity (RQ) of 2-6 independent experiments normalized to their untreated controls ±SEM. FIGS. 17B-C show bar graphs demonstrating the effect of the Sig-1R binders on oxygen consumption (OCR) following incubation for 6 hours with the indicated compounds. Values of ATP-linked respiration (FIG. 17B) and maximal respiration (FIG. 17C) were analyzed and normalized to cell number. Shown is average normalized to untreated cells ±SEM of 6 replicates in a representative experiment from 4 independent experiments. *p<0.05.

FIG. 18 is a bar graph demonstrating the effect of Sig-1R binders on MEFs survival under Tunicamycin-induced ER stress conditions. Mut MEFs were treated or not for 24 hours with 10, 20 or 30 μM of the indicated Sig-1R binders in the presence of 50 ng/ml Tunicamycin (Tun) during the last 22 hours. Following, the cells were washed, fixated and stained with Crystal Violet (CV) followed by measurement of absorbance at 570 nm. Shown is % of CV staining relative to cells not treated with tunicamycin nor Sig-1R binder ±SEM of 3 independent experiments, *p<0.05.

FIG. 19 is a bar graph demonstrating the effect of Sig-1R binders on astrocytes survival under Tunicamycin-induced ER stress conditions. WT and Mut astrocytes were treated or not for 24 hours with 20, 30 or 50 μM of the indicated Sig-1R binders in the presence of 50 ng/ml Tunicamycin (Tun) during the last 22 hours. Following, the cells were washed, fixated and stained with Crystal Violet (CV) followed by measurement of absorbance at 570 nm. Shown is % of CV staining relative to cells not treated with tunicamycin nor Sig-1R binder ±SEM of 3 independent experiments, *p<0.05.

FIGS. 20A-B are bar graphs demonstrating that analogs of p8G5 (H8) improve mitochondrial health and cell survival following Tunicamycin-induced ER stress. FIG. 20A demonstrates the effect on mitochondrial membrane potential, as determined by JC1 fluorescent dye. Mut MEFs were incubated or not for 24 hours with 10 μM of the indicated compounds, washed and stained with Hoechst and JC1 followed by image-based single-cell analysis. FIG. 20B demonstrated the effect on cell survival under ER-stress conditions. Mut MEFs were incubated or not with the indicated concentrations of the indicated compounds for 2 hours followed by addition of 50 ng/ml Tunicamycin (Tun) for additional 22 hours, stained with Crystal Violet followed by measurement of absorbance at 570 nm. Shown is average normalized to Tun-treated cells without any compound ±SEM of 3 independent experiments. * not significant; ** p<0.02; *** all values p<0.04.

### DESCRIPTION OF SPECIFIC EMBODIMENTS OF THE INVENTION

The present invention, in some embodiments thereof, relates to methods of treating leukodystrophies.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Vanishing White Matter (VWM) disease is a recessive orphan disease characterized by progressive loss of white matter in both hemispheres of the brain, which is caused by mutations in any of the five genes encoding the eIF2B subunits.

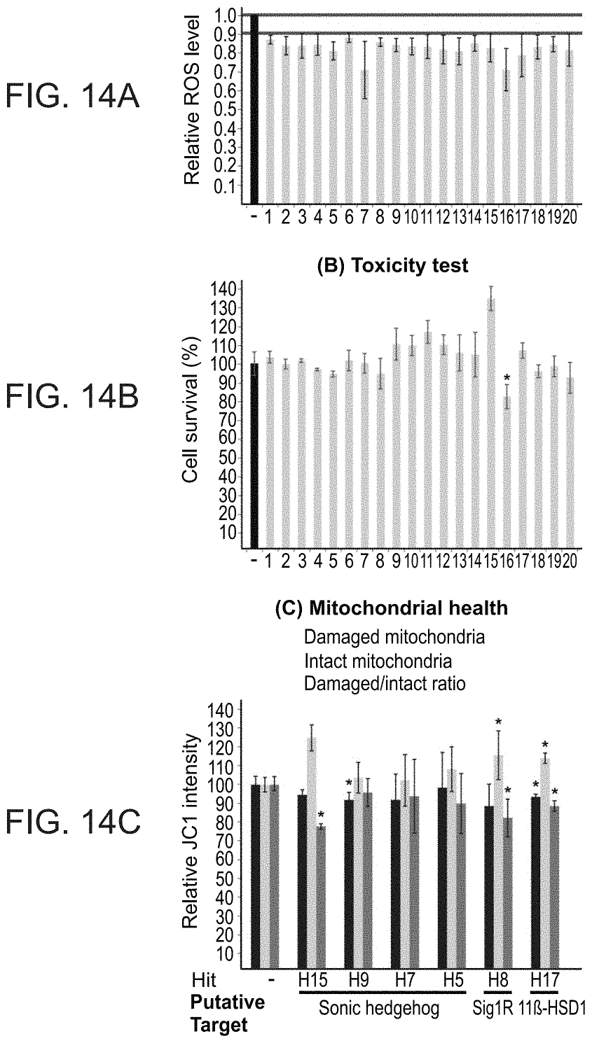

Using a novel screening method the present inventors have now uncovered several commercially available compounds that reduced the levels of reactive oxygen species in MEFs of eIF2B-mutant mice and enhanced mitochondrial health without significantly affecting cell survival (Example 1, FIGS. 14A-C and Tables 1-3). These results suggest the use of the identified compounds e.g. in the treatment of diseases associated with mitochondrial dysfunction, oxygen stress and/or ER stress, in general, and in leukodystrophies (e.g. VWM), in particular.

Furthermore, six of the identified compounds were mapped by the present inventors to bind three putative pathways: Sigma-1-Receptor (Sig-1R), Sonic hedgehog (SHH) and 11β-hydroxysteroid dehydrogenase type1 (11β-HSD1) (Example 1). Consequently, the present teachings suggest the use of these identified compounds e.g. for modulating expression and/or activity of Sig-1R, SHH and/or 11β-HSD1 in a cell and for treatment of diseases which can benefit from this modulation.

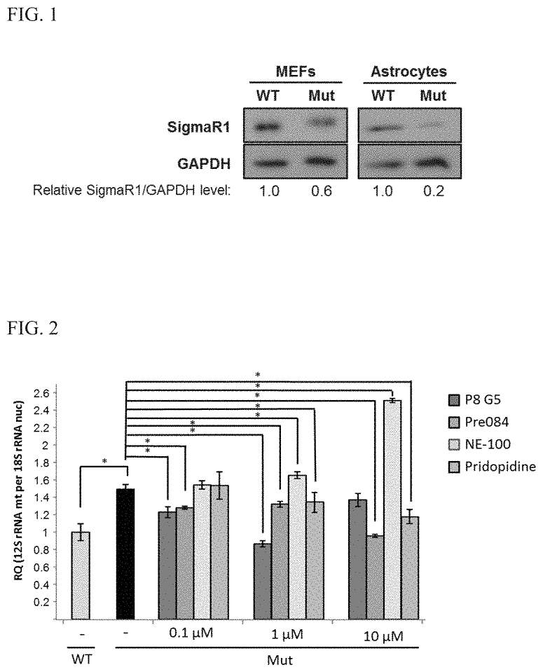

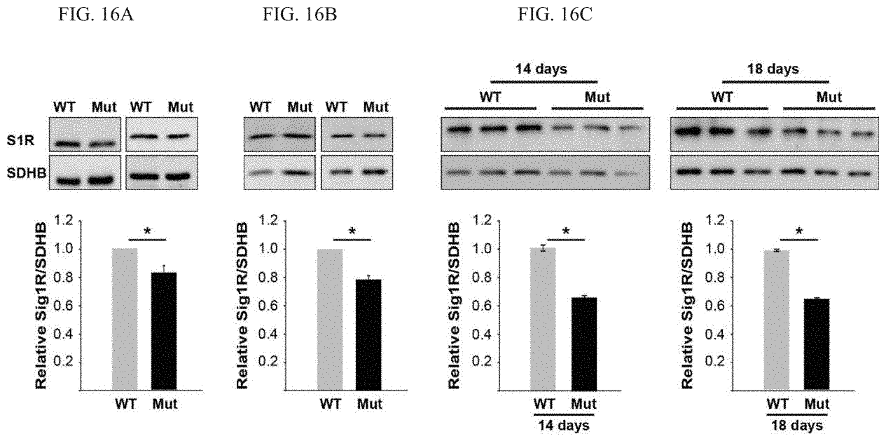

Following, the present inventors have uncovered that MEFs, primary astrocytes and postnatal brains of eIF2B-mutant mice express lower levels of Sigma-1 Receptor (Sig-1R) as compared to their wild-type counterparts (Example 2, FIGS. 1 and 16A-C. In addition, the present inventors have shown that several Sig-1R agonists[i.e. 4-[5-(3-methylphenoxy)pentyl]morpholine (denoted herein as p8G5) and structural analogs thereof, Pre084 and pridopidine) are able to increase mitochondrial health, mitochondrial membrane potential and effective oxidative respiration in eIF2B-mutant cells; and to increase their ability to cope with chronic ER stress (Example 2, FIGS. 2-10, 17A-C, 18-19 and 20A-B). These results suggest the use of an agent capable of modulating (e.g. up-regulating) activity and/or expression of Sig-1R or one of its targets e.g. in the treatment of leukodystophies, in general, and VWM disease, in particular.

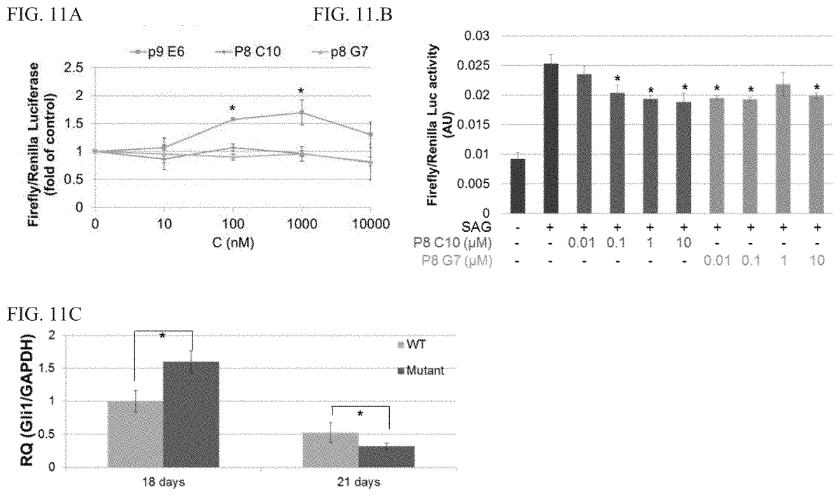

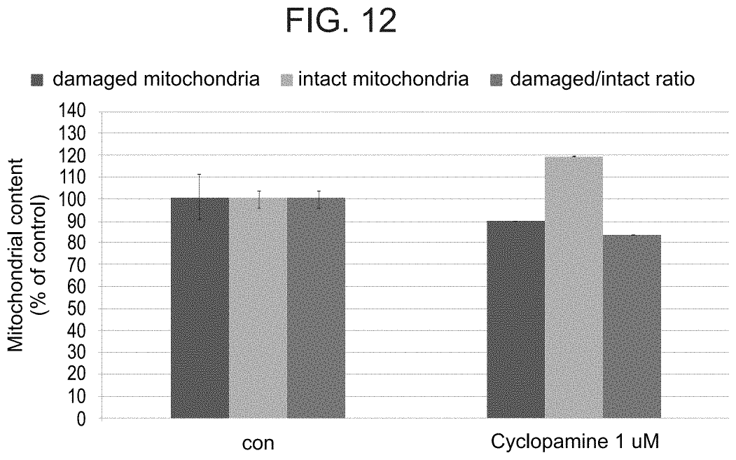

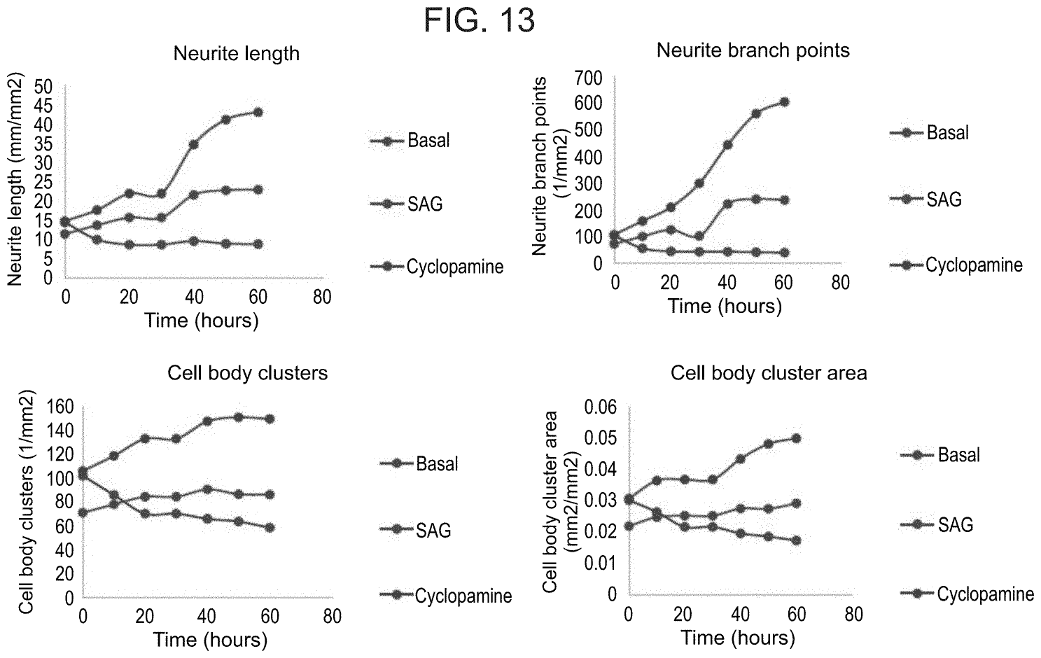

In addition, as noted above, the present inventors have discovered that several compounds [1-allyl-2-(3,4,5-trimethoxyphenyl)-1H-benzimidazole, 1-(2-fluorophenyl)-4-(phenylacetyl)piperazine) and 1-allyl-2-(2-phenylvinyl)-1H-benzimidazole]modulate activity of sonic hedgehog (SHH) signaling pathway (Example 3, FIGS. 11A-B). Following, the present inventors have uncovered that SHH pathway regulation is impaired in postnatal brains of eIF2B-mutant mice (Example 3, FIG. 11C). In addition, cyclopamine, a known inhibitor of SHH is able to increase mitochondrial health in eIF2B-mutant MEFs, and positively affect differentiation of oligodendrocytes precursor cells to mature oligodendrocytes (Example 3, FIGS. 12-13). Consequently, the present teachings suggest the use of these compounds e.g. in the treatment of diseases that can benefit from modulating the SHH signaling pathway.

Thus, according to an aspect of the present invention there is provided a method of treating a disease associated with mitochondrial dysfunction, oxidative stress and/or ER stress, the method comprising administering to the subject a therapeutically effective amount of a compound selected from the group consisting of 5-benzyl-2-[(2-chlorophenyl)imino]-1,3-thiazolidin-4-one, 5-butyl-3-{[2-(4-morpholinyl)ethyl]thio}-5H-[1,2,4]triazino[5,6-b]indole, 2-phenyl-N′-({5-[3-(trifluoromethyl)phenyl]-2-furyl}methylene)acetohydrazide, 1-{3-[(4-chlorobenzyl)oxy]phenyl}ethanone, 1-allyl-2-(3,4,5-trimethoxyphenyl)-1H-benzimidazole, 7-(difluoromethyl)-N-[2-(4-morpholinyl)ethyl]-5-phenylpyrazolo[1,5-a]pyrimidine-3-carboxamide, 1-phenyl-4-[4-(2-thienylcarbonyl)-1-piperazinyl]phthalazine, 4-[5-(3-methylphenoxy)pentyl]morpholine, 1-(2-fluorophenyl)-4-(phenylacetyl)piperazine, 2-{[2-oxo-2-(1-piperidinyl)ethyl]thio}-4-phenyl-6-(trifluoromethyl)pyrimidine, N-[2-(phenylthio)cyclohexyl]benzenesulfonamide, 1-{[3-(benzyloxy)phenyl]carbonothioyl}-4-methylpiperazine, 5-phenyl-N-(2-thienylmethyl)-7-(trifluoromethyl)pyrazolo[1,5-a]pyrimidine-2-carboxamide, 2-(2,3-dihydro-9H-imidazo[1,2-a]benzimidazol-9-yl)-1-(4-hydroxyphenyl)ethanone hydrobromide, 2-[(2,5-dimethoxyphenyl)diazenyl]-1-methyl-1H-benzimidazole, 2-[(2,6-dimethyl-1-piperidinyl)carbonyl]-7-methyl-5-phenylpyrazolo[ 1,5-a]pyrimidine, 2-(4-morpholinylmethyl)-1-(1-naphthylmethyl)-1H-benzimidazole, 5-isopropyl-N-methyl-3-phenylpyrazolo[1,5-a]pyrimidin-7-amine, 4-[5-(3,5-dimethylphenoxy)pentyl]morpholine, 4-[5-(3,4-dimethylphenoxy)pentyl]morpholine, 4-[6-(3-methylphenoxy)hexyl]morpholine, 4-[4-(3-methylphenoxy)butyl]morpholine, 4-[4-(3,4-dimethylphenoxy)butyl]morpholine, 4-[5-(3-methoxyphenoxy)pentyl]morpholine, 4-[5-(3-chlorophenoxy)pentyl]morpholine and 1-[5-(2-fluorophenoxy)pentyl]-4-methylpiperazine.

According to another aspect of the present invention there is provided a compound selected from the group consisting of from the group consisting of 5-benzyl-2-[(2-chlorophenyl)imino]-1,3-thiazolidin-4-one, 5-butyl-3-{[2-(4-morpholinyl)ethyl]thio}-5H-[ 1,2,4]triazino[5,6-b]indole, 2-phenyl-N′-({5-[3-(trifluoromethyl)phenyl]-2-furyl}methylene)acetohydrazide, 1-{3-[(4-chlorobenzyl)oxy]phenyl}ethanone, 1-allyl-2-(3,4,5-trimethoxyphenyl)-1H-benzimidazole, 7-(difluoromethyl)-N-[2-(4-morpholinyl)ethyl]-5-phenylpyrazolo[1,5-a]pyrimidine-3-carboxamide, 1-phenyl-4-[4-(2-thienylcarbonyl)-1-piperazinyl]phthalazine, 4-[5-(3-methylphenoxy)pentyl]morpholine, 1-(2-fluorophenyl)-4-(phenylacetyl)piperazine, 2-{[2-oxo-2-(1-piperidinyl)ethyl]thio}-4-phenyl-6-(trifluoromethyl)pyrimidine, N-[2-(phenylthio)cyclohexyl]benzenesulfonamide, 1-{[3-(benzyloxy)phenyl]carbonothioyl}-4-methylpiperazine, 5-phenyl-N-(2-thienylmethyl)-7-(trifluoromethyl)pyrazolo[1,5-a]pyrimidine-2-carboxamide, 2-(2,3-dihydro-9H-imidazo[ 1,2-a]benzimidazol-9-yl)-1-(4-hydroxyphenyl)ethanone hydrobromide, 2-[(2,5-dimethoxyphenyl)diazenyl]-1-methyl-1H-benzimidazole, 2-[(2,6-dimethyl-1-piperidinyl)carbonyl]-7-methyl-5-phenylpyrazolo[ 1,5-a]pyrimidine, 2-(4-morpholinylmethyl)-1-(1-naphthylmethyl)-1H-benzimidazole, 5-isopropyl-N-methyl-3-phenylpyrazolo[ 1,5-a]pyrimidin-7-amine, 4-[5-(3,5-dimethylphenoxy)pentyl]morpholine, 4-[5-(3,4-dimethylphenoxy)pentyl]morpholine, 4-[6-(3-methylphenoxy)hexyl]morpholine, 4-[4-(3-methylphenoxy)butyl]morpholine, 4-[4-(3,4-dimethylphenoxy)butyl]morpholine, 4-[5-(3-methoxyphenoxy)pentyl]morpholine, 4-[5-(3-chlorophenoxy)pentyl]morpholine and 1-[5-(2-fluorophenoxy)pentyl]-4-methylpiperazine for use in the treatment of a disease associated with mitochondrial dysfunction, oxidative stress and/or ER stress. According to specific embodiment, the compound is 1-allyl-2-(3,4,5-trimethoxyphenyl)-1H-benzimidazole, 1-(2-fluorophenyl)-4-(phenylacetyl)piperazine) and/or 1-allyl-2-(2-phenylvinyl)-1H-benzimidazole.

According to specific embodiment, the compound is 2-[(2,6-dimethyl-1-piperidinyl)carbonyl]-7-methyl-5-phenylpyrazolo[1,5-a]pyrimidine.

According to specific embodiments, the compound is 5-benzyl-2-[(2-chlorophenyl)imino]-1,3-thiazolidin-4-one, 5-butyl-3-{[2-(4-morpholinyl)ethyl]thio}-5H-[1,2,4]triazino[5,6-b]indole, 2-phenyl-N′-({5-[3-(trifluoromethyl)phenyl]-2-furyl}methylene)acetohydrazide, 1-{3-[(4-chlorobenzyl)oxy]phenyl}ethanone, 7-(difluoromethyl)-N-[2-(4-morpholinyl)ethyl]-5-phenylpyrazolo[1,5-a]pyrimidine-3-carboxamide, 2-{[2-oxo-2-(1-piperidinyl)ethyl]thio}-4-phenyl-6-(trifluoromethyl)pyrimidine, N-[2-(phenylthio)cyclohexyl]benzene sulfonamide, 1-{[3-(benzyloxy)phenyl]carbonothioyl}-4-methylpiperazine, 1-{[3-(benzyloxy)phenyl]carbonothioyl}-4-methylpiperazine, 5-phenyl-N-(2-thienylmethyl)-7-(trifluoromethyl)pyrazolo[ 1,5-a]pyrimidine-2-carboxamide, 2-(2,3-dihydro-9H-imidazo[ 1,2-a]benzimidazol-9-yl)-1-(4-hydroxyphenyl)ethanone hydrobromide, 2-(4-morpholinylmethyl)-1-(1-naphthylmethyl)-1H-benzimidazole, 2-(benzylthio)-N-cyclopentylbenzamide, and/or 5-isopropyl-N-methyl-3-phenylpyrazolo[1,5-a]pyrimidin-7-amine

The Examples section which follows demonstrate that 4-[5-(3-methylphenoxy)pentyl]morpholine and several structural analogs have a similar effect on mitochondrial health, oxidative respiration and ability to cope with chronic stress in the eIF2B-mutant cells.

Thus, according to another aspect of the present invention there is provided a method of treating a disease associated with mitochondrial dysfunction, oxidative stress and/or ER stress, the method comprising administering to the subject a therapeutically effective amount of a compound represented by Formula I:

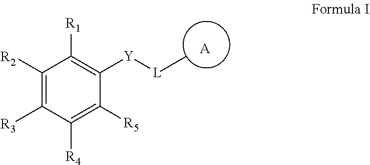

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;

Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety,

thereby treating the disease in the subject.

According to another aspect of the present invention there is provided a compound represented by Formula I:

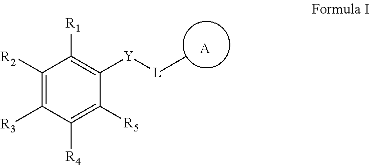

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;

Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety,

for use in the treatment of a disease associated with mitochondrial dysfunction, oxidative stress and/or ER stress.

In some of any of the embodiments described herein for compounds of Formula I, none of two or more of R₁-R₅ form together a heteroalicyclic moiety, as defined herein.

In some of any of the embodiments described herein for compounds of Formula I, none of two or more of R₁-R₅ form together a heterocyclic moiety (a heteroalicyclic or a heteroaryl, as defined herein).

In some of any of the embodiments described herein for compounds of Formula I, A is a heteroalicyclic or a heteroaryl heterocyclic moiety, as these terms are defined herein.

In some of any of the embodiments described herein for compounds of Formula I, A is a nitrogen-containing heterocyclic moiety.

By “nitrogen-containing heterocyclic moiety” are encompassed heteroalicyclic and heteroaryl moieties, as defined herein, containing one or more nitrogen atoms within the cyclic ring. Exemplary nitrogen-containing heterocyclic moieties include, but are not limited to, imidazole, morpholine, piperidine, piperazine, oxalidine, pyrrole, oxazole, thiazole, pyrazole, pyridine, pyrimidine, isoquinoline and purine.

The nitrogen-containing heterocylic moiety can be substituted or unsubstituted, and, when substituted, the moiety comprises one or more substituents as defined herein for heteroalicyclic and heteroaryl.

In some embodiments, A is a nitrogen-containing heteroalicyclic moiety.

In exemplary embodiments, A is morpholine.

In exemplary embodiments, A is piperazine.

In exemplary embodiments, A is N-methyl piperazine.

In some of any of the embodiments described herein, Y is O.

In some of any of the embodiments described herein, Y is O and A is a nitrogen-containing heteroalicyclic moiety.

In some of any of the embodiments described herein, L is a hydrocarbon chain.

Herein, the term “hydrocarbon” describes an organic moiety that includes, as its basic skeleton, a chain of carbon atoms, also referred to herein as a backbone chain, substituted mainly by hydrogen atoms. The hydrocarbon can be saturated or unsaturated, be comprised of aliphatic, alicyclic and/or aromatic moieties, and can optionally be substituted by one or more substituents (other than hydrogen).

A substituted hydrocarbon may have one or more substituents, whereby each substituent can independently be, for example, alkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine, and any other substituents as described herein (for example, as defined herein for R₁-R₅).

The hydrocarbon moiety can optionally be interrupted by one or more heteroatoms, including, without limitation, one or more oxygen (O), nitrogen (substituted or unsubstituted, as defined herein for —NR′—) and/or sulfur (S) atoms.

In some embodiments of any of the embodiments described herein relating to a hydrocarbon chain, the hydrocarbon is not interrupted by any heteroatom, nor does it comprise heteroatoms in its backbone chain, and can be an alkylene chain, or be comprised of alkyls, cycloalkyls, aryls, alkaryls, aralkyls, alkenes and/or alkynes, as defined herein, covalently attached to one another in any order.

In some embodiments, L in Formula I is or comprises an alkylene chain as defined herein.

In some embodiments, L in Formula I is an alkylene chain as defined herein.

The term “alkylene” describes a saturated aliphatic hydrocarbon group, as this term is defined herein. This term is also referred to herein as “alkyl” which is a linking moiety or group.

In some embodiments, when L is an alkylene chain, L can be represented by —(CR′R″)n-, wherein R′ and R″ are as defined herein, and each independently can be, for example, hydrogen, alkyl, cycloalkyl, aryl, heteroalicyclic, heteroaryl, alkoxy, aryloxy, thioalkoxy, thioaryloxy, hydroxyl, halogen, trihaloalkyl, trihaloalkoxy, amine, cyano, nitro, carbonyl, thiocarbonyl, carboxylate, thioacarboxylate, amide, thioamide, carbamate, thiocarbamate, alkaryl, aralkyl, sulfinyl, sylfonyl, sulfonate, and sulfonamide; and n is an integer of from 2 to 20, or from 2 to 10, or from 3 to 10, or from 4 to 10.

According to these embodiments, L is an alkylene chain that is composed of 2-20, or 2-10, or 3-10, or 4-10, or 4-8 or 4-6 (CR′R″) units.

R′ and R″ in each of these units can independently be the same or different.

In some of these embodiments, in all of the CR′R″ units in the alkylene chain, each of R′ and R″ is hydrogen. According to these embodiments, L is an unsubstituted alkylene chain.

When one or both of R′ and R″ in one of more of the CR′R″ units is other than hydrogen, L can represent a substituted alkylene chain.

In some of these embodiments, n is an integer ranging from 4 to 10, or from 4 to 8, or from 4 to 6.

In some of these embodiments, n is 4 or 5.

In some embodiments of any of the embodiments described herein relating to L as being or comprising a hydrocarbon chain, the hydrocarbon chain is interrupted by one or more heteroatoms.

Exemplary such hydrocarbons comprise one or more alkylene glycol groups or derivatives thereof.

As used herein, the term “alkylene glycol” describes a —[O—(CR′R″)z]y- group, with R′ and R″ being as defined herein (and/or as defined herein for R₁ and R₂), and with z being an integer of from 1 to 10, preferably, from 2 to 6, more preferably 2 or 3, and y being an integer of 1 or more. Preferably R′ and R″ are both hydrogen. When z is 2 and y is 1, this group is ethylene glycol. When z is 3 and y is 1, this group is propylene glycol. When y is greater than 1, this group is also referred to herein as “alkylene glycol chain”.

When y is greater than 4, the alkylene glycol chain is also referred to herein as poly(alkylene glycol) moiety. In some embodiments of the present invention, a poly(alkylene glycol) moiety can have from 2 to 10 alkylene glycol groups, such that y is, for example, 2 to 10, or from 2 to 8, or from 2 to 6, or from 3 to 4.

In some embodiments, the hydrocarbon chain is or comprises one or more alkylene glycol derivatives, in which one or more of the oxygen atoms is replaced by a sulfur atom and/or a —NR′— group, as defined herein, and/or one or more of R′ and R″ in one or more unit is other than hydrogen.

According to some of any of the embodiments described herein, L is or comprises one or more alkylene glycol groups, as defined herein.

The number of alkylene glycol groups can range from 1 to 20, or from 2 to 20, or from 2 to 10, or from 2 to 8, or from 2 to 6, or from 2 to 4, or from 2 to 3.

When 2 or more alkylene glycol units are present, the groups can be the same or different.

For example, R′ and R″ in each of these groups can independently be the same or different. Alternatively, or in addition, one or more alkylene glycol groups can differ from one another when one or both of the oxygen atoms is replaced by —NR′— or —S— in one or more units.

In some embodiments, in at least one, or in all of, the alkylene glycol units, R′ and R″ are each hydrogen.

In some of these embodiments, in all of the alkylene glycol units, each of R′ and R″ is hydrogen.

According to some of these embodiments, L is or comprises an unsubstituted alkylene glycol chain.

In some embodiments, one or both of R′ and R″ in one of more of the alkylene glycol groups is other than hydrogen, and L is or comprises a substituted alkylene glycol.

In some of any of the embodiments described herein for a hydrocarbon chain, the hydrocarbon chain has at least 4 atoms in length, for example, the hydrocarbon chain has from 4 to 20 atoms, or 4 to 10 atoms, or 4 to 8 atoms, or 4 to 6 atoms, in length.

According to some of any of the embodiments described herein, Y is O; A is a nitrogen-containing heteroalicyclic moiety; and L is an alkylene of 2-10, preferably of 4-10, or of 4-8, or of 4-6, carbon atoms in length. In some of these embodiments, the alkylene is unsubstituted.

According to some of any of the embodiments described herein, R₁-R₅ are each independently selected from hydrogen, alkyl, alkoxy, hydroxy, halo and thioalkoxy.

According to some of any of the embodiments described herein, R₁-R₅ are each independently selected from hydrogen, alkyl, alkoxy and halo.

According to some of any of the embodiments described herein, at least one of R₁-R₅ is other than hydrogen.

According to some of any of the embodiments described herein, at least one R₁-R₅ is an alkyl, preferably a lower alkyl, of 1-10, or 1-8, or 1-6, or 1-4, carbon atoms. In exemplary embodiments, the alkyl is methyl.

According to some of any of the embodiments described herein, R₁ and/or R₂ is an alkyl as defined herein.

According to some of any of the embodiments described herein, at least one of R₁-R₅ is halo.

According to some of any of the embodiments described herein, R₄ and/or R₅ is halo.

The halo can be, for example, fluoro and/or chloro.

According to some of any of the embodiments described herein, at least of R₁-R₅ is alkoxy, and is preferably a lower alkoxy which comprises a lower alkyl as defined herein. In exemplary embodiments, the alkoxy is methoxy.

According to some of any of the embodiments described herein, R₂ and/or R₄ is alkoxy.

As used herein, the term “amine” describes both a —NR′R″ group and a —NR′— group, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

The term “amine” is used herein to describe a —NR′R″ group in cases where the amine is an end group, as defined hereinunder, and is used herein to describe a —NR′— group in cases where the amine is a linking group.

Herein throughout, the phrase “end group” describes a group (a substituent) that is attached to another moiety in the compound via one atom thereof.

The phrase “linking group” describes a group (a substituent) that is attached to another moiety in the compound via two or more atoms thereof.

The term “alkyl” describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a lower size alkyl having 1 to 10 carbon atoms.

The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking group, as this phrase is defined hereinabove, which connects two or more moieties via at least two carbons in its chain.

The term “aminoalkyl” is used herein to describe an alkyl substituted by an amine, as defined herein. In some embodiments, the amine substitutes a terminal carbon atom in the alkyl.

The term “alkaryl” described an alkyl as defined herein, which is substituted by an aryl as defined herein.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking group, as this phrase is defined hereinabove, connecting two or more moieties at two or more positions thereof.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The aryl group can be an end group, as this term is defined hereinabove, wherein it is attached to a single adjacent atom, or a linking group, as this term is defined hereinabove, connecting two or more moieties at two or more positions thereof.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking group, as this phrase is defined hereinabove, connecting two or more moieties at two or more positions thereof. Representative examples are pyridine, pyrrole, oxazole, indole, purine and the like.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking group, as this phrase is defined hereinabove, connecting two or more moieties at two or more positions thereof. Representative examples are piperidine, piperazine, tetrahydrofurane, tetrahydropyrane, morpholino and the like.

The terms “hydroxyl” or “hydroxy”, as used herein, refer to an —OH group.

The term “alkenyl” describes an unsaturated alkyl, as defined herein, having at least two carbon atoms and at least one carbon-carbon double bond, e.g., allyl, vinyl, 3-butenyl, 2-butenyl, 2-hexenyl and i-propenyl. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “halo” or “halogen” refers to F, Cl, Br and I atoms as substituents. The term “alkoxy” refers to an —OR′ group, wherein R′ is alkyl or cycloalkyl, as defined herein.

The term “aryloxy” refers to an —OR′ group, wherein R′ is aryl, as defined herein.

The term “heteroaryloxy” refers to an —OR′ group, wherein R′ is heteroaryl, as defined herein.

The term “thioalkoxy” refers to an —SR′ group, wherein R′ is alkyl or cycloalkyl, as defined herein.

The term “thioaryloxy” refers to an —SR′ group, wherein R′ is aryl, as defined herein.

The term “thioheteroaryloxy” refers to an —SR′ group, wherein R′ is heteroaryl, as defined herein.

The term “hydroxyalkyl,” as used herein, refers to an alkyl group, as defined herein, substituted with one or more hydroxy group(s), e.g., hydroxymethyl, 2-hydroxyethyl and 4-hydroxypentyl.

The term “aminoalkyl,” as used herein, refers to an alkyl group, as defined herein, substituted with one or more amino group(s).

The term “alkoxyalkyl,” as used herein, refers to an alkyl group substituted with one or more alkoxy group(s), e.g., methoxymethyl, 2-methoxyethyl, 4-ethoxybutyl, n-propoxyethyl and t-butylethyl.

The term “trihaloalkyl” refers to —CX3, wherein X is halo, as defined herein. An exemplary haloalkyl is CF3.

A “guanidine” or “guanidine” or “guanidinyl” or “guanidyl” group refers to an —RaNC(═NRd)-NRbRc group, where each of Ra, Rb, Rc and Rd can each be as defined herein for R′ and R″.

A “guanyl” or “guanine” group refers to an RaRbNC(═NRd)-group, where Ra, Rb and Rd are each as defined herein for R′ and R″.

The term “cyano” describes a —C≡N group.

The term “nitro” describes an —NO₂ group.

The term “sulfate” describes a —O—S(═O)₂—OR′ end group, as this term is defined hereinabove, or an —O—S(═O)₂-O— linking group, as these phrases are defined hereinabove, where R′ is as defined hereinabove.

The term “thiosulfate” describes a —O—S(═S)(═O)—OR′ end group or a —O—S(=S)(═O)—O— linking group, as these phrases are defined hereinabove, where R′ is as defined hereinabove.

The term “sulfite” describes an —O—S(═O)—O—R′ end group or a —O—S(═O)—O— group linking group, as these phrases are defined hereinabove, where R′ is as defined hereinabove.

The term “thiosulfite” describes a —O—S(═S)—O—R′ end group or an —O—S(═S)—O— group linking group, as these phrases are defined hereinabove, where R′ is as defined hereinabove.

The term “sulfinate” describes a —S(═O)—OR′ end group or an —S(═O)—O— group linking group, as these phrases are defined hereinabove, where R′ is as defined hereinabove.

The term “sulfoxide” or “sulfinyl” describes a —S(═O)R′ end group or an —S(═O)— linking group, as these phrases are defined hereinabove, where R′ is as defined hereinabove.

The term “sulfonate” or “sulfonyl” describes a —S(═O)₂-R′ end group or an —S(═O)₂-linking group, as these phrases are defined hereinabove, where R′ is as defined herein.

The term “S-sulfonamide” describes a —S(═O)₂—NR′R″ end group or a —S(═O)₂—NR′— linking group, as these phrases are defined hereinabove, with R′ and R″ as defined herein. The term “N-sulfonamide” describes an R'S(═O)₂—NR″— end group or a —S(═O)₂—NR′— linking group, as these phrases are defined hereinabove, where R′ and R″ are as defined herein.

The term “carbonyl” or “carbonate” as used herein, describes a-C(═O)—R′ end group or a —C(═O)— linking group, as these phrases are defined hereinabove, with R′ as defined herein.

The term “thiocarbonyl” as used herein, describes a-C(═S)—R′ end group or a —C(═S)— linking group, as these phrases are defined hereinabove, with R′ as defined herein.

The term “oxo” as used herein, describes a (═O) group, wherein an oxygen atom is linked by a double bond to the atom (e.g., carbon atom) at the indicated position.

The term “thiooxo” as used herein, describes a (═S) group, wherein a sulfur atom is linked by a double bond to the atom (e.g., carbon atom) at the indicated position.

The term “oxime” describes a ═N—OH end group or a ═N—O— linking group, as these phrases are defined hereinabove.

The term “acyl halide” describes a —(C═O)R″″ group wherein R″″ is halo, as defined hereinabove.

The term “azo” or “diazo” describes an-N═NR′ end group or an-N═N— linking group, as these phrases are defined hereinabove, with R′ as defined hereinabove.

The term “azide” describes an-N₃ end group.

The term “carboxylate” as used herein encompasses C-carboxylate and O-carboxylate.

The term “C-carboxylate” describes a-C(═O)—OR′ end group or a —C(═O)—O— linking group, as these phrases are defined hereinabove, where R′ is as defined herein.

The term “O-carboxylate” describes a-OC(═O)R′ end group or a —OC(═O)— linking group, as these phrases are defined hereinabove, where R′ is as defined herein.

A carboxylate can be linear or cyclic. When cyclic, R′ and the carbon atom are linked together to form a ring, in C-carboxylate, and this group is also referred to as lactone. Alternatively, R′ and O are linked together to form a ring in O-carboxylate. Cyclic carboxylates can function as a linking group, for example, when an atom in the formed ring is linked to another group.

The term “thiocarboxylate” as used herein encompasses C-thiocarboxylate and 0-thiocarboxylate.

The term “C-thiocarboxylate” describes a-C(═S)—OR′ end group or a —C(═S)—O— linking group, as these phrases are defined hereinabove, where R′ is as defined herein.

The term “O-thiocarboxylate” describes a-OC(═S)R′ end group or a —OC(═S)— linking group, as these phrases are defined hereinabove, where R′ is as defined herein.

A thiocarboxylate can be linear or cyclic. When cyclic, R′ and the carbon atom are linked together to form a ring, in C-thiocarboxylate, and this group is also referred to as thiolactone. Alternatively, R′ and O are linked together to form a ring in O-thiocarboxylate. Cyclic thiocarboxylates can function as a linking group, for example, when an atom in the formed ring is linked to another group.

The term “carbamate” as used herein encompasses N-carbamate and O-carbamate.

The term “N-carbamate” describes an R″OC(═O)—NR′— end group or a —OC(═O)—NR′— linking group, as these phrases are defined hereinabove, with R′ and R″ as defined herein.

The term “O-carbamate” describes an-OC(═O)—NR′R″ end group or an —OC(═O)—NR′-linking group, as these phrases are defined hereinabove, with R′ and R″ as defined herein.

A carbamate can be linear or cyclic. When cyclic, R′ and the carbon atom are linked together to form a ring, in O-carbamate. Alternatively, R′ and O are linked together to form a ring in N-carbamate. Cyclic carbamates can function as a linking group, for example, when an atom in the formed ring is linked to another group.

The term “carbamate” as used herein encompasses N-carbamate and O-carbamate.

The term “thiocarbamate” as used herein encompasses N-thiocarbamate and O-thiocarbamate.

The term “O-thiocarbamate” describes a-OC(═S)—NR′R″ end group or a —OC(═S)—NR′-linking group, as these phrases are defined hereinabove, with R′ and R″ as defined herein.

The term “N-thiocarbamate” describes an R″OC(═S)NR′— end group or a —OC(═S)NR′-linking group, as these phrases are defined hereinabove, with R′ and R″ as defined herein.

Thiocarbamates can be linear or cyclic, as described herein for carbamates.

The term “dithiocarbamate” as used herein encompasses S-dithiocarbamate and N-dithiocarbamate.

The term “S-dithiocarbamate” describes a-SC(═S)—NR′R″ end group or a —SC(═S)NR′-linking group, as these phrases are defined hereinabove, with R′ and R″ as defined herein.

The term “N-dithiocarbamate” describes an R″SC(═S)NR′— end group or a —SC(═S)NR′-linking group, as these phrases are defined hereinabove, with R′ and R″ as defined herein.

The term “urea”, which is also referred to herein as “ureido”, describes a-NR′C(═O)-NR″R′″ end group or a —NR′C(═O)—NR″— linking group, as these phrases are defined hereinabove, where R′ and R″ are as defined herein and R′″ is as defined herein for R′ and R″.

The term “thiourea”, which is also referred to herein as “thioureido”, describes a —NR′-C(═S)—NR″R′″ end group or a-NR′—C(═S)—NR″— linking group, with R′, R″ and R′″ as defined herein.

The term “amide” as used herein encompasses C-amide and N-amide.

The term “C-amide” describes a-C(═O)—NR′R″ end group or a —C(═O)—NR′— linking group, as these phrases are defined hereinabove, where R′ and R″ are as defined herein.

The term “N-amide” describes a R′C(═O)—NR″— end group or a R′C(═O)—N— linking group, as these phrases are defined hereinabove, where R′ and R″ are as defined herein.

The term “hydrazine” describes a —NR′—NR″R′″ end group or a —NR′—NR″— linking group, as these phrases are defined hereinabove, with R′, R″, and R′″ as defined herein.

As used herein, the term “hydrazide” describes a-C(═O)—NR′—NR″R′″ end group or a —C(═O)—NR′—NR″— linking group, as these phrases are defined hereinabove, where R′, R″ and R′″ are as defined herein.

As used herein, the term “thiohydrazide” describes a-C(═S)—NR′—NR″R′″ end group or a —C(═S)—NR′—NR″— linking group, as these phrases are defined hereinabove, where R′, R″ and R′″ are as defined herein.

According to specific embodiment, the compound is 4-[5-(3-methylphenoxy)pentyl]morpholine, 4-[5-(3,5-dimethylphenoxy)pentyl]morpholine, 4-[5-(3,4-dimethylphenoxy)pentyl]morpholine, 4-[6-(3-methylphenoxy)hexyl]morpholine, 4-[4-(3-methylphenoxy)butyl]morpholine, 4-[4-(3,4-dimethylphenoxy)butyl]morpholine, 4-[5-(3-methoxyphenoxy)pentyl]morpholine, 4-[5-(3-chlorophenoxy)pentyl]morpholine and/or 1-[5-(2-fluorophenoxy)pentyl]-4-methylpiperazine, each possibility represents a separate embodiment of the present invention.

According to specific embodiment, the compound is 4-[5-(3-methylphenoxy)pentyl]morpholine.

The Examples section which follows further provides evidence that several Sig-1R agonists increased mitochondrial health, mitochondrial membrane potential and effective oxidative respiration in eIF2B-mutant cells; and increased their ability to cope with chronic ER stress; and thus support the use of Sig-1R pathway agonists for the treatment of leukodystrophies.

Thus, according to an aspect of the present invention there is provided a method of treating leukodystrophy in a subject in need thereof, the method comprising administering to the subject a therapeutically effective amount of an agent capable of modulating activity and/or expression of a component participating in a Sigma-1 Receptor (Sig-1R) signaling pathway, thereby treating the leukodystrophy in the subject.

According to another aspect of the present invention there is provided an agent capable of modulating activity and/or expression of a component participating in a Sig-1R signaling pathway, for use in the treatment of leukodystrophy.

According to another aspect of the present invention there is provided a method of treating leukodystrophy in a subject in need thereof, the method comprising administering to the subject a therapeutically effective amount of an agent capable of up-regulating activity and/or expression of a component participating in a Sigma-1 Receptor (Sig-1R) signaling pathway, thereby treating the leukodystrophy in the subject. According to another aspect of the present invention there is provided an agent capable of up-regulating activity and/or expression of a component participating in a Sig-1R signaling pathway, for use in the treatment of leukodystrophy.

The term “treating” refers to inhibiting, preventing or arresting the development of a pathology (disease, disorder or condition e.g. disease associated with mitochondrial dysfunction, oxidative stress and/or ER stress e.g. leukodystrophy e.g. VWM) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age and of any gender which suffer from the pathology. According to specific embodiments, this term encompasses individuals who are at risk to develop the pathology.

As used herein, the term “disease associated with mitochondrial dysfunction” means that cells with dysfunctional mitochondria drive onset and/or progression of the disease.

Methods for determining mitochondrial function are known in the art and are also disclosed in the Examples section which follows; and include, for example Respirometry, Measuring mitochondrial respiration using e.g. 02-dependent quenching of porphyrin-based phosphors, amperometric 02 sensors or phosphorescent probes, detection of oxidants using e.g. fluorescent-, chemiluminescent-, or electrochemical/nanoparticle-based approaches, measurement of membrane potential, ATP production via bioluminescence, Calcium retention capacity using e.g. Calcium Green-5N.

Non-limiting examples of diseases associated with mitochondrial dysfunction include, but are not limited to, cancer, cardiovascular and liver diseases, degenerative disorders, autoimmune diseases and disorder, aging, DNA mutations, oxidative stress disorders, various myopathies, HIV, AIDS, VWM (vanishing white matter disease), MRCD (mitochondrial respiratory chain disease), LHON (Leber's hereditary optic neuropathy); MELAS (mitochondrial myopathy, encephalomyopathy, lactic acidosis, stroke-like symptoms); Pearson syndrome; Leigh syndrome; NARP (neuropathy, ataxia, retinitis pigmentosa, and ptosis); MERRF (myoclonic epilepsy with ragged red fibers); KSS (Kearns-Sayre Syndrome); MNGIE (myo neurogenic gastrointestinal encephalopathy); Friedreich Ataxia; and Alpers' disease.

As used herein, the term “disease associated with oxygen stress” refers to a disease in which oxygen stress drive onset and/or progression of the disease.

The term “oxidative stress” refers to an imbalance between the systemic manifestation of reactive oxygen species and the ability to detoxify the reactive intermediates or to repair the resulting damage, manifested in the production of peroxides and free radicals. Oxidative stress can damage all components of the cell including proteins, lipids, RNA and DNA.

Methods of determining oxidative stress are known in the art and include, but are not limited to malondialdehyde (MDA), protein carbonyl (PCO), reduced glutathione (GSH) and its disulfide forms i.e. GSSG and GSSP levels, and the activities of glutathione S-transferase (GST) glutathione peroxidase (GPx), MDA, SOD, catalase, TBARS, NO, Liver peroxidation, LDH, AOPP and/or determining ROS levels as further described hereinbelow.

Non-limiting examples of diseases associated with oxidative stress include, but are not limited to, ADHD, cancer, mitochondrial disorder, neurodegenerative disorder, diseases of aging, impaired energy processing disorders, Parkinson's disease, Lafora disease, Alzheimer's disease, ALS, AIDS dementia, stroke, neuropathic pain, atherosclerosis, heart failure, myocardial infarction, stable angina, ischemic reperfusion injury, lung injury, cystic fibrosis, asthma, renal damage due to nephrotoxic agent, contrast nephropathy fragile X syndrome, sickle-cell disease, thalassemia, lichen planus, vitiligo, autism, Asperger syndrome, infection, chronic fatigue syndrome, depression, and radiation damage.

As used herein, the term “disease associated with ER stress” means that cells exhibiting ER stress drive onset and/or progression of the disease.

The term “ER stress” refers to an imbalance between the demand that a load of proteins makes on the ER and the actual folding capacity of the ER to meet that demand, manifested by accumulation of misfolded and unfolded proteins in the ER lumen.

Methods for determining ER stress are known in the art and disclosed for examples in Oslowski et al. Methods Enzymol. (2011; 490: 71-92, the contents of which are fully incorporated herein by reference; and include for examples, determining expression of ER stress response genes e.g. XBP1, CHOP, GRP78 (BIP), phosphorylated IRE1α, ATF6α; measuring XBP1 splicing; determining expression of apoptotic or pro-apoptotic genes e.g. Bax, Bcl-2, Caspase-3; detecting ER dilation by electron microscopy; and/or Real-time redox measurements.

Non-limiting examples of diseases associated with ER stress include but are not limited to cancer, an inflammatory disease, a metabolic disease (e.g. diabetes, the metabolic syndrome, obesity), infection, neurodegenerative disorder (e.g. Alzheimer's disease, Parkinson's disease, Huntington, amyotrophic lateral sclerosis, prion disease), Wolcott-Rallison syndrome, Wolfram Syndrome, ischemia/reperfusion injury, stroke, atherosclerosis, hypoxia and hypoglycemia.

According to specific embodiments, the disease associated with ER stress is a protein folding/misfolding disease such as, but not limited to, Alzheimer's disease, Parkinson's disease, amyotrophic lateral sclerosis (ALS), Creutzfeldt-Jakob disease, bovine spongiform encephalopathy (BSE), light chain amyloidosis (AL), Huntington's disease, spinobulbar muscular atrophy (Kennedy disease), Machado-Joseph disease, dentatorubral-pallidoluysian atrophy (Haw River Syndrome), spinocerebellar ataxia and the like.

According to specific embodiments the disease is an inflammatory disease.

Inflammatory Diseases—

Include, but are not limited to, chronic inflammatory diseases and acute inflammatory diseases.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like β-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Non-limiting examples of cancers can be any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcino sarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to specific embodiments, the cancer is selected from the group consisting of lung cancer, stomach cancer, esophagus cancer, pancreas cancer, prostate cancer, breast cancer, liver cancer, brain cancer, medulloblastoma, Basal cell carcinoma (BCC), cancer stem cells, rhabdomyosarcomas, glioma, multiple myeloma and chronic myelogenous leukemia (CML).

According to specific embodiments, the disease is selected from the group consisting of leukodystrophy, multiple sclerosis, cancer, OXPHOS diseases, lactic acidosis and stroke-like episodes (MELAS), myoclonus epilepsy with ragged red fibers (MERRF), deafness-dystonia syndrome (DDP), Parkinson disease, diabetes mellitus and sensorineural hearing impairment.

According to specific embodiment, the disease is leukodystrophy.

As used herein, the term “leukodystrophy” refers to a disease or disorder that is characterized by a progressive degeneration of the white matter of the brain. Typically degeneration of the white matter is due to disrupted growth, development or function of one or more glial cell types (astrocytes/oligodendrocytes/microglia) leading to disrupted growth, development or maintenance of the myelin sheath which insulates nerve cells.

According to specific embodiments, the leukodystrophy is a genetic disorder.

According to specific embodiments, the leukodystrophy is caused by a defect in at least one of the genes involved with the growth or maintenance of the myelin.

According to specific embodiments the term “leukodystrophy” does not include an autoimmune disease, wherein the subjects immune system attack the myelin or the myelin producing cells such as multiple sclerosis.

According to specific embodiments, the term “leukodystrophy” does not include multiple sclerosis.

Non limiting Examples of leukodystophies include vanishing white matter [VWM, also known as childhood ataxia with diffuse central nervous system hypomyelination (CACH)], Krabbe disease, adrenoleukodystrophy, adrenomyeloneuropathy, Aicardi-Goutieres syndrome, Alexanders disease, 18q deletion syndrome, Adult polyglucosan body disease (APBD), Aicardi-Goutières syndrome (AGS), AD adult-onset leukodystrophy (ADLD), Cerebroretinal microangiopathy w/calcifications & cysts (CRMCC), Cerebrotendinous xanthomatosis (CTX), Free sialic acid storage disorders, Fucosidosis, Hypomyelination w/atrophy of the basal ganglia & cerebellum (H-ABC), Hypomyelination and congenital cataract (HCC), L-2-hydroxyglutaric aciduria, Leukoencephalopathy w/brain stem & spinal cord involvement & lactate elevation (LBSL), Leukoencephalopathy w/thalamus and brain stem involvement & lactate elevation (LTBL), Megalencephalic leukodystrophy w/subcortical cysts (MLC), PSAP-related MLD, Multiple sulfatase deficiency (MSD), Hereditary diffuse leukoencephalopathy w/spheroids (HDLS) cerebral autosomal dominant arteriopathy with subcortical infarcts and leukoencephalopathy (CADASIL), Oculodentodigital dysplasia (ODDD), Canavan disease, cerebrotendinous xanthomatosis, metachromatic leukodystrophy, neonatal adrenoleukodystrophy, ovarioleukodystrophy syndrome, Pelizaeus-Merzbacher disease, Pelizaeus-Merzbacher-like disease 1 (PMLD1), Refsum disease, Van der Knaap syndrome, Zellweger syndrome, Pol III-related leukodystrophies, RNAse T2-deficient leukoencephalopathy, Sjögren-Larsson syndrome, SOX10-associated disorders, X-linked adrenoleukodystrophy (X-ALD) and Adrenomyeloneuropathy (AMN)—the adult onset of ALD, each possibility represents a separate embodiment of the present invention.

According to specific embodiments, the leukodystrophy is selected from the group consisting of vanishing white matter (VWM) disease, Krabbe disease, Metachromatic leukodystrophy, Pelizaeus-Merzbacher disease, Canavan disease, Adrenoleukodystrophy, Adrenomyeloneuropathy, Alexander disease, Cerebrotendineous xanthomatosis and Refsum disease. According to specific embodiments, the leukodystrophy is vanishing white matter (VWM) disease.

As used herein the term “vanishing white matter (VWM)” (OMIM #306896), also known as childhood ataxia with diffuse central nervous system hypomyelination (CACH), refers to a leukodystrophy caused by a mutation in any of the five genes encoding the eIF2B subunits and characterized by progressive loss of white matter in the brain.

According to specific embodiments, VWM is congenital VWM.

According to specific embodiments, VWM is classical VWM.

According to specific embodiments, VWM is adult form VWM.

The phrase “component participating in a Sigma-1 Receptor (Sig-1R) signaling pathway” encompasses Sig-1R, Sig-1R activators and Sig-1R effectors. Exemplary components are described for example in Su et al. (2016) Trends in Pharmacological Sciences, 37(4): 262-278, the contents of which are fully incorporated herein by reference; and include, but not limited to Sig-1R, CYC1, PHB, SLC25A11, SLC25A39, VSAC2, BiP, IRE1, RAC1, VDAC2, IP3R, Ankyrin, Insig, Emerin, RanBP2, ELMOD, UP1, C14orf1, CYP51A1, CFTR, EIF5A, GANAB, HSD17B1, 2HSPA5, NSDHL, RDH11, RPN2, SC4MOL, SEC61A2, SQLE, SURF4, TM7SF2, NACA2, PDZD11, RAF1, RPS27A, SEC61A2, TM7SF2, UBA52, UBC, XPO1, XPOT, CLN3, LBR, NUP205, RAE1. According to specific embodiments, the component participating in a Sigma-1 Receptor (Sig-1R) signaling pathway is selected from the group consisting of Sig-1R, CYC1, PHB, SLC25A11, SLC25A39, VSAC2, BiP, IRE1, RAC1, VDAC2, IP3R, Ankyrin and Insig.

According to specific embodiments, the component participating in a Sigma-1 Receptor (Sig-1R) signaling pathway is human.

According to specific embodiments, the component participating in a Sigma-1 Receptor (Sig-1R) signaling pathway is Sig-1R.

As used herein, the term “Sigma-1 Receptor (Sig-1R)”, also known as Sigma Non-Opioid Intracellular Receptor 1 and Aging-Associated Gene 8 Protein refers to the polynucleotide or polypeptide expression product of the SIGMAR1 gene (Gene ID: 10280). According to a specific embodiment, the Sig-1R refers to the human Sig-1R, such as provided in the following Accession Numbers: NM_001282205, NM_001282206, NM_001282207, NM_001282208, NM_001282209, NP_001269134, NP_001269135, NP_001269136, NP_001269137 and NP_001269138. According to a specific embodiment, the Sig-1R refers to the mouse Sig-1R, such as provided in the following Accession Numbers: NM_011014, NM_001286538, NM_001286539, NM_001286540, NM_001286541, NP_001273467, NP_001273468, NP_001273469, NP_001273470 and NP_001273480.

According to specific embodiments, the component is down-regulated in diseased cells as compared to control cells not afflicted with the disease. Methods of analyzing whether a particular component is down-regulated are known in the art, and may be effected on the RNA level (using techniques such as Northern blot analysis, RT-PCR and oligonucleotides microarray) and/or the protein level (using techniques such as ELISA, Western blot analysis, immunohistochemistry and the like, which may be effected using antibodies specific to the component).

As used herein the phrase “agent capable of up-regulating activity and/or expression of a component participating in a Sigma-1 Receptor (Sig-1R) signaling pathway” refers to an agent that induces and/or increases the biological function and/or expression (polynucleotide or polypeptide) of a component participating in a Sig-1R pathway.

As used herein, the term “up-regulating activity” refers to an increase of at least 5% in biological function and/or expression in the presence of the agent in comparison to same in the absence of the agent, as determined by e.g. PCR, ELISA, Western blot analysis, activity assay (e.g. enzymatic, kinase, binding), decreased no. of mitochondria (as determined by e.g. PCR), increased survival (as determined by e.g. MTT or crystal violet staining), decreased ROS levels (as determined by e.g. CellRox ROS detector), increased mitochondrial membrane potential (as determined by e.g. TMRE statining) and increased mitochondrial oxidative phosphorylation (as determined by e.g. a commercial kit such as Seahorse XF Cell Mito Stress test kit).

According to a specific embodiment, the increase is in at least 10%, 30%, 40% or even higher say, 50%, 60%, 70%, 80%, 90% or more than 100%.

According to specific embodiments, the biological function of the component is manifested in increased mitochondrial respiration and/or decreased ER stress.

According to a specific embodiment, the biological function is translocation of Sig-1R from the mitochondria-associated endoplasmic reticulum (ER) membrane (MAM), an interface between ER and mitochondrial; and interaction with its targets.

Upregulation of activity and/or expression of a component participating in a Sig-1R signaling pathway can be effected at the genomic level (i.e., activation of transcription via promoters, enhancers, regulatory elements), at the transcript level (i.e., correct splicing, polyadenylation, activation of translation) or at the protein level (i.e., post-translational modifications, interaction with substrates, antibodies, small molecules, peptides and the like).

Following is a list of agents capable of upregulating the expression level and/or activity of a component participating in a Sig-1R signaling pathway.

An agent capable of upregulating expression of a component participating in a Sig-1R signaling pathway may be an exogenous polynucleotide sequence designed and constructed to express at least a functional portion of the component. Accordingly, the exogenous polynucleotide sequence may be a DNA or RNA sequence encoding the component.

To express an exogenous component participating in a Sig-1R signaling pathway in mammalian cells, a polynucleotide sequence encoding the component is preferably ligated into a nucleic acid construct suitable for mammalian cell expression. Such a nucleic acid construct includes a promoter sequence for directing transcription of the polynucleotide sequence in the cell in a constitutive or inducible manner.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof. The construct may also include an enhancer element which can stimulate transcription up to 1,000 fold from linked homologous or heterologous promoters. The vector may or may not include a eukaryotic replicon. The construct may also include a Translation Initiator of Short 5′ UTR (TISU) element.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations.

The type of vector used by some embodiments of the invention will depend on the cell type transformed. The ability to select suitable vectors according to the cell type transformed is well within the capabilities of the ordinary skilled artisan and as such no general description of selection consideration is provided herein.

Recombinant viral vectors are useful for in vivo expression of the component participating in a Sig-1R signaling pathway since they offer advantages such as lateral infection and targeting specificity. Viral vectors can also be produced that are unable to spread laterally.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986]. Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. Other vectors can be used that are non-viral, such as cationic lipids, polylysine, and dendrimers.

It will be appreciated that upregulation of a component participating in a Sig-1R signaling pathway can be also effected by administration of cells expressing the component into the individual.

Administration of the cells expressing the component of some embodiments of the invention can be effected using any suitable route such as intravenous, intra peritoneal, intra kidney, intra gastrointestinal track, subcutaneous, transcutaneous, intramuscular, intracutaneous, intrathecal, epidural and rectal.

Cells expressing the component of some embodiments of the invention can be derived from either autologous or from allogeneic sources derived from non-autologous sources. According to specific embodiments, the cells can be derived from the individuals and transfected ex vivo with an expression vector containing the polynucleotide designed to express the component as described hereinabove. Since non-autologous cells are likely to induce an immune reaction when administered to the body several approaches have been developed to reduce the likelihood of rejection of non-autologous cells. These include either suppressing the recipient immune system or encapsulating the non-autologous cells or tissues in immunoisolating, semipermeable membranes before transplantation.

An agent capable of upregulating a component participating in a Sig-1R signaling pathway may also be any compound which is capable of increasing the transcription and/or translation of an endogenous DNA or mRNA encoding the component and thus increasing endogenous component activity.

Upregulation of a component participating in a Sig-1R signaling pathway can be also achieved at the protein level using e.g., antibodies, small molecules, peptides and the like. According to specific embodiments, the agent is a peptide. Thus, according to specific embodiments, the agent is an exogenous polypeptide including at least a functional portion of the component as further described herein.

According to specific embodiments, the agent is an antibody. The term “antibody” as used in this invention includes intact molecules as well as functional fragments thereof (such as Fab, F(ab′)₂, Fv, scFv, dsFv, or single domain molecules such as VH and VL) that are capable of binding to an epitope of an antigen.

The antibody may be a polyclonal or a monoclonal antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

According to specific embodiments, the antibody may be a human antibody or a humanized antibody.

Methods for humanizing non-human antibodies are well known in the art. And disclosed for example in Winter and co-workers [Jones et al., Nature, 321:522-525 (1986); Riechmann et al., Nature 332:323-327 (1988); Verhoeyen et al., Science, 239:1534-1536 (1988), U.S. Pat. No. 4,816,567].

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

It will be appreciated that targeting of particular compartment within the cell can be achieved using intracellular antibodies (also known as “intrabodies”). These are essentially SCA to which intracellular localization signals have been added (e.g., ER, mitochondrial, nuclear, cytoplasmic). This technology has been successfully applied in the art (for review, see Richardson and Marasco, 1995, TIBTECH vol. 13). Intrabodies have been shown to virtually eliminate the expression of otherwise abundant cell surface receptors and to inhibit a protein function within a cell (See, for example, Richardson et al., 1995, Proc. Natl. Acad. Sci. USA 92: 3137-3141; Deshane et al., 1994, Gene Ther. 1: 332-337; Marasco et al., 1998 Human Gene Ther 9: 1627-42; Shaheen et al., 1996 J. Virol. 70: 3392-400; Werge, T. M. et al., 1990, FEBS Letters 274:193-198; Carlson, J. R. 1993 Proc. Natl. Acad. Sci. USA 90:7427-7428; Biocca, S. et al., 1994, Bio/Technology 12: 396-399; Chen, S-Y. et al., 1994, Human Gene Therapy 5:595-601; Duan, L et al., 1994, Proc. Natl. Acad. Sci. USA 91:5075-5079; Chen, S-Y. et al., 1994, Proc. Natl. Acad. Sci. USA 91:5932-5936; Beerli, R. R. et al., 1994, J. Biol. Chem. 269:23931-23936; Mhashilkar, A. M. et al., 1995, EMBO J. 14:1542-1551; PCT Publication No. WO 94/02610 by Marasco et al.; and PCT Publication No. WO 95/03832 by Duan et al.).

According to specific embodiments, the agent is a small molecule.

According to specific embodiments, the agent induces activation of the biological function of the component.

According to other specific embodiments, the agent increases the biological function of the component.

According to specific embodiments, the agent binds directly the component.

According to other specific embodiments, the agent indirectly binds the component by acting through an intermediary molecule, for example the agent binds to or modulates a molecule that in turn binds to or activates the component.

The agent can be a naturally occurring activator or a functional derivative thereof; or non-naturally occurring activator.

According to specific embodiments, the agent is a full agonist, that is, the effect of the agent is equivalent to the effect of the naturally occurring activator (i.e. ligand).

According to other specific embodiments, the agent is a partial agonist, that is, the effect of the agent is lower than the maximal effect of the naturally occurring activator (i.e. ligand). The effect of the agent may be lower by at least 5%, at least 10%, at least 20%, at least 30%, at least 40% at least 50%, at least 60%, at least 70%, at least 80% or at least 90% as compared to the maximal effect of the naturally occurring activator.

According to yet other specific embodiments, the agent is a super agonist, that is, the effect of the agent is higher than the maximal effect of the naturally occurring activator (i.e. ligand). The effect of the agonist may be higher by at least 5%, at least 10%, at least 20%, at least 30%, at least 40% at least 50%, at least 60%, at least 70%, at least 80%, at least 90% or at least 2 fold, at least 4 fold, at least 5 fold or at least 10 fold as compared to the maximal effect of the naturally occurring activator.

According to specific embodiments, the agent upregulates activity of Sig-1R.

According to specific embodiments, the agent is a Sig-1R agonist.

According to specific embodiments, the agent detaches BiP from Sig-1R.

Non-limiting examples of agents that modulate (e.g. up-regulate) activity of Sig-1R include 4-[5-(3-methylphenoxy)pentyl]morpholine, 4-[5-(3,5-dimethylphenoxy)pentyl]morpholine, 4-[5-(3,4-dimethylphenoxy)pentyl]morpholine, 4-[6-(3-methylphenoxy)hexyl]morpholine, 4-[4-(3-methylphenoxy)butyl]morpholine, 4-[4-(3,4-dimethylphenoxy)butyl]morpholine, 4-[5-(3-methoxyphenoxy)pentyl]morpholine, 4-[5-(3-chlorophenoxy)pentyl]morpholine and 1-[5-(2-fluorophenoxy)pentyl]-4-methylpiperazine, Pre-084, pridopidine, dextromethorphan, SA4503, Pentazosine, SKF-10047, 3-ppp, Fluvoxamine, Igmesine, Pregnenolone-S, DHEA-S, Donepezil, PPBP, Clorgyline, Fluoxetine, Imipramine, Sertaline, Carbetapentane, Dimemorfan, Amantadine, Memantine, Cocaine, BD 737, 4-IBP, OPC-14523, Anavex 2-73, Amitriptyline, L-687,384, Dimethyltryptamine, Methylphenylpiracetam and SOMCL-668.

According to specific embodiments, the agent is a compound represented by Formula I:

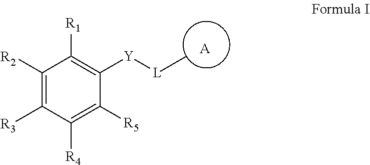

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;

Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety.

According to specific embodiments, the agent is 4-[5-(3-methylphenoxy)pentyl]morpholine, 4-[5-(3,5-dimethylphenoxy)pentyl]morpholine, 4-[5-(3,4-dimethylphenoxy)pentyl]morpholine, 4-[6-(3-methylphenoxy)hexyl]morpholine, 4-[4-(3-methylphenoxy)butyl]morpholine, 4-[4-(3,4-dimethylphenoxy)butyl]morpholine, 4-[5-(3-methoxyphenoxy)pentyl]morpholine, 4-[5(3-chlorophenoxy)pentyl]morpholine and/or 1-[5-(2-fluorophenoxy)pentyl]-4-methylpiperazine, each possibility represents a separate embodiment of the present invention.

According to specific embodiments, the agent is 4-[5-(3-methylphenoxy)pentyl]morpholine, Pre-084, pridopidine, dextromethorphan, SA4503, pentazocine, SKF-10047, 3-ppp, Fluvoxamine, Igmesine, Pregnenolone-S, DHEA-S, Donepezil, PPBP, Clorgyline, Fluoxetine, Imipramine, Sertaline, Carbetapentane, Dimemorfan, Amantadine, Memantine, Cocaine, BD 737, 4-IBP, OPC-14523, Anavex 2-73, Amitriptyline, L-687,384, Dimethyltryptamine, Methylphenylpiracetam and/or SOMCL-668, each possibility represents a separate embodiment of the present invention.

According to specific embodiments, the agent is 4-[5-(3-methylphenoxy)pentyl]morpholine.

“4-[5-(3-methylphenoxy)pentyl]morpholine”, PubChem CID 2202905, can be obtained from e.g. ChemBridge (ChemBridge ID #5320691).

“4-[5-(3,5-dimethylphenoxy)pentyl]morpholine”, “4-[5-(3,4-dimethylphenoxy)pentyl]morpholine” 4-[6-(3-methylphenoxy)hexyl]morpholine”, “4-[4-(3-methylphenoxy)butyl]morpholine”, “4-[4-(3,4-dimethylphenoxy)butyl]morpholine”, “4-[5-(3-methoxyphenoxy)pentyl]morpholine”, “4-[5-(3-chlorophenoxy)pentyl]morpholine” and “1-[5-(2-fluorophenoxy)pentyl]-4-methylpiperazine, can be obtained from e.g. ChemBridge (ChemBridge ID #5476348, 5359767, 5316063, 5319949, 5365072, 5364299, 5358807, 6161679).

According to specific embodiments, the agent is Pre-084. “Pre-084”, refers to 2-morpholin-4-ylethyl 1-phenylcyclohexane-1-carboxylate, CAS NO. 138847-85-5, can be obtained from e.g. Sigma-Aldrich.

According to specific embodiments, the agent is pridopidine.

“Pridopidine” (also known as ACR16, TV-7820, Huntexil) refers to 4-(3-(Methylsulfonyl)phenyl)-1-propylpiperidine (Chemical Registry number 882737-42-0, US Patent Application Publication No. US-2013-0267552), can be obtained from Teva Pharmaceuticals International. According to specific embodiments, pridopidine is pridopidine analog such as disclosed for example in US Patent Application Publication no. US 20150374677, the contents of which are fully incorporated herein by reference.

According to specific embodiments, the agent is Anavex 2-73.

“Anavex 2-73” (also known as 195615-84-0; AVex-73 hydrochloride; Anavex2-73; AE-hydrochloride) refers to 1-(2,2-diphenyloxolan-3-yl)-N,N-dimethylmethanamine; hydrochloride (PubChem CID 46932299, can be obtained from e.g. MedChemExpress.

Each of the compounds described herein can be utilized in its free base form or as a pharmaceutically acceptable salt.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound.

A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt can be an acid addition salt comprising at least one basic (e.g., amine) group of the compound which is in a positively charged form (e.g., an ammonium ion), in combination with at least one counter-ion, derived from the selected acid, that forms a pharmaceutically acceptable salt. The acid addition salts of the compounds described herein may therefore be complexes formed between one or more amino groups of the compound and one or more equivalents of an acid.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt, trifluoroacetic acid which affords a trifluoroacetic acid addition salt and lactic acid which affords a lactic acid addition salt.

Depending on the stoichiometric proportions between the charged group(s) in the compound and the counter-ion in the salt, the acid additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

According to specific embodiments, pridopidine is pridopidine L-tartrate e.g. pridopidine mono L-tartrate, pridopidine besylate Form Bl., pridopidine fumarate Form Al, pridopidine fumarate Form B 1, pridopidine fumarate Form CI, pridopidine gentistate, pridopidine glycolate, pridopidine L-malate, pridopidine napthalene 2-sulfonate, pridopidine oxalate, pridopidine succinate, pridopidine succinate or pridopidine tosylate (described e.g. in International Patent Application Publication no. WO2016106142, the contents of which are fully incorporated herein by reference).

The compounds described herein may possess asymmetric carbon atoms (optical centers) or double bonds; the racemates, diastereomers, geometric isomers and individual isomers are encompassed within the scope of the present invention.

As used herein, the term “enantiomer” describes a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. Typically, enantiomers diffract polarized light in opposite directions. In the context of the present embodiments, a compound may exhibit one or more chiral centers, each of which exhibiting an R- or an S-configuration and any combination, and compounds according to some embodiments of the present invention, can have any their chiral centers exhibit an R- or an 5-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound of the present invention, having one or more carboxylic acid moieties, which is administered as an ester (the “prodrug”). Such a prodrug is hydrolyzed in vivo, to thereby provide the free compound (the parent drug). The selected ester may affect both the solubility characteristics and the hydrolysis rate of the prodrug.

The compounds described herein can exist in unsolvated forms as well as solvated forms, including hydrated forms. In general, the solvated forms are equivalent to unsolvated forms and are encompassed within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

Certain compounds of the present invention may exist in multiple crystalline or amorphous forms. In general, all physical forms are equivalent for the uses contemplated by the present embodiments and are intended to be within the scope of the present invention.

Processes of synthesis of pridopidine and a pharmaceutically acceptable salt thereof are disclosed e.g. in U.S. Pat. No. 7,923,459, the contents of which are fully incorporated herein by reference.

The present inventors now discovered several agents which modulate activity of Sig-1R, SHH and 11β-HSD1.

Specifically, the present inventors now discovered that 4-[5-(3-methylphenoxy)pentyl]morpholine up-regulates activity of Sig-1R; that 1-allyl-2-(2-phenylvinyl)-1H-benzimidazole up-regulates activity of SHH; that 1-allyl-2-(3,4,5-trimethoxyphenyl)-1H-benzimidazole and 1-(2-fluorophenyl)-4-(phenylacetyl)piperazine) down-regulates activity of SHH; and that 2-[(2,6-dimethyl-1-piperidinyl)carbonyl]-7-methyl-5-phenylpyrazolo[1,5-a]pyrimidine binds 11β-HSD1.

Thus, according to an aspect of the present application, there is provided a method of modulating activity of Sig-1R in a cell, the method comprising contacting the cell with 4-[5-(3-methylphenoxy)pentyl]morpholine, thereby modulating activity of the Sig-1R.

Thus, according to an aspect of the present application, there is provided a method of up-regulating activity of Sig-1R in a cell, the method comprising contacting the cell with 4-[5-(3-methylphenoxy)pentyl]morpholine, thereby activating the Sig-1R.

According to another aspect of the present application, there is provided a method of modulating activity of Sigma-1 Receptor (Sig-1R) in a cell, the method comprising contacting the cell with a compound represented by Formula I:

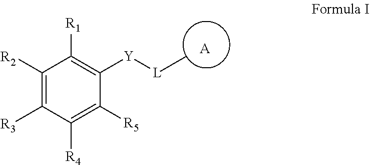

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;

Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety, thereby modulating activity of the Sig-1R.

According to another aspect of the present application, there is provided a method of up-regulating activity of Sigma-1 Receptor (Sig-1R) in a cell, the method comprising contacting the cell with a compound represented by Formula I:

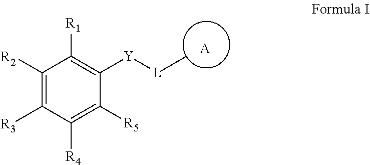

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;

Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety, thereby activating the Sig-1R.

According to specific embodiments, the method comprising analyzing activity of Sig-1R signaling pathway.

Thus, according to an aspect of the present application, there is provided a method of up-regulating activity of Sigma-1 Receptor (Sig-1R) in a cell, the method comprising:

(a) contacting the cell with 4-[5-(3-methylphenoxy)pentyl]morpholine, thereby activating the Sig-1R; and

(b) analyzing activity of Sig-1R signaling pathway.

According to another aspect of the present application, there is provided a method of up-regulating activity of Sigma-1 Receptor (Sig-1R) in a cell, the method comprising:

(a) contacting the cell with a compound represented by Formula I:

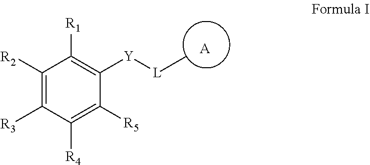

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;

Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety, thereby activating the Sig-1R; and

(b) analyzing activity of Sig-1R signaling pathway.

The cells may be of human or non-human origin.

According to specific embodiments, the cell is a human cell.

Non-limiting examples of cells types that can be used with specific embodiments of the present invention include, but are not limited to, neuronal cells, fibroblasts, astrocytes, olygodendrocytes, epithelial cells, endothelial cells, keratinocytes, myoblasts, cardiomyocytes, chondrocytes, osteoblasts, osteoclasts, hepatocytes, pancreatic cells (e.g. beta-cells), bone marrow cells, lymphocytes, macrophages, neutrophils, fibroblasts.

According to specific embodiments, the cells are fibroblasts.

According to specific embodiments, the cells are astrocytes.

The cells may be a primary cells or cells from an established cell line.

The cells may be immortalized cells, transformed cells

The cells may be stem cells (e.g. adult or embryonic stem cells, hematopoietic stem cells), somatic cells, germ cells, progenitor cells or terminally differentiated cells.

According to specific embodiments, the cells are comprised in a biological sample (e.g. body fluids such as whole blood, serum, plasma, cerebrospinal fluid, urine, lymph fluids, and various external secretions of the respiratory, intestinal and genitourinary tracts, tears, saliva, milk as well as white blood cells, tissue biopsy, amniotic fluid, chorionic villi)

According to specific embodiments, the cells are comprised in a tissue (e.g. soft tissue, hard tissue).

According to specific embodiments, the cells are comprised in a subject.

According to specific embodiments, the cells are diseased cells.

The cells can be freshly isolated or following storage e.g., at 4° C. or cryopreserved (i.e. frozen) at e.g. liquid nitrogen.

According to specific embodiments, the contacting is effected in-vivo.

According to specific embodiments, the contacting is effected in-vitro or ex-vivo.

Analyzing activity of Sig-1R may be effected by any method known in the art, including but not limited to kinase assays, determining translocation of Sig-1R from the mitochondria-associated endoplasmic reticulum (ER) membrane (MAM), and/or determining interaction with its targets (e.g. binding assays).

According to another aspect of the present application, there is provided a method of treating a disease that can benefit from modulating activity of Sigma-1 Receptor (Sig-1R), the method comprising administering to the subject a therapeutically effective amount of a compound represented by Formula I:

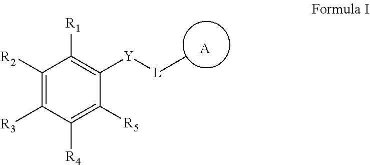

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide; Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety,

thereby treating the disease in the subject.

According to another aspect of the present application, there is provided a method of treating a disease that can benefit from up-regulating activity of Sigma-1 Receptor (Sig-1R), the method comprising administering to the subject a therapeutically effective amount of a compound represented by Formula I:

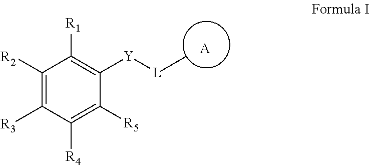

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;

Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety, thereby treating the disease in the subject.

According to another aspect of the present application, there is provided a compound represented by Formula I:

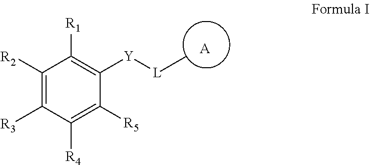

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;

Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety,

for use in the treatment of a disease that can benefit from modulating activity of Sigma-1 Receptor (Sig-1R).

According to another aspect of the present application, there is provided a compound represented by Formula I:

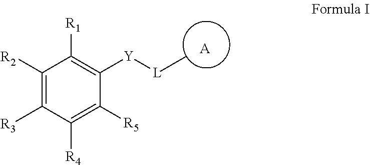

wherein:

R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide; Y is selected from O, S and NR′, wherein R′ is selected from hydrogen, alkyl, cycloalkyl, alkaryl, cycloalkyl and aryl;

L is a substituted or unsubstituted, saturated or unsaturated hydrocarbon chain of 2 to 10 carbon atoms in length, optionally interrupted by one or more heteroatoms selected from O, S and NR′; and

A is a heterocyclic moiety,

for use in the treatment of a disease that can benefit from up-regulating activity of Sigma-1 Receptor (Sig-1R).

According to another aspect of the present application, there is provided a method of treating a disease that can benefit from modulating activity of Sig-1R, the method comprising administering to the subject a therapeutically effective amount of 4-[5-(3-methylphenoxy)pentyl]morpholine, thereby treating the disease in the subject.

According to another aspect of the present application, there is provided a 4-[5-(3-methylphenoxy)pentyl]morpholine for use in the treatment of a disease that can benefit from modulating activity of Sigma-1 Receptor (Sig-1R).

According to another aspect of the present application, there is provided a method of treating a disease that can benefit from up-regulating activity of Sig-1R, the method comprising administering to the subject a therapeutically effective amount of 4-[5-(3-methylphenoxy)pentyl]morpholine, thereby treating the disease in the subject.

According to another aspect of the present application, there is provided a 4-[5-(3-methylphenoxy)pentyl]morpholine for use in the treatment of a disease that can benefit from up-regulating activity of Sigma-1 Receptor (Sig-1R).

Disease that can benefit from up-regulating activity of Sig-1R known in the art and disclosed e.g. in WO2013086425, WO2015112601, WO2016138135, the contents of which are fully incorporated herein by reference.

Non-limiting Examples of such diseases include, but are not limited to neurodegenerative diseases, Huntington's Disease, Parkinson's disease, iatrogenic and non-iatrogenic Parkinsonism, dyskinesias, dystonias, Tourette's disease, iatrogenic and non-iatrogenic psychoses and hallucinoses, schizophrenia disorder or schizophreniform disorder, mood and anxiety disorders, sleeping disorders, manic depressive illness, depression, obsessive-compulsive disease, a sleep disorder, autism spectrum disorder, ADHD, age-related cognitive impairment, abuse of alcohol and substances used as narcotics, cognitive disease, Alzheimer's disease, Creutzfeldt-Jakob disease, dead trauma, Huntington's disease, HIV disease, Pick's disease, diffuse Lewy body dementia, Rett syndrome, pain in conditions characterized by increased muscular tone, movement disorders and Movement disorders induced by drugs.

According to specific embodiments, the disease is associated with mitochondrial dysfunction, oxidative stress and/or ER stress. According to specific embodiments, the diseased cells express a decreased amount of Sig-1R as compared to control cells not afflicted with the disease.

According to another aspect of the present application, there is provided a method of modulating activity of sonic hedgehog (SHH) signaling pathway in a cell, the method comprising contacting the cell with a compound selected from the group consisting of 1-allyl-2-(3,4,5-trimethoxyphenyl)-1H-benzimidazole, 1-(2-fluorophenyl)-4-(phenylacetyl)piperazine) and 1-allyl-2-(2-phenylvinyl)-1H-benzimidazole.

According to specific embodiments, the method comprising analyzing activity of SHH signaling pathway.

Thus, according to another aspect of the present invention there is provided a method of modulating activity of sonic hedgehog (SHH) signaling pathway in a cell, the method comprising:

(a) contacting the cell with a compound selected from the group consisting of 1-allyl-2-(3,4,5-trimethoxyphenyl)-1H-benzimidazole, 1-(2-fluorophenyl)-4-(phenylacetyl)piperazine) and 1-allyl-2-(2-phenylvinyl)-1H-benzimidazole; and

(b) analyzing activity of SHH signaling pathway.

“1-allyl-2-(3,4,5-trimethoxyphenyl)-1H-benzimidazole” can be obtained from e.g. ChemBridge (ChemBridge ID #7669865).

“1-(2-fluorophenyl)-4-(phenylacetyl)piperazine)” can be obtained from e.g. ChemBridge (ChemBridge ID #6766109).

“1-allyl-2-(2-phenylvinyl)-1H-benzimidazole” can be obtained from e.g. ChemBridge (ChemBridge ID #5959162).

According to another aspect of the present application, there is provided a method of treating a disease that can benefit from modulating activity of sonic hedgehog (SHH) signaling pathway, the method comprising administering to the subject a therapeutically effective amount of a compound selected from the group consisting of 1-allyl-2-(3,4,5-trimethoxyphenyl)-1H-benzimidazole, 1-(2-fluorophenyl)-4-(phenylacetyl)piperazine and 1-allyl-2-(2-phenylvinyl)-1H-benzimidazole, thereby treating the disease in the subject.

According to another aspect of the present application, there is provided a compound selected from the group consisting of 1-allyl-2-(3,4,5-trimethoxyphenyl)-1H-benzimidazole, 1-(2-fluorophenyl)-4-(phenylacetyl)piperazine and 1-allyl-2-(2-phenylvinyl)-1H-benzimidazole, for use in the treatment of a disease that can benefit from modulating activity of sonic hedgehog (SHH) signaling pathway.

The phrase “component participating in a sonic hedgehog (SHH) signaling pathway” encompasses SHH, SHH activators and SHH effectors. Non-limiting exemplary components participating in a SHH signaling pathways are described e.g. in Ingham and McMahon (2001) Genes Dev. 15(23):3059-87, the contents of which are fully incorporated herein by reference, and include SHH, smoothened, Ptch1, and Gli.

According to specific embodiments, the component participating in a SHH signaling pathway is a human component.

According to specific embodiments, the component is down-regulated in diseased cells as compared to control cells not afflicted with the disease.

According to other specific embodiments, the component is up-regulated in diseased cells as compared to control cells not afflicted with the disease.

Methods of analyzing whether a particular component is down-regulated or up-regulated are known in the art, and may be effected on the RNA level (using techniques such as Northern blot analysis, RT-PCR and oligonucleotides microarray) and/or the protein level (using techniques such as ELISA, Western blot analysis, immunohistochemistry and the like, which may be effected using antibodies specific to the component).

According to specific embodiments, the component participating in a SHH signaling pathway is SHH.

As used herein, the term “sonic hedgehog (SHH)”, refers to the polynucleotide or polypeptide expression product of the SHH gene (Gene ID: 6469). According to a specific embodiment, the SHH refers to the human SHH, such as provided in the following Accession Numbers: NM_000193, NM_001310462, NP_000184 and NP_001297391. According to a specific embodiment, the SHH refers to the mouse SHH, such as provided in the following Accession Numbers: NM_009170 and NP_033196.

As used herein, the term “modulating” refers to altering activity and/or expression either by up-regulating or by down-regulating.

As used herein, the term “modulating activity and/or expression” refers to a change of at least 5% in biological function and/or expression in the presence of the agent in comparison to same in the absence of the agent, as determined by e.g. PCR, ELISA, Western blot analysis, activity assays (e.g. enzymatic activity assay, kinase activity assay, binding assay and the like). According to a specific embodiment, the change is in at least 10%, 30%, 40% or even higher say, 50%, 60%, 70%, 80%, 90% or more than 100%.

Analyzing activity of SHH signaling pathway may be effected by any method known in the art, including but not limited to determining expression of Gli1 transcription factor as further disclosed in the Examples section which follows.

According to specific embodiments, modulating activity and/or expression is down-regulating activity and/or expression.

Thus, according to specific embodiments, wherein said modulating is down-regulating, the compound is selected from the group consisting of 1-allyl-2-(3,4,5-trimethoxyphenyl)-1H-benzimidazole and 1-(2-fluorophenyl)-4-(phenylacetyl)piperazine.

Hence, according to another aspect of the present application, there is provided a method of treating a disease that can benefit from down-regulating activity of sonic hedgehog (SHH) signaling pathway, the method comprising administering to the subject a therapeutically effective amount of a compound selected from the group consisting of 1-allyl-2-(3,4,5-trimethoxyphenyl)-1H-benzimidazole and 1-(2-fluorophenyl)-4-(phenylacetyl)piperazine, thereby treating the disease in the subject.

According to another aspect of the present application, there is provided a compound selected from the group consisting of 1-allyl-2-(3,4,5-trimethoxyphenyl)-1H-benzimidazole and 1-(2-fluorophenyl)-4-(phenylacetyl)piperazine for use in the treatment of a disease that can benefit from down-regulating activity of sonic hedgehog (SHH) signaling pathway.

Disease that can benefit from down-regulating activity of SHH are known in the art and include but are not limited to cancer and multiple sclerosis.

According to specific embodiments, the disease is associated with mitochondrial dysfunction, oxidative stress and/or ER stress.

According to specific embodiments, modulating activity and/or expression is up-regulating activity and/or expression.

Thus, according to specific embodiments, wherein modulating activity is up-regulating activity, the compound is 1-allyl-2-(2-phenylvinyl)-1H-benzimidazole.

Hence, according to another aspect of the present application, there is provided a method of treating a disease that can benefit from up-regulating activity of sonic hedgehog (SHH) signaling pathway, the method comprising administering to the subject a therapeutically effective amount of 1-allyl-2-(2-phenylvinyl)-1H-benzimidazole, thereby treating the disease in the subject.

According to another aspect of the present application, there is provided a 1-allyl-2-(2-phenylvinyl)-1H-benzimidazole for use in the treatment of a disease that can benefit from up-regulating activity of sonic hedgehog (SHH) signaling pathway.

Disease that can benefit from up-regulating activity of SHH are known in the art and include but are not limited to muscular dystrophy (e.g., Duchenne Muscular Dystrophy), skeletal muscle regeneration following injury and/or brain recovery following ischemic stroke, hair loss.

According to specific embodiments, the disease is skeletal muscle regeneration following injury and/or brain recovery following ischemic stroke.

According to another aspect of the present application, there is provided a method of modulating activity of 11β-hydroxysteroid dehydrogenase type 1 (11β-HSD1) in a cell, the method comprising contacting the cell with 2-[(2,6-dimethyl-1-piperidinyl)carbonyl]-7-methyl-5-phenylpyrazolo[1,5-a]pyrimidine.

According to another aspect of the present application, there is provided a method of down-regulating activity of 11β-hydroxysteroid dehydrogenase type 1 (11β-HSD1) in a cell, the method comprising:

(A) contacting the cell with 2-[(2,6-dimethyl-1-piperidinyl)carbonyl]-7-methyl-5-phenylpyrazolo[1,5-a]pyrimidine; and

(b) analyzing activity of 11β-HSD1.

According to specific embodiments, the method comprising analyzing activity of 11β-HSD1.

Thus, according to another aspect of the present application, there is provided a method of modulating activity of 11β-hydroxysteroid dehydrogenase type 1 (11β-HSD1) in a cell, the method comprising:

(A) contacting the cell with 2-[(2,6-dimethyl-1-piperidinyl)carbonyl]-7-methyl-5-phenylpyrazolo[1,5-a]pyrimidine; and

(b) analyzing activity of 11β-HSD1.

“2-[(2,6-dimethyl-1-piperidinyl)carbonyl]-7-methyl-5-phenylpyrazolo[1,5-a]pyrimidine” can be obtained from e.g. ChemBridge (ChemBridge ID #5671423).

As used herein, the term “11β-hydroxysteroid dehydrogenase type 1 (11β-HSD1)”, refers to the polynucleotide or polypeptide expression product of the HSD11B1 gene (Gene ID: 3290). According to a specific embodiment, the 11β-HSD1 refers to the human 11β-HSD1, such as provided in the following Accession Numbers: NM_181755, NM_001206741, NM_005525, NP_001193670, NP_005516, NP_861420, NP_001193670, NP_005516 and NP_861420. According to a specific embodiment, the 11β-HSD1 refers to the mouse 11β-HSD1, such as provided in the following Accession Numbers: NM_001044751, NM_008288, NP_001038216 and NP_032314.

Analyzing activity of 11β-HSD1 may be effected by any method known in the art, including but not limited to the methods described in Cho et al., 2009; Solly et al., 2005; Schweizer et al., 2003, the contents of which are fully incorporated herein by reference.

The agents and compounds of some embodiments of the invention can be administered to a subject in combination with other established (e.g. gold standard) or experimental therapeutic regimen to treat a disease (e.g. a disease associated with mitochondrial dysfunction, oxidative stress and/or ER stress) including, but not limited to analgesics, chemotherapeutic agents, radiotherapeutic agents, cytotoxic therapies (conditioning), hormonal therapy, antibodies, antibiotics, anti-inflammatory drugs and other treatment regimens (e.g., surgery) which are well known in the art.

The agents and compounds of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent or the compound described herein accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent [e.g., production of a chimeric fusion protein that comprises a transport peptide that has an affinity for an endothelial cell surface molecule in combination with an agent that is itself incapable of crossing the blood brain barrier (BBB)] in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., leukodystrophy e.g. VWM) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient that are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In addition, the present inventors have developed a novel screening method.

Thus according to another aspect of the present invention there is provided a method of identifying an agent for the treatment of a disease associated with mitochondrial dysfunction, oxidative stress and/or ER stress, the method comprising determining a level of reactive oxygen species (ROS) in cells having an eIF2B deficiency following contacting with a test agent, wherein a decrease in the level of said ROS, as compared to same in the absence of said test agent, indicates efficiency of said test agent for the treatment of the disease.

According to specific embodiments of this aspect of the present invention, the contacting is effected in-vitro or ex-vivo.

As used herein the term “eIF2B deficiency” refers to reduced expression and/or activity of eIF2B as compared to a healthy control.

According to specific embodiments, the method is effected in-vitro or ex-vivo.

Determining a level of ROS may be effected by any method known in the art, including but not limited to, electron paramagnetic resonance spectroscopy (EPR), nuclear magnetic resonance (NMR), mass spectroscopy (MS), spectroscopy UV-Vis, gas chromatography (GC), chemiluminescence, HPLC-UV, iodide titration, ¹⁴C-formate oxidation and/or fluorescence e.g. cellROX, as further disclosed in the Examples section which follows.

According to specific embodiments, a significant decrease in the level of said ROS, as compared to same in the absence of said test agent, indicates efficiency of said test agent for the treatment of the disease.

According to specific embodiments, a decrease of at least 2%, at least 5%, at least 10%, at least 20%, at least 30%, at least 40%, at least 50%, at least 60%, at least 70%, at least 80%, at least 90%, or at least 99% e in the level of said ROS, as compared to same in the absence of said test agent, indicates efficiency of said test agent for the treatment of the disease.

According to specific embodiments, the method comprising determining survival of said cells following said contacting, wherein no statistically significant change in survival of said cells following said contacting as compared to survival in the absence of said test agent, indicates efficiency of said test agent for the treatment of the disease.

Determining survival may be effected by any method known in the art, including but not limited to MTT or crystal violet staining.

According to specific embodiments, the change in survival following said contacting is less than 20%, less than 10%, less than 5%, less than 2%, less than 1%, as compared to the survival in the absence of said test agent.

According to specific embodiments, the cells are human cells, such as e.g. from a biological sample of a subject having VWM disease.

According to specific embodiments, the cells are rodent cells (e.g. mouse)

According to specific embodiments, the cells are of Eif2b5^{R132H/R132H} mouse.

According to specific embodiments, the cells are genetically engineered to exhibit eIF2B deficiency.

The cells may be obtained from any gender and/or from any age.

According to specific embodiments, the cells are adult cells.

According to specific embodiments, the cells are postnatal cells.

According to specific embodiments, the cells are embryonic cells.

According to specific embodiments, the cells comprise fibroblasts or astrocytes.

According to specific embodiments, the cells comprise mouse embryonic fibroblasts (MEFs).

According to specific embodiments, the MEFs are of embryonic day E11-E17, E12-E16 or E13-E15.

According to a specific embodiments, the MEFs are of embryonic day E14.

According to specific embodiments, the cells are mouse astrocytes of post-natal day P0-P25, P0-P21, P0-P18, P0-P14, P0-P10, P0-P5 or P0-P2.

According to a specific embodiment, the cells are mouse astrocytes of post-natal day P0-P2.

It is expected that during the life of a patent maturing from this application many relevant Sig-1R agonists will be developed and the scope of the term “an agent capable of up-regulating activity of a component participating in a Sig-1R signaling pathway” is intended to include all such new technologies a priori.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Mice and Cells—

Wild-type (WT; C57BL strain) and Eif2b5^{R132H/R132H} (Mut; mutant) mice of both sexes were bred and housed in Tel Aviv University animal facility with 14/10 hours light/dark cycle in groups of four animals per cage in individually ventilated cages (Lab Products Inc., Seaford, Del., USA) supplemented with autoclaved wood chips. Animals were fed with autoclaved rodent pellet (Koffolk 19-510; Koffolk Ltd, Petach Tikva, Israel) and sterile water ad libitum. All experimental procedures were approved by the Tel Aviv University Animal Care Committee according to national guidelines (permits # L-15-037 and #04-12-27). Breeding and genotyping were performed as previously described (Geva et al., 2010). Each generation was established by back cross of homozygous Mut with WT C57BL/6J (Harlan Labs, Jerusalem, Israel) to prevent genetic drift. Primary cultures of fibroblasts from E14 embryos (MEFs) and astrocytes (from P0-P2 newborns) were isolated and used as previously describes (Raini et al., 2017). Primary Oligodendrocyte precursor cells (OPC) were isolated sequentially following astrocytes isolation using the anti-CD140α (PDGFRα) MicroBead Kit (Miltenyi Biotec 130-101-502). Shh-light2 cells (Taipale et al., 2000) were maintained in DMEM supplemented with 10% Fetal Bovine Serum, 100 U/mL penicillin, 0.1 mg/mL streptomycin, 2 mM L-glutamine, 400 μg/ml G418 (A.G. Scientific), and 200 μg/ml Zeocin (InvivoGen).

RNA and Protein Extractions from Brains—

Brains were removed from p14, P18 and P21 mice. Cerebrums were flash frozen in liquid nitrogen and kept in −80° C. until use. RNA was extracted from left hemispheres using RiboEX (GeneAll). Proteins were extracted from left hemispheres by sonication in 500 μl per hemisphere of lysis buffer containing 1% triton, 0.5% NaDOC, 0.1% SDS, 50 mM Tris pH 8, 100 mM NaCl, 10 mM β-Glycerophosphate, 5 mM NaF, 1 mM DTT, 1 mM Vanadate, and EDTA-free Complete™ protease inhibitor cocktail (#11-836-170-001; ROCHE). Following spinning for 15 minutes at 13,000 rpm, 4° C., the supernatant was analyzed for total concentration using BCA protein assay kit (#23227 Pierce).

Materials—

The compounds selected from DIVERSet™-EXP library and 4-[5-(3-methylphenoxy)pentyl]morpholine [designated herein as p8G5 or hit no. 8 (H8)]analogs were purchased from ChemBridge (San Diego, Calif., USA). SAG (#4366), Cyclopamine (#1623), pre-084 (#0589), and NE-100 (#3133) were purchased from Tocris Bioscience. Pridopidine (# M326195) was purchased from Toronto Research Chemicals (Toronto, Ontario, Canada), Tunicamycin (# T7765) was purchased from Sigma. All compounds were dissolved in DMSO, cyclopamine was dissolved in ethanol.

Selection of Screening Library and Clustering—

The algorithm for the selection of optimal screening libraries is described in details in (Gilad et al., 2015) and was followed with no modifications. For the purpose of clustering, each of the 50,000 compounds comprising the DIVERSet™-EXP library was first characterized by the ECFP6 fingerprints as implemented in BIOVIA's Discovery Studio Version 3.5. Following, the compounds were clustered into 500 clusters using the Hierarchical clustering as implemented in Schrodinger's Canvas (Canvas, Schrödinger, LLC, New York, N.Y., 2018). Finally, from each cluster, a compound closest to its center was selected for biological evaluation.

Docking—

All calculations were performed in BIOVIA's Discovery Studio Version 3.5. The crystal structure of Sigma-1-Receptor (Sig-1R) in complex with N-(1-benzylpiperidin-4-yl)-4-iodobenzamide (code 5HK2 in PDB protein data base) was retrieved from the PDB. Prior to docking, the protein structure was prepared using the prepare protein protocol. Docking was performed with the CDOCKER program using default parameters.

Image-Based Single Cell Analysis—

MEFs were seeded on 1% gelatin-coated 96-wells plate at a density of 5000 cells per well. 24 hours post-plating the cells were incubated with the tested compounds for additional 24 hours. Several DMSO-treated cells (control) were included in each plate at different locations. Following, the cells were stained by addition of fluorogenic dyes for further 30 minutes incubation at 37° C. Hoechst 33258 (#861405; Sigma-Aldrich) and JC-1 (# T4069; Sigma-Aldrich) were used at final concentration of 2 μg/ml; CellTrace CFSE (# C345545; Molecular Probes), 5 μM CellROX Deep Red (# C10422; Molecular Probes) at A final concentration of 5 μM. CellROX was used together with Hoechst and CFSE; JC1 was used together with Hoechst. Cells were washed with Hank's balanced salt solution (HBSS) which was used for images acquisition using IN Cell Analyzer 2000 (GE Healthcare, Pitsburgh, Pa., USA). IN Cell Developer Toolbox 1.9.1 software (GE Healthcare) was used for analysis which included cells segmentation using Hoechst and/or CFSE signals. For analysis of JC1 staining, integrated intensity of green and red emissions were used for detection of damaged and intact mitochondria, respectively.

Cell Survival Assay—

Cells were seeded on 96-wells plate at a density of 5000 cells per well. Astrocytes were seeded following coating with 0.001% PDL. 24 hours post-plating cells were incubated with the tested compounds for additional 24 hours followed by staining with 0.1% crystal violet/4% formaldehyde/1% ethanol as described in (Heiss et al., 2014).

Quantification of Gli1 mRNA—

Total RNA was reverse transcribed using qscript cDNA synthesis kit (#95047 Quanta Biosciences) and subjected to qPCR analysis using SYBR-Green (PerfeCTa® SYBR® Green FastMix®, ROX™; #95073; Quanta Biosciences) and the following oligonucleotide primers: Gli1 Fwd 5′-CCCATAGGGTCTCGGGTCTCAAAC-3′ (SEQ ID NO: 1) and Gli1 Rev 5′-GGAGGACCTGCGGCTGACTGTGTAA-3′ (SEQ ID NO: 2) for Gli1 mRNA amplification and Gapdh Fwd 5′-TGGCAAAGTGGAGATTGTTGCC-3′ (SEQ ID NO: 3) and Gapdh REV 5′-AAGATGGTGATGGGCTTCCCG-3′ (SEQ ID NO: 4) for Gapdh mRNA as an internal control. Reactions were carried out for 40 cycles in StepOne Real-time PCR apparatus (Applied Biosystems). Average relative quantity (RQ) was calculated by the ΔΔCt method.

Luciferase Activity Assay—

Shh-LIGHT2 cells were seeded at a density of 10,000 cells per well in a 96-wells plate in growing medium. 24 hours post-plating the cells were incubated for additional 24 hours with the tested compounds in low serum media (0.5%) without G418 and Zeocin. Following lysis, Firefly and Renilla luminescence was measured using the Dual Luciferase assay kit (Promega) and a Veritas microplate luminometer (Turner Biosystems).

Western Blot Analysis—

10⁵ Astrocytes or 1.5×10⁵ MEFs per well were seeded in a 6-wells plate and cultured for 3 or 2 days, respectively. Cells were washed with PBS, pelleted and resuspended in lysis buffer containing 1% Triton, 20 mM Tris pH=8.0, 100 mM KCl, 10% Glycerol, 10 mM β-Glycerophosphate, 50 mM NaF, 0.5 mM DTT, 1 mM Vanadate, and EDTA-free Complete™ protease inhibitor cocktail tablet from ROCHE (FIG. 1) or 1.6% SDS, 80 mM DTT, 8% Glycerol, 64 mM Tris pH=6.8, applied directly on the cell monolayer followed by extract collection (FIG. 16). Equal amounts of total cytoplasmic protein as assayed by Bradford protein assay (Bio-Rad) (FIG. 1) or equal volumes of total cell extract (FIG. 16) were separated by 15% SDS-PAGE followed by immunoblot analysis using antibodies specific for Sig-1R (abcam # ab53852 in FIG. 1 or Santa Cruz #137075 in FIG. 16), GAPDH (abcam # ab9485) (FIG. 1) and SDHB (Abcam # ab14714) (FIG. 16). The enhanced chemiluminiscence signal was captured using a AI600 Imager (Amersham) and quantified by ImageQuant TL (GE Healthcare).

mtDNA Quantification—

As detailed in (Raini et al., 2017).

TMRE Staining—

MEFs were seeded at a density of 4×10⁴ cells per well in a 24-wells plate. 24 hours post-plating the cells were incubated with the tested compounds for additional 6 hours followed by staining with 200 nM Tetra-Methyl-Rhodamine-Ethyl esterperchlorate (TMRE) (Abcam) for 30 minutes at 37° C. Cells were removed by trypsinyzation, washed and resuspended in PBS. 5-10×10³ cells were analyzed by Stratedigm S1000EXi cell sorter and FlowJo software (FLOWJO, LLC, Ashland, Oreg., USA).

Oxygen Consumption—

As detailed in (Raini et al., 2017) with the following modifications: oligomycin (Sigma, #04876) was used at concentrations of 1 μM for MEFs and 2 μM for astrocytes; FCCP (# C2920, Sigma) at 1.5 μM, antimycin A (# A8674, Sigma) at 0.5 μM for MEFs and 1 μM for astrocytes and rotenone (# R₈₈₇₅, Sigma) at 0.5 μM for MEFs and 1 μM for astrocytes. The data were normalized to cell number, obtained by Crystal Violet staining, or to mtDNA content.

OPC Differentiation Assay—

5,000 cells OPCs per well were seeded in a 96 wells plate and maintained overnight in proliferation media (Emery et al. 2013). Cells were then cultured for 60 hours in an IncuCyteZOOM instrument with differentiation medium (Emery et al. 2013) containing or not SAG at 50 nM or Cyclopamine at 1 μM. OPC differentiation was monitored by continuous imaging. Images taken at ten hours intervals were analyzed by IncuCyte NeuroTrack software for the following parameters: Neurite length, Neurite branch points, Cell body clusters and Cell body cluster area. Statistics—For all comparisons, Student's t-test was performed using ≥3 independent biological repeats for each group (methodological selection of sample size was not applied).

### Example 1

### Selection of Compounds as Candidates for the Treatment of Vwm Disease

A computational workflow for rational selection of an optimal screening library (Gilad et al., 2015) was applied to the analysis of nine commercially available screening libraries containing ˜380,000 compounds. The libraries were ranked based on three criteria: (1) ADME/T profiling including prediction of blood brain barrier permeability performed with a newly developed QSAR model; (2) Internal diversity; and (3) Similarity to the known active compound Guanabenz. All three criteria were combined into a single library score using a consensus approach while assigning equal weights to all criteria. The rankings based on the individual criteria and the consensus rankings are presented in Table 1 below. The results indicated that the DIVERSet™-EXP library by Chembridge is the best screening library for the current project.

The homozygous mouse model, Eif2b5^{R132H/R132H} (also referred to herein as Mut or mutant) is a mouse model of VWM disease has ˜20% decrease in brain eIF2B GEF activity leading to mild impairment of motor functions with involvement of white matter deficits. Thus, for screening, the differential cellular phenotypic characteristics of primary fibroblasts (MEFs) isolated from Eif2b5^{R132H/R132H} mouse model (Geva et al., 2010) was utilized and the assay was based on their abnormally high mitochondria content. This approach was chosen as an outcome of a previous study showing that increased mitochondrial biogenesis in eIF2B-mutant MEFs is a compensatory response to overcome compromised oxidative phosphorylation due to the mutation (Raini et al., 2017). It was hypothesized that incubation of the mutant cells with a compound that leads to enhanced mitochondrial function will alleviate their need to increase mitochondrial content. Since the cellular assay relies on small differences in mitochondrial function, it became apparent that medium throughput screening employing several repeats with several batches of cells is likely to give much better results than high throughput screening. This required reducing the number of compounds tested at each cycle. It was anticipated that experimental testing of a subset of compounds which well represents the entire parent library (i.e., a representative subset), should provide a similar amount of information as would be provided by testing the entire library. This hypothesis stems from the similar property principle, which states that similar compounds have similar properties. Therefore, the DIVERSet™-EXP library was clustered into 500 clusters and a single representative from each cluster, closest to the cluster center, was selected. Following biological testing, active hits could be easily traced back to their parent clusters and testing additional members of “active clusters” provided valuable SAR (Structure Activity Relationship) information.

To assay the compounds, cellROX, a reactive oxygen species (ROS) fluorescent detector and a preferred in-situ mitochondrial content detector, was used for single cell-based imaging analysis as detailed previously (Raini et al., 2017). Briefly, the median cellROX integrated intensity of non-treated cells was set as a threshold to define the population above it, as ‘high-ROS cells’. This fraction, being 50% in non-treated cells, was set as 1. If a compound acts via one of numerous possible mechanisms to decrease ROS levels, it was expected to be <1. A compound was considered a ‘hit’ if it was able to decrease the size of ‘high-ROS cells’ population to 0.9 or below, in at least three independent experiments. The first screening round identified eight hits, each representing a different cluster. In the second round, 437 compounds that were included in the eight relevant clusters were screened and 20 hits were identified, designated H1-H2O (FIG. 14A; see chemical names in Table 2 hereinbelow). Compounds toxicity was evaluated by testing cell survival following 24 hours incubation with 10 μM of each compound using crystal violet staining (FIG. 14B). Only H16 has led to a significant decrease in cell viability and therefore not used for further analyses. The other 19 compounds were not toxic; therefore, maximal working concentrations tolerated by the cells were further evaluated (Table 3 hereinbelow).

In the next step, in order to discover putative targets for the identified hits, the Scifinder® database was employed to search for structure similarities between the hits and compounds whose targets/signaling pathways are already known. Six hits were mapped to three putative targets/pathways, i.e., Sigma-1-Receptor (Sig-1R), Sonic hedgehog (Shh) and 11β-hydroxysteroid dehydrogenase type 1 (11β-HSD1), as further described in Examples 2-3 hereinbelow. These six hits were further tested for their ability to enhance mitochondrial health using the fluorescent dye JC1, the red and green emission of which reports, based on membrane potential, on intact and damaged mitochondria, respectively. Hits H8 (also designated herein as p8G5, putative target: Sig-1R), H15 (also designated herein as p9E6, putative target: Shh) and H17 (putative target: 11β-HSD1) elicited a significant beneficial effect on mitochondrial health as they led to either or both increased level of intact mitochondria and decreased level of damaged mitochondria (FIG. 14C).

TABLE 1
Library ranking

| | | ADME/T | Diversity | Similarity | Final |
|---|---|---|---|---|---|
| Library | Size | Ranking | Ranking | Ranking | Rank |
| Elite Libraries | 70,114 | 2 | 8 | 8 | 8 |
| Platinum | 113,962 | 6 | 7 | 5 | 8 |
| Collection | | | | | |
| DIVERSet ™-CL | 50,000 | 2 | 4 | 7 | 5 |
| DIVERSet ™- | 50,000 | 1 | 5 | 1 | 1 |
| EXP | | | | | |
| Drug-Like Set | 20,160 | 3 | 2 | 3 | 2 |
| Pharmacological | 10,240 | 3 | 6 | 6 | 6 |
| Diversity Set | | | | | |
| Maybridge | 54,318 | 4 | 3 | 2 | 3 |
| Screening | | | | | |
| Collection | | | | | |
| Prestwic | 1,280 | 7 | 1 | 9 | 7 |
| Chemical | | | | | |
| Library ® | | | | | |
| MSII Full Library | 10,000 | 5 | 2 | 4 | 4 |

TABLE 2
Hits and analogs

| ID | Chemical Name |
|---|---|
| Hits | |
| H1 | 5-benzyl-2-[(2-chlorophenyl)imino]-1,3-thiazolidin-4-one |
| H2 | 5-butyl-3-{[2-(4-morpholinyl)ethyl]thio}-5H-[1,2,4]triazino[5,6-b]indole |
| H3 | 2-phenyl-N′-({5-[3-(trifluoromethyl)phenyl]-2-furyl}methylene)acetohydrazide |
| H4 | 1-{3-[(4-chlorobenzyl)oxy]phenyl}ethanone |
| H5 | 1-allyl-2-(3,4,5-trimethoxyphenyl)-1H-benzimidazole |
| H6 | 7-(difluoromethyl)-N-[2-(4-morpholinyl)ethyl]-5-phenylpyrazolo[1,5- |
| | a]pyrimidine-3-carboxamide |
| H7 | 1-phenyl-4-[4-(2-thienylcarbonyl)-1-piperazinyl]phthalazine |
| H8 (p8G5) | 4-[5-(3-methylphenoxy)pentyl]morpholine |
| H9 | 1-(2-fluorophenyl)-4-(phenylacetyl)piperazine |
| H10 | 2-{[2-oxo-2-(1-piperidinyl)ethyl]thio}-4-phenyl-6-(trifluoromethyl)pyrimidine |
| H11 | N-[2-(phenylthio)cyclohexyl]benzenesulfonamide |
| H12 | 1-{[3-(benzyloxy)phenyl]carbonothioyl}-4-methylpiperazine |
| H13 | 5-phenyl-N-(2-thienylmethyl)-7-(trifluoromethyl)pyrazolo[1,5-a]pyrimidine-2- |
| | carboxamide |
| H14 | 2-(2,3-dihydro-9H-imidazo[1,2-a]benzimidazol-9-yl)-1-(4- |
| | hydroxyphenyl)ethanone hydrobromide |
| H15 (p9E6) | 1-allyl-2-(2-phenylvinyl)-1H-benzimidazole |
| H16 | 2-[(2,5-dimethoxyphenyl)diazenyl]-1-methyl-1H-benzimidazole |
| H17 | 2-[(2,6-dimethyl-1-piperidinyl)carbonyl]-7-methyl-5-phenylpyrazolo[1,5- |
| | a]pyrimidine |
| H18 | 2-(4-morpholinylmethyl)-1-(1-naphthylmethyl)-1H-benzimidazole |
| H19 | 2-(benzylthio)-N-cyclopentylbenzamide |
| H20 | 5-isopropyl-N-methyl-3-phenylpyrazolo[1,5-a]pyrimidin-7-amine |
| Analogs | |
| H8-1 | 4-[5-(3,5-dimethylphenoxy)pentyl]morpholine |
| H8-2 | 4-[5-(3,4-dimethylphenoxy)pentyl]morpholine |
| H8-3 | 4-[6-(3-methylphenoxy)hexyl]morpholine |
| H8-4 | 4-[4-(3-methylphenoxy)butyl]morpholine |
| H8-5 | 4-[4-(3,4-dimethylphenoxy)butyl]morpholine |
| H8-6 | 4-[5-(3-methoxyphenoxy)pentyl]morpholine |
| H8-7 | 4-[5-(3-chlorophenoxy)pentyl]morpholine |
| H8-8 | 1-[5-(2-fluorophenoxy)pentyl]-4-methylpiperazine |

TABLE 3
Effect of compounds on cell survival evaluated by Crystal violet staining

| | | | Survival (%) ± SEM | | |
|---|---|---|---|---|---|
| | | μM | Mut MEFs | WT astrocytes | Mut astrocytes |
| Sig1R binders | pre084 | 0.1 | 100 ± 2.1 | | |
| | | 1 | 101 ± 3.5 | 114 ± 7.9 | 97 ± 0.5 |
| | | 10 | 99 ± 1.5 | 95 ± 2.3 | 93 ± 5.1 |
| | | 20 | 97 ± 2.1 | | |

| | | | | | |
|---|---|---|---|---|---|
| 30 | 105 ± 1.9 | 82 ± 1.9 | (p = 8.3E−04) | 76 ± 4.9 | (p = 0.03) |

| | | |
|---|---|---|
| 50 | 80 ± 2.8 | (p = 0.01) |
| 100 | 57 ± 2.2 | (p =1.5E−05) |

| | | | | |
|---|---|---|---|---|
| pridopidine | 0.1 | 102 ± 1.7 | | |
| | 1 | 103 ± 1.3 | 103 ± 3.5 | 97 ± 2.7 |
| | 10 | 98 ± 1.6 | 101 ± 2.2 | 92 ± 2.8 |
| | 20 | 99 ± 0.64 | | |

| | | | | |
|---|---|---|---|---|
| 30 | 99 ± 3.1 | 85 ± 4.3 | (p = 0.02) | 97 ± 5.0 |

| 50 | 97 ± 1.2 | |
|---|---|---|
| 100 | 91 ± 2.4 | (p = 0.03) |

| | | | | |
|---|---|---|---|---|
| H8 (p8G5) | 0.1 | 104 ± 4.6 | | |
| | 1 | 102 ± 3.3 | 132 ± 10.7 | 108 ± 0.6 |
| | 10 | 99 ± 2.6 | 102 ± 3.8 | 93 ± 2.1 |

20

| | | | | |
|---|---|---|---|---|
| 30 | 105 ± 13 | 99 ± 3.2 | 92 ± 1.6 | (p = 0.14) |

| | | | |
|---|---|---|---|
| | 50 | | |
| | 100 | | |
| NE100 | 0.1 | 99 ± 2.1 | 98 ± 1.8 |

| 1 | 98 ± 2.2 | | 86 ± 1.1 | (p = 1.5E−5) | 94 ± 4.7 | |
|---|---|---|---|---|---|---|
| 10 | 88 ± 3.5 | (p = 0.017) | 79 ± 2.2 | (p = 1.6E−4) | 82 ± 4.3 | (p = 0.01) |

| | | |
|---|---|---|
| 30 | 65 ± 2.2 | (p =6.0E−06) |

| | | | |
|---|---|---|---|
| H8 analogs | H8-1 | 10 | 104 ± 2.7 |
| | | 20 | 103 ± 4.7 |
| | | 30 | 97 ± 3.9 |
| | H8-2 | 10 | 92 ± 4.2 |
| | | 20 | 94 ± 8.8 |
| | | 30 | 95 ± 13.1 |
| | H8-3 | 10 | 100 ± 7.2 |
| | | 20 | 96 ± 4.1 |

| | | |
|---|---|---|
| 30 | 88 ± 4.5 | (p = 0.09) |

| | | |
|---|---|---|
| H8-4 | 10 | 92 ± 8.5 |
| | 20 | 95 ± 4.2 |
| | 30 | 98 ± 5.4 |
| H8-5 | 10 | 92 ± 1.7 |
| | 20 | 100 ± 4.4 |
| | 30 | 98 ± 13.8 |
| H8-6 | 10 | 97 ± 5.1 |
| | 20 | 92 ± 1.1 |
| | 30 | 93 ± 5.2 |
| H8-7 | 10 | 98 ± 5.1 |
| | 20 | 91 ± 4.7 |

| | | |
|---|---|---|
| 30 | 88 ± 9.7 | (p = 0.44) |

| | | | |
|---|---|---|---|
| | H8-8 | 10 | 101 ± 5.6 |
| | | 20 | 99 ± 11 |
| | | 30 | 90 ± 3.5 |
| Hedgehog | SAG | 0.01 | 98 ± 2.1 |
| modulators | | 0.05 | 92 ± 3.2 |
| | | 0.1 | 95 ± 2.1 |

| | | |
|---|---|---|
| 1 | 68 ± 2.7 | (p =1.9E−05) |

| | | |
|---|---|---|
| Cyclopamine | 0.1 | 92 ± 2.5 |
| | 1 | 95 ± 4.1 |

| | | |
|---|---|---|
| 10 | 79 ± 1.4 | (p = 0.02) |

*Cell survival of untreated cells was set to 100%.
Shown is % cell survival ± SEM of at least 3 repeats.
Toxicity refers to survival rate below 90%.

### Example 2

### Sig-1R Agonists for the Treatment of VWM Disease

### Relevance of Sigma-1 Receptor (Sig-1R) as a Target for VWM Treatment

As described in Example 1 hereinabove, the differential cellular phenotypic characteristics of primary fibroblasts (MEFs) isolated from the Eif2b5^{R132H/R132H} mouse model was utilized for the development of a novel Image based assay for screening of repair effects of drug-like compounds. The assay was based on the increased ROS levels in mutant MEFs due to compromised mitochondria function. The DIVERSet™-EXP library (ChemBridge) was screened based on computer-aided considerations and out of 50,000 compounds a compound—4-[5-(3-methylphenoxy)pentyl]morpholine [hit no. 8 (H8), designated herein as p8G5] was selected.

p8G5 Binds to Sig-1R

Using SciFinder structure similarities between p8G5 and a known Sig-1R binder were detected and therefore it was hypothesized that p8G5 is also a Sig-1R binder.

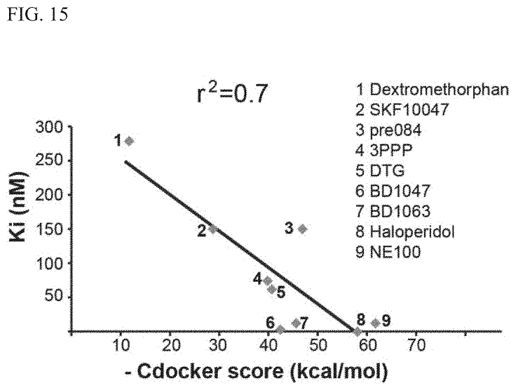

To this end, computational analysis was used to evaluated whether p8G5 (H8) is indeed a potential binder of Sig-1R by docking simulations using the CDOCKER program (Wu et al., 2003) as implemented in BIOVIA's Discovery Studio (BIOVIA, 2016). Initially it was tested whether CDOCKER can reliably reproduce the ligand binding mode of a known binder, using the crystal structure of Sig-1R in complex with N-(1-benzylpiperidin-4-yl)-4-iodobenzamide (code 5HK2 in the PDB protein data bank). Indeed, the lowest energy-binding mode obtained with CDOCKER had a root mean square value (RMSD) of 0.8 Å with respect to the crystal structure, suggesting that the program is appropriate for this study. Next, nine Sig-1R binders with known ligand-protein binding Ki values were retrieved from PubChem (www(dot)pubchem(dot)ncbi(dot)nlm(dot)nih(dot)gov) and docked into the Sig-1R structure. The scores of the top ranked pose of each ligand correlated with the experimental Ki values and a significant correlation with r²=0.7 was obtained (FIG. 15). Following, p8G5 (H8) was docked into this structure and its score was found to be 49.38 kcal/mol, which is within the range of scores for the known binders.

Following, the direct binding of p8G5 to Sig-1R, was tested in-vitro by Eurofins labs (www(dot)cerep(dot)fr/cerep/users/pages/catalog/Affiche_CondExp_Test(dot)asp?test=889).

A competitive displacement binding assay employing the known binder [³H]haloperidol (Ganapathy et al. 1999) revealed that 10 μM of p8G5 inhibited 92% of Haloperidol binding, confirming that it is a direct Sig-1R binder.

Sig-1R protein expression level is decreased in cells derived from eIF2B5^{R13211/R132H} mice Sig-1R is a chaperone protein located at the ER-mitochondria interface, that regulates ER and mitochondrial function and crosstalk (Su et al. 2016) (Bernard-Marissal et al. 2015). Among its many functions, it ensures Ca²⁺ signaling from the ER into the mitochondria by chaperoning IP3 receptor (Hayashi and Su, 2007). Its role is important for multiple cellular scenarios including astrocytes activation (Zhang et al. 2015) and oligodendrocyte proliferation, differentiation and myelin production (Demerens et al. 1999, Lisak et al. 2014).

Sig-1R expression was tested by western blot analysis in primary mouse embryonic fibroblasts (MEFs) and primary brain astrocytes isolated from eIF2B5^{R13211/R132H} (Mut) and wild-type C57BL (WT) mice. Mut MEFs and Mut astrocytes express ˜60% and −20% of Sig-1R protein level compared to WT MEFs and astrocytes, respectively (FIG. 1).

Primary MEFs and astrocytes isolated from Eif2b5^{R132H/R132H} mice suffer from oxidative respiration defects, leading to increased mitochondrial biogenesis for compensation purposes in order to meet energetic needs (Raini et al., 2017). It was therefore important to test Sig-1R protein level per mitochondria. For this purpose, the ratio between Sig-1R protein level and the level of the nuclear-encoded SDHB protein, a component of the mitochondrial electron transfer chain (ETC) complex, was tested. Western blot analysis revealed that primary MEFs and astrocytes isolated from mutant mice express 17% and 22% lower S1R/SDHB protein ratio compared to WT MEFs and astrocytes, respectively (FIGS. 16A-B). Brain extracts of mice at postnatal ages of P14-P18 showed S1R/SDHB levels that are lower by 34-35% compared to WT controls (FIG. 16C).

Considering that astrocytes play a key role in supporting oligodendrocyte function and that astrocytes have a key role in VWM disease (Dooves et al. 2016), these results point to Sig-1R as a highly relevant target for the treatment of VWM disease.

Sig-1R Involvement in Correcting the Abnormal High Levels of Mitochondrial Content and Reactive Oxygen Species (ROS) in Mut Cells

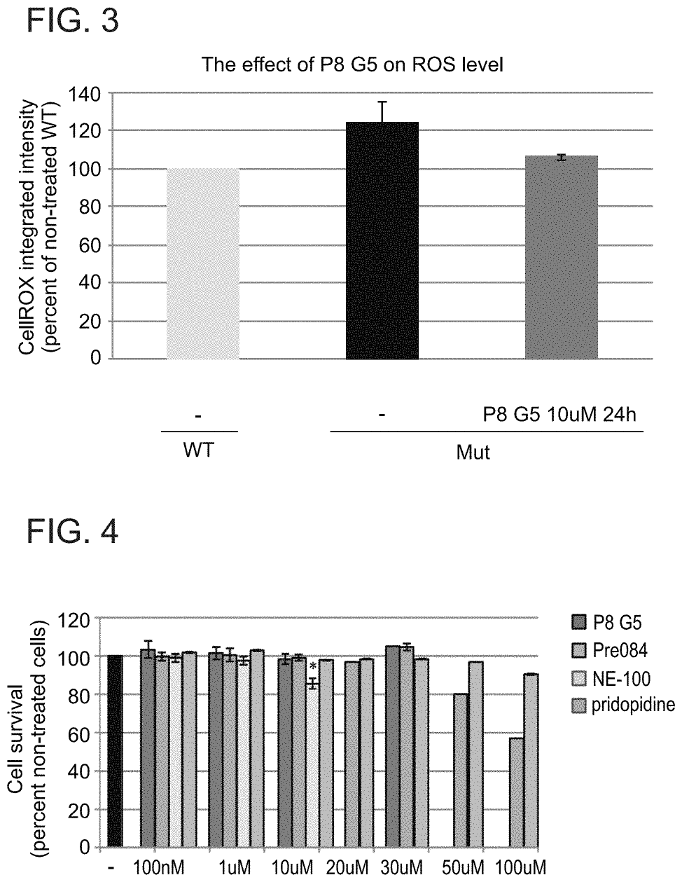

As noted above, eIF2B5^{R13211/R132H} mice suffer from oxidative respiration defects, leading to a compensation response resulting in increased mitochondrial biogenesis to meet energetic needs (Raini et al, 2017). Hence, the effect of targeting Sig-1R on mitochondrial content was evaluated. A short treatment (6 hours incubation) with 0.1 μM-1 μM of p8G5 or Pre084 (a known Sig-1R agonist) or 1 μM-10 μM of pridopidine (a known Sig-1R agonist) lowered the abnormal high mitochondrial content in Mut MEFs and brought it closer to the WT level (FIG. 2). On the contrary, NE-100 (a known Sig-1R antagonist) increased mitochondrial content in a dose dependent manner. Importantly, longer treatment (24 hours incubation) with 10 μM of p8G5 corrected the abnormal level of ROS in Mut cells by bringing it to WT level (FIG. 3). To rule out a decreased mitochondrial content and ROS levels due to a toxic effect, a survival assay was performed. Thus, incubation with as high as 30 μM p8G5, Pre084 or pridopidine did not have any negative effect on the survival of Mut cells (FIG. 4 and Table 3 hereinbelow.

### Positive Effect of Some Sig-1R Binders on Mitochondrial Function in Mut Cells

### 1) Increase of Mitochondrial Membrane Potential

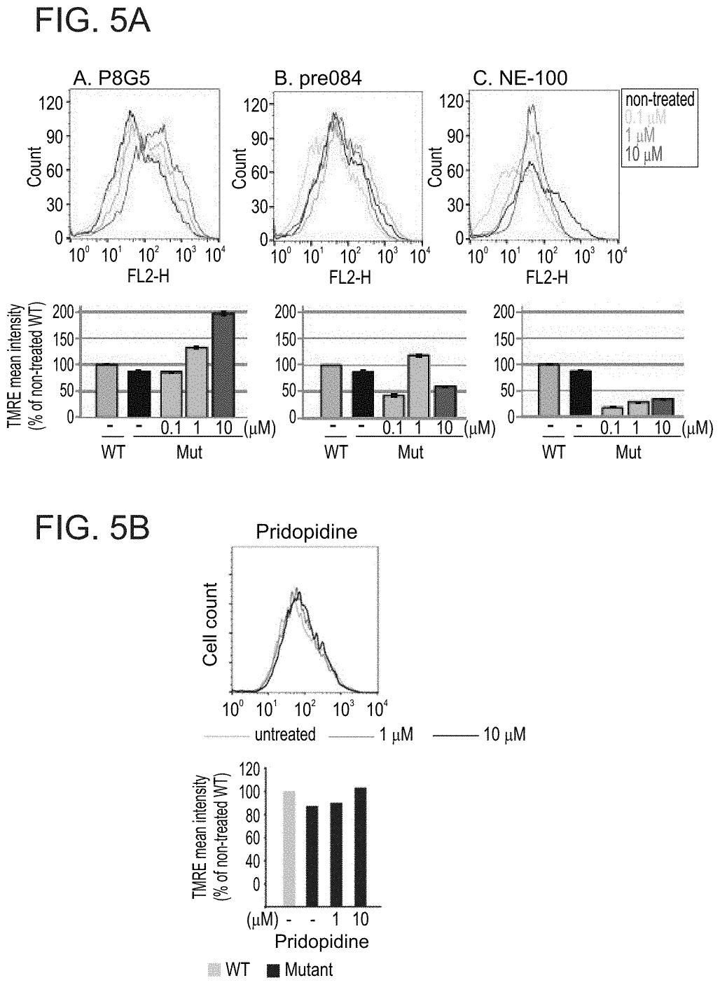

TMRE is a positively charged fluorescent dye specific for active mitochondria with membrane potential above a certain threshold. Incubation of Mut MEFs for 6 hours with 1 μM of p8G5 or pre084 (a known agonist) or 10 μM pridopidine increased TMRE staining to a level above that of untreated WT cells (FIGS. 5A-B). On the contrary, the known antagonist NE-100 had a negative effect on TMRE staining of Mut MEFs (thus on their mitochondrial membrane potential) at all concentrations (FIG. 5A).

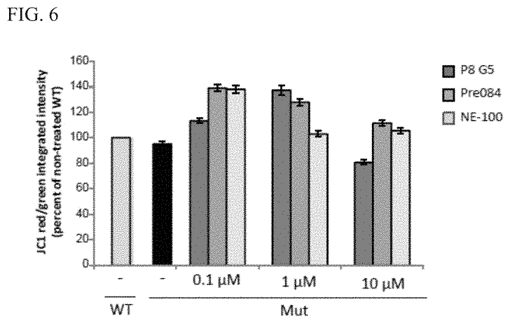

The increased TMRE staining may be the outcome of increased mitochondrial content or increased mitochondrial membrane potential, or both. As 1 μM and 10 μM of p8G5 and pre084 lowered the level of mitochondrial content (FIG. 2), the TMRE staining results indicate they positively affect membrane potential. To verify this important point, the JC1 fluorescent dye was used. The JC1 fluorescent dye stains in red the active mitochondria (high membrane potential) and in green the less active mitochondria (low membrane potential). As shown in FIG. 6, incubation with 1 μM and 10 μM of p8G5 or pre084 increased the ratio between the content of mitochondria with high to low membrane potential, as indicated by the increased red/green JC1 signal, indicating that activation of Sig-1R enhances mitochondrial health in Mut cells (FIG. 6), thus making it an attractive target for the treatment of VWM disease.

### 2) Increase of Oxidative Phosphorylation

The ultimate outcome of increased mitochondrial membrane potential is increased rate of oxidative phosphorylation and ATP-linked respiration.

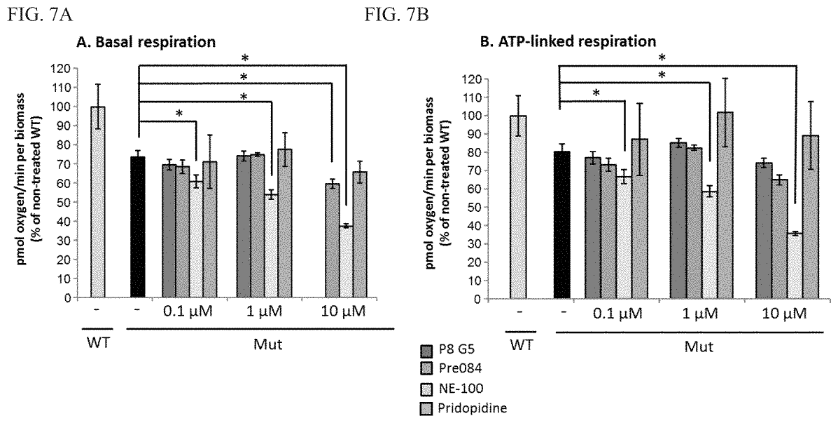

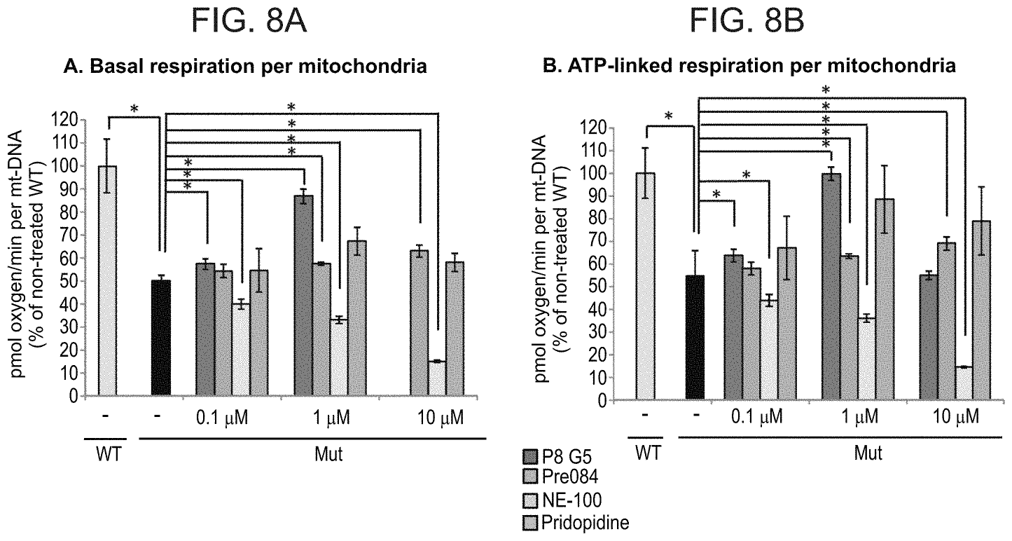

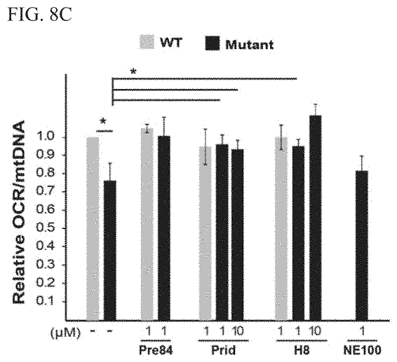

Measurement of basal respiration and ATP-linked respiration per cell, as measured by oxygen consumption rate (OCR) using the mito-stress kit (Agilent Technologies) revealed a negative effect by the Sig-1R antagonist NE-100, and no effect by the agonist pre084 or p8G5 (FIGS. 7A-B). However, given that the latter compounds lead to decreased mitochondrial content (FIG. 2) and increased mitochondrial potential (FIGS. 5 and 6), normalization of the respiration data to mitochondrial content revealed a statistically significant increase in basal and ATP-linked respiration rate per mitochondrial DNA content in Mut cells following incubation with pre084 or p8G5 compounds (FIGS. 8A-B). Thus, for example, incubation with 1 μM p8G5 for 6 hours brought both parameters to WT levels (FIGS. 8A-B). As further shown in FIGS. 8(A-B), Pridopidine as well elicited a beneficial effect on basal and ATP-linked respiration rate. In addition, determining maximal respiration rate per mitochondrial content (FIG. 8C) indicated that p8G5 (H8), pre084 and pridopidine at 1 μM increased the maximal respiration capacity per mitochondria to the level of untreated WT cells, whereas WT cells were not affected.

### 4) Effect on Primary Astrocytes

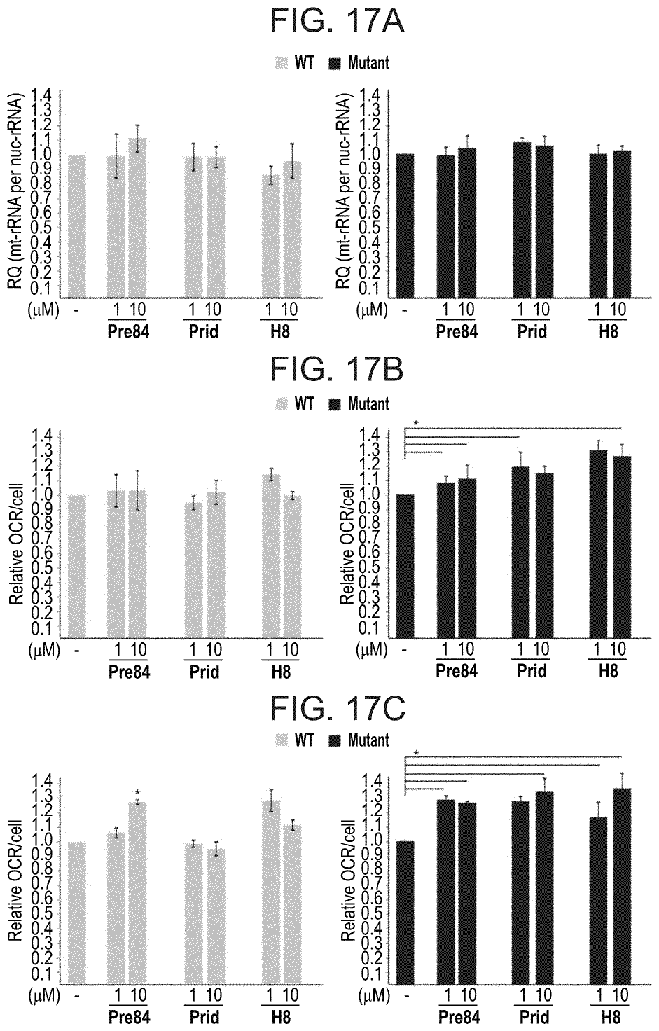

Due to the specific involvement of astrocytes in VWM disease, it was important to test the effect of Sig-1R agonists on primary astrocytes isolated from Eif2b5^{R132H/R132H} mutant mice (FIGS. 17A-C). Interestingly, while 1 or 10 μm Pre-084, pridopidine, or p8G5 (H8) increased both ATP-linked and maximal respiration in mutant astrocytes (FIGS. 17B-C), no effect on mitochondrial content per cell was observed (FIG. 17A). This phenomenon is consistent with the beneficial outcome of Sig-1R agonists, yet the partial correction of the anomaly in astrocytes is consistent with the high energetic requirements of these cells.

### Positive Effect of Sig-1R Binders on Survival of Mut MEFS Under ER-Stress Conditions

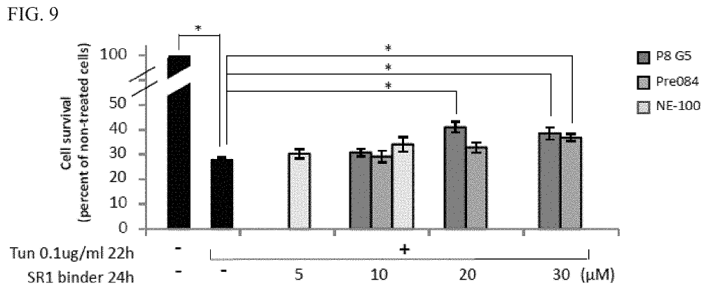

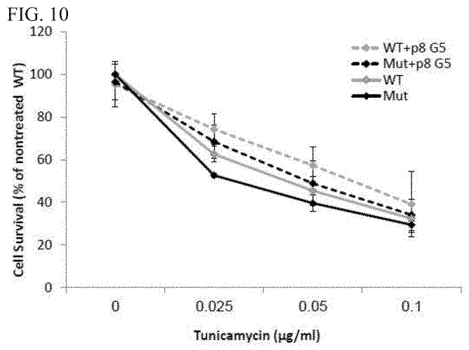

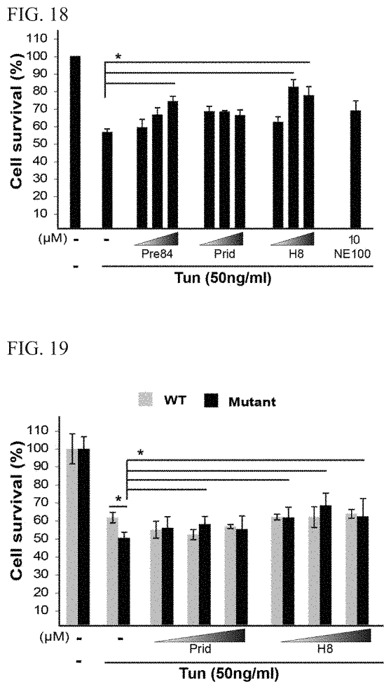

One of the known features of eIF2B-mutant cells is their hyper-sensitivity to ER-stress (Kantor et al. 2005, Kantor et al. 2008, Horzinski et al. 2010) (van der Voorn et al. 2005, van Kollenburg et al. 2006). The hypersensitivity of MEFs isolated from Mut mice was confirmed by their lower survival rate compared to WT controls upon 22 hours incubation with the ER stress agent Tunicamycin (Tun), as assayed by crystal violet staining (FIG. 10). Following, the effect of the Sig-1R binders on cell survival under ER-stress conditions was evaluated. Incubation of Mut MEFs with 20 μM of p8G5 (H8) attenuated the apoptotic effect of prolonged incubation with the ER-stress agent Tunicamycin (FIGS. 9, 10 and 18). 30 μM Pre084 was also able to rescue mutant MEFs from Tun-induced ER stress mediated cell death (FIGS. 9 and 18). In addition, primary astrocytes isolated from Mut mice exhibited hypersensitivity to ER stress compared to astrocytes isolated from WT controls mice. Furthermore, 30 μM pridopidine and 20-50 μM p8G5 (H8) increased the survival rate of Mut astrocytes under ER-stress conditions (FIG. 19).

Taken together, the results indicate that Sig-1R agonists[i.e. p8G5 (H8), Pre084 and pridopidine) are able to increase mitochondrial membrane potential and effective oxidative respiration of eIF2B-mutant cells; and to increase their ability to cope with chronic ER stress. Together with the finding that Sig-1R expression level is low in eIF2B-mutant MEFs and primary astrocytes, compounds acting as Sig-1R agonists[e.g. p8G5 (H8), Pre084 and Pridopidine] can be used for the treatment of VWM disease.

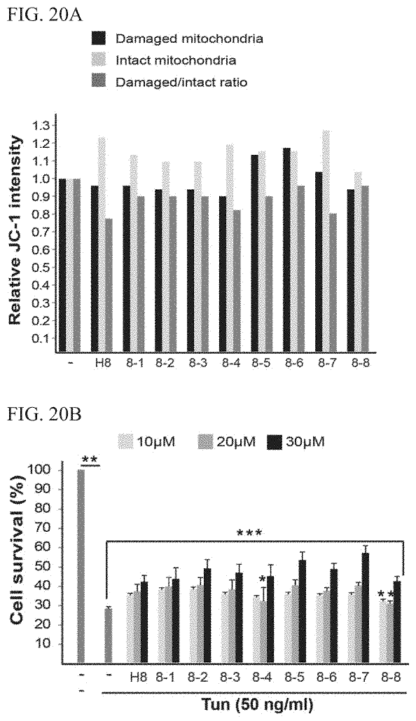

To find a more compounds based on the current study, several structural analogues of p8G5 (H8) were identified using structure similarity search. For this purpose, other EnamineStore and Chembridge libraries were scanned for compounds with structure similarity to that of p8G5 (H8). Eight similar analogues (listed in Table 2 hereinabove) were tested for their effect on mitochondrial health and cell survival following Tun-induced cell death. Most of the analogs elicited beneficial effect on mitochondrial health, causing decrease in damaged/intact organelle ratio, with analogue H8-7 at the top of the list (FIG. 20A). In addition, all the analogs exhibited a similar or better effect compared to H8, as all increased cell survival (FIG. 20B). Specifically, analogs H8-2, H8-5, H8-6 and H8-7 displayed the most potent effect at a concentration of 30 μM concentration (FIG. 20B).

### Example 3

### SHH as a Target for the Treatment of VWM Disease

### Relevance of Sonic Hedgehog (SHH) as a Target for VWM Treatment

As described in Example 1 hereinabove, the differential cellular phenotypic characteristics of primary fibroblasts (MEFs) isolated from the Eif2b5^{R132H/R132H} mouse model was utilized for the development of a novel Image based assay for screening of repair effects of drug-like compounds. The assay was based on the increased ROS levels in mutant MEFs due to compromised mitochondria function as described above. The DIVERSet™-EXP library (ChemBridge) was screened based on computer-aided considerations and out of 50,000 three compounds were selected: 1-allyl-2-(3,4,5-trimethoxyphenyl)-1H-benzimidazole (hit no. 5 (H5), designated herein as p8C10), 1-(2-fluorophenyl)-4-(phenylacetyl)piperazine (hit no. 9 (H9), designated herein as p8G7) and 1-allyl-2-(2-phenylvinyl)-1H-benzimidazole (hit no. 15 (H15) designated herein as p9E6).

p8C10, p8G7 and p9E6 Modulate SHH Signaling Pathway

Using SciFinder structure similarities between p8C10 (H5), p8G7 (H9), p9E6 (H15) and a known SHH signaling pathway modulator were found and therefore it was hypothesized that the selected compounds are also SHH modulators. Of note, an additional SHH modulator was also identified in the screening assay—1-phenyl-4-[4-(2-thienylcarbonyl)-1-piperazinyl]phthalazine (H7).

The SHH signaling pathway plays a central role in oligodendrocyte proliferation/differentiation and in myelin production (Arnett et al. 2004; Yoshimura and Takeda 2012; Samanta et al., 2015). SHH pathway is most important during brain development. Its activity increases to induce the first steps of oligodendrocyte proliferation and maturation followed by a subsequent decrease to allow complete differentiation and myelination (Bouslama-Queghlani et al., 2012). The expression of Gli1 transcription factor which is driven by SHH-signaling, is considered a sensitive readout of activation of this pathway (Dessaud et al., 2008). The mobilization of endogenous neural stem cells for remyelination by pharmacological inhibition of Gli1 has opened a new therapeutic avenue for the treatment of demyelinating disorders (Samanta et al., 2015 and International Patent Application Publication No. WO2013112859). SHH activity is also linked to mitochondrial health (Wu et al. 2009; Kwon et al. 2015; Malhotra et al. 2016).

To test if p8C10 (H5), p8G7 (H9) and p9E6 (H15) compounds that were identified in the screen indeed affect Hedgehog signaling, SHH-lightII cells which stably express an inducible Firefly-Luciferase reporter gene under the control of Gli1 (a read-out of Hedgehog activation) and constitutive Renilla-Luciferase for normalization purposes (Taipale et al. 2000) were used. It was found that only compound p9E6 (H15) was able to activate the Hedgehog signaling pathway, leading to increased Firefly/Renilla luciferase expression in a concentration dependent manner (FIG. 11A). Therefore, p9E6 was identified as an agonist. SHH activation is required for the first steps of oligodendrocyte differentiation, while a subsequent decrease in Hedgehog activity is needed for complete differentiation and myelin production (Bouslama-Oueghlani et al. 2012) and re-myelination (Samanta et al. 2015). To test whether the other two compounds that were not able to activate the pathway might act as antagonist, a known potent agonist, termed Smoothened agonist (SAG) was used and the ability of the compounds to counteract its activity was tested. p8C10 (H5) and p8G7 (H9) inhibited the SAG-induced HH activity (FIG. 11B), confirming their effect on this signaling pathway.

Hedgehog Pathway Regulation is Impaired in eIF2B5-Mutant Mice Brains

To test Hedgehog activity the mRNA level of its target gene Gli1 was measured by RT-qPCR using brain extracts from WT and mutant mice at the age of P18 and P21, the peak of oligodendrocyte differentiation and myelin formation. FIG. 11C shows that Hedgehog activity was significantly higher at P18 and lower at P21 in mutant compared to WT brains. Moreover, the normal expected decrease in Hedgehog activity between P18-P21 observed in WT brains was more prominent in mutants brains. The higher activity observed in P18 mutants may indicate delayed decrease in Hedgehog activity due to impaired programming of this pathway due to the mutation in eIF2B. This result is consistent with previous finding related to delayed waves of gene expression during brain development in mutants (Marom et al., 2011). These results bring up the role of Hedgehog pathway in VWM pathogenesis and illuminate it as a potential target for the treatment of VWM disease.

A Known SHH Antagonist has a Positive Impact on Mitochondrial Health in eIF2B-Mutant MEFs and on Differentiation Oligodendrocytes

As a proof of concept which demonstrates that modulation of SHH activity can reverse the abnormal mitochondria-related phenotype of eIF2B-mutant MEFs, the effect of Cyclopamine, a known SHH inhibitor, on the mitochondria was tested using JC-1 staining. Cyclopamine lead to increased content of intact mitochondria, decreased content of damaged mitochondria and decreased ratio of damaged to intact mitochondria (FIG. 12).

To assess the effect of SHH antagonist and agonist on the differentiation of oligodendrocytes precursor cells (OPC), primary OPCs were isolated from the brain of newborn mice and cultured in differentiation medium in the absence or presence of SAG or cyclopamine for 60 hours using a IncuCyte live cell analysis system. FIG. 13 shows the positive effect of cyclopamine on neurite length, neurite branch points, cell body clusters and cell body cluster area.

Taken together, the results show that indeed inhibition of SHH activity has a positive impact on mitochondrial health in eIF2B-mutant MEFs, and on differentiation of OPC to mature oligodendrocytes; illuminating the SHH pathway as a promising target for the development of a therapeutic strategy for VWM disease.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Other References are Cited Throughout the Application

Arnett, H. A., S. P. Fancy, J. A. Alberta, C. Zhao, S. R. Plant, S. Kaing, C. S. Raine, D. H. Rowitch, R. J. Franklin and C. D. Stiles (2004). “bHLH transcription factor Olig1 is required to repair demyelinated lesions in the CNS.” Science 306(5704): 2111-2115.
Bernard-Marissal, N., Medard, J. J., Azzedine, H. and Chrast, R. (2015) Dysfunction in endoplasmic reticulum-mitochondria crosstalk underlies SIGMAR1 loss of function mediated motor neuron degeneration. Brain, 138, 875-890.
BIOVIA, D. S. (2016). San Diego, Discovery Studio Modeling Environment. Release 2017.
Bouslama-Oueghlani, L., R. Wehrle, M. Doulazmi, X. R. Chen, F. Jaudon, Y. Lemaigre-Dubreuil, I. Rivals, C. Sotelo and I. Dusart (2012). “Purkinje cell maturation participates in the control of oligodendrocyte differentiation: role of sonic hedgehog and vitronectin.” PLoS One 7(11): e49015.Bugiani, M., Boor, I., Powers, J. M., Scheper, G. C. and van der Knaap, M. S. (2010) Leukoencephalopathy with vanishing white matter: a review. J Neuropathol Exp Neurol, 69, 987-996.
Brookes, P. S., Yoon, Y., Robotham, J. L., Anders, M. W. and Sheu, S. S. (2004). Calcium, ATP, and ROS: a mitochondrial love-hate triangle. Am J Physiol Cell Physiol 287(4), C817-833. DOI: 10.1152/ajpcell.00139.2004.
Cabilly, Y., Barbi, M., Geva, M., Marom, L., Chetrit, D., Ehrlich, M. and Elroy-Stein, 0. (2012) Poor cerebral inflammatory response in eIF2B knock-in mice: implications for the aetiology of vanishing white matter disease. PLoS One, 7, e46715.
Cho, Y. S., Kim, C. H. and Cheon, H. G. (2009). Cell-Based Assay for Screening 11β-Hydroxysteroid Dehydrogenase 1 Inhibitors. Analytical Biochemistry 392(2): 11β-116.DOI: www(dot)doi(dot)org/10(dot)₁₀₁₆/j(dot)ab(dot)₂₀₀₉(dot)₀₄(dot)₀₃₈.
Crowley, L. C., Christensen, M. E. and Waterhouse, N. J. (2016). Measuring Mitochondrial Transmembrane Potential by TMRE Staining. Cold Spring Harb Protoc 2016(12), pdb prot087361. DOI: 10.1101/pdb.prot087361.Demerens, C., Stankoff, B., Zalc, B. and Lubetzki, C. (1999) Eliprodil stimulates CNS myelination: new prospects for multiple sclerosis? Neurology, 52, 346-350.
Dessaud E, McMahon A P, Briscoe J. Pattern formation in the vertebrate neural tube: a sonic hedgehog morphogen-regulated transcriptional network. (2008) Development 135:2489-2503.
Dooves, S., Bugiani, M., Postma, N. L. et al. (2016) Astrocytes are central in the pathomechanisms of vanishing white matter. J Clin Invest, 126, 1512-1524.
Elroy-Stein, O., Schiffmann, R. (2015) Vanishing White Matter Disease. In: Rosenberg's Molecular and Genetic Basis of Neurological and Psychiatric Disease, (R. N. Rosenberg, Pascual, J. M. ed.), pp. 1015-1030. Elsevier Inc., London UK.
Elroy-Stein, O. (2017). Mitochondrial malfunction in vanishing white matter disease: a disease of the cytosolic translation machinery. Neural Regen Res 12(10), 1610-1612. DOI: 10.4103/1673-5374.217329.
Emery B and Dugas J C (2013). Purification of oligodendrocyte lineage cells from mouse cortices by immunopanning. Cold Spring Harb Protoc, 854-868.
Fogli, A. and Boespflug-Tanguy, O. (2006) The large spectrum of eIF2B-related diseases. Biochem Soc Trans, 34, 22-29.
Fuccillo, M., Joyner, A. L. and Fishell, G. (2006). Morphogen to mitogen: the multiple roles of hedgehog signalling in vertebrate neural development. Nat Rev Neurosci 7(10), 772-783. DOI: 10.1038/nrn1990.
Ganapathy, M. E., Prasad, P. D., Huang, W., Seth, P., Leibach, F. H. and Ganapathy, V. (1999) Molecular and ligand-binding characterization of the sigma-receptor in the Jurkat human T lymphocyte cell line. J Pharmacol Exp Ther, 289, 251-260.
Gat-Viks, I., Geiger, T., Barbi, M., Raini, G. and Elroy-Stein, O. (2015) Proteomics-level analysis of myelin formation and regeneration in a mouse model for Vanishing White Matter disease. J Neurochem, 134, 513-526.
Geva, M., Cabilly, Y., Assaf, Y., Mindroul, N., Marom, L., Raini, G., Pinchasi, D. and Elroy-Stein, O. (2010) A mouse model for eukaryotic translation initiation factor 2B-leucodystrophy reveals abnormal development of brain white matter. Brain, 133, 2448-2461.
Gilad, Y., Nadassy, K. and Senderowitz, H. (2015). A reliable computational workflow for the selection of optimal screening libraries. J Cheminform 7, 61. DOI: 10.1186/s13321-015-0108-0.
Giorgi, C., De Stefani, D., Bononi, A., Rizzuto, R. and Pinton, P. (2009) Structural and functional link between the mitochondrial network and the endoplasmic reticulum. Int J Biochem Cell Biol, 41, 1817-1827.

Hayashi, T. and Su, T. P. (2007). Sigma-1 receptor chaperones at the ER-mitochondrion interface regulate Ca(2+) signaling and cell survival. Cell 131(3), 596-610. DOI: 10.1016/j.cell.2007 0.08.036.
Heiss, E. H., Kramer, M. P., Atanasov, A. G., Beres, H., Schachner, D. and Dirsch, V. M. (2014). Glycolytic switch in response to betulinic acid in non-cancer cells. PLoS One 9(12), e115683. DOI: 10.1371/journal.pone.0115683.
Horzinski, L., Kantor, L., Huyghe, A., Schiffmann, R., Elroy-Stein, O., Boespflug-Tanguy, O. and Fogli, A. (2010) Evaluation of the endoplasmic reticulum-stress response in eIF2B-mutated lymphocytes and lymphoblasts from CACH/VWM patients. BMC Neurol, 10, 94.
Ingham, P. W. and McMahon, A. P. (2001) Hedgehog signaling in animal development: paradigms and principles. Genes Dev. 15(23):3059-87.Kantor, L., Harding, H. P., Ron, D., Schiffmann, R., Kaneski, C. R., Kimball, S. R. and Elroy-Stein, O. (2005) Heightened stress response in primary fibroblasts expressing mutant eIF2B genes from CACH/VWM leukodystrophy patients. Hum Genet, 118, 99-106.
Kantor, L., Pinchasi, D., Mintz, M., Hathout, Y., Vanderver, A. and Elroy-Stein, O. (2008) A point mutation in translation initiation factor 2B leads to a continuous hyper stress state in oligodendroglial-derived cells. PLoS ONE, 3, e3783.
Kwon, I. K., S. C. Lee, Y. S. Hwang and J. S. Heo (2015). “Mitochondrial function contributes to oxysterol-induced osteogenic differentiation in mouse embryonic stem cells.” Biochim Biophys Acta 1853(3): 561-572.
Lan, Y., Chen, Y., Xu, X., Qiu, Y., Liu, S., Liu, X., et al. (2014). Synthesis and biological evaluation of a novel sigma-1 receptor antagonist based on 3,4-dihydro-2(1H)-quinolinone scaffold as a potential analgesic. Eur J Med Chem 79, 216-230. DOI: 10.1016/j.ejmech.2014.04.019.
Leegwater, P. A., Vermeulen, G., Konst, A. A. et al. (2001) Subunits of the translation initiation factor eIF2B are mutant in leukoencephalopathy with vanishing white matter. Nat Genet, 29, 383-388.
Li, D., Zhang, S. Z., Yao, Y. H., Xiang, Y., Ma, X. Y., Wei, X. L., et al. (2017). Sigma-1 receptor agonist increases axon outgrowth of hippocampal neurons via voltage-gated calcium ions channels. CNS Neurosci Ther 23(12), 930-939. DOI: 10.1111/cns.12768.Lisak, R. P., Nedelkoska, L. and Benjamins, J. A. (2014) Effects of dextromethorphan on glial cell function: proliferation, maturation, and protection from cytotoxic molecules. Glia, 62, 751-762.
Malhotra, A., A. Dey, N. Prasad and A. M. Kenney (2016). “Sonic Hedgehog Signaling Drives Mitochondrial Fragmentation by Suppressing Mitofusins in Cerebellar Granule Neuron Precursors and Medulloblastoma.” Mol Cancer Res 14(1): 114-124.
Marom, L., Ulitsky, I., Cabilly, Y., Shamir, R. and Elroy-Stein, O. (2011) A point mutation in translation initiation factor eIF2B leads to function—and time-specific changes in brain gene expression. PLoS One, 6, e26992.
Maurice, T. and Su, T. P. (2009). The pharmacology of sigma-1 receptors. Pharmacol Ther 124(2), 195-206. DOI: 10.1016/j.pharmthera.2009.07.001.
Mori, T., Hayashi, T., Hayashi, E. and Su, T. P. (2013). Sigma-1 receptor chaperone at the ER-mitochondrion interface mediates the mitochondrion-ER-nucleus signaling for cellular survival. PLoS One 8(10), e76941. DOI: 10.1371/journal.pone.0076941.
Nguyen, L., Lucke-Wold, B. P., Mookerjee, S., Kaushal, N. and Matsumoto, R. R. (2017). Sigma-1 Receptors and Neurodegenerative Diseases: Towards a Hypothesis of Sigma-1 Receptors as Amplifiers of Neurodegeneration and Neuroprotection. Adv Exp Med Biol 964, 133-152. DOI: 10.1007/978-3-319-50174-1_10.
Nury, T., Zarrouk, A., Mackrill, J. J., Samadi, M., Durand, P., Riedinger, J. M., et al. (2015). Induction of oxiapoptophagy on 158N murine oligodendrocytes treated by 7-ketocholesterol-, 7beta-hydroxycholesterol-, or 24(S)-hydroxycholesterol: Protective effects of alpha-tocopherol and docosahexaenoic acid (DHA; C22:6 n-3). Steroids 99(Pt B), 194-203. DOI: 10.1016/j.steroids.2015.02.003.
Raini, G., Sharet, R., Herrero, M., Atzmon, A., Shenoy, A., Geiger, T. and Elroy-Stein, O. (2017) Mutant eIF2B leads to impaired mitochondrial oxidative phosphorylation in Vanishing White Matter Disease. J Neurochem, March 17. doi: 10.1111/jnc.14024. [Epub ahead of print]
Ruscher, K. and Wieloch, T. (2015). The involvement of the sigma-1 receptor in neurodegeneration and neurorestoration. J Pharmacol Sci 127(1), 30-35. DOI: 10.1016/j.jphs.2014.11.011.
Ryan, M. T. and Hoogenraad, N. J. (2007) Mitochondrial-nuclear communications. Annu Rev Biochem, 76, 701-722.
Ryskamp, D., Wu, J., Geva, M., Kusko, R., Grossman, I., Hayden, M., et al. (2017). The sigma-1 receptor mediates the beneficial effects of pridopidine in a mouse model of Huntington disease. Neurobiol Dis 97(Pt A), 46-59. DOI: 10.1016/j.nbd.2016.10.006.
Sahlholm, K., Sijbesma, J. W., Maas, B., Kwizera, C., Marcellino, D., Ramakrishnan, N. K., et al. (2015). Pridopidine selectively occupies sigma-1 rather than dopamine D2 receptors at behaviorally active doses. Psychopharmacology (Berl) 232(18), 3443-3453. DOI: 10.1007/s00213-015-3997-8.
Samanta, J., E. M. Grund, H. M. Silva, J. J. Lafaille, G. Fishell and J. L. Salzer (2015). “Inhibition of Gli1 mobilizes endogenous neural stem cells for remyelination.” Nature 526(7573): 448-452.
Su, T. P., Su, T. C., Nakamura, Y. and Tsai, S. Y. (2016) The Sigma-1 Receptor as a Pluripotent Modulator in Living Systems. Trends Pharmacol Sci, 37, 262-278.
Schweizer, R. A. S., Atanasov, A. G., Frey, B. M. and Odermatt, A. (2003). A Rapid Screening Assay for Inhibitors of 11β-Hydroxysteroid Dehydrogenases (11β-Hsd): Flavanone Selectively Inhibits 11β-Hsd1 Reductase Activity. Molecular and Cellular Endocrinology 212(1): 41-49.DOI: www(dot)doi(dot)org/10101.(dot)j(dot)mce(dot)2003.09.027.
Solly, K., Mundt, S. S., Zokian, H. J., Ding, G. J.-F., Hermanowski-Vosatka, A., Strulovici, B., et al. (2005). High-Throughput Screening of 11β-Hydroxysteroid Dehydrogenase Type 1 in Scintillation Proximity Assay Format. ASSAY and Drug Development Technologies 3(4): 377-384.DOI: 10.1089/adt.2005.3.377.
Taipale, J., J. K. Chen, M. K. Cooper, B. Wang, R. K. Mann, L. Milenkovic, M. P. Scott and P. A. Beachy (2000). “Effects of oncogenic mutations in Smoothened and Patched can be reversed by cyclopamine.” Nature 406(6799): 1005-1009
van der Voorn, J. P., van Kollenburg, B., Bertrand, G., Van Haren, K., Scheper, G. C., Powers, J. M. and van der Knaap, M. S. (2005) The unfolded protein response in vanishing white matter disease. J Neuropathol Exp Neurol, 64, 770-775.
van Kollenburg, B., van Dijk, J., Garbern, J., Thomas, A. A., Scheper, G. C., Powers, J. M. and van der Knaap, M. S. (2006) Glia-specific activation of all pathways of the unfolded protein response in vanishing white matter disease. J Neuropathol Exp Neurol, 65, 707-715.
Verity, A. N. and Campagnoni, A. T. (1988). Regional expression of myelin protein genes in the developing mouse brain: in situ hybridization studies. J Neurosci Res 21(2-4), 238-248.
Wu, C. L., S. D. Chen, C. S. Hwang and D. I. Yang (2009). “Sonic hedgehog mediates BDNF-induced neuroprotection against mitochondrial inhibitor 3-nitropropionic acid.” Biochem Biophys Res Commun 385(1): 112-117.
Wu, G., Robertson, D. H., Brooks, C. L., 3rd and Vieth, M. (2003). Detailed analysis of grid-based molecular docking: A case study of CDOCKER-A CHARMm-based MD docking algorithm. J Comput Chem 24(13), 1549-1562. DOI: 10.1002/jcc.10306.
Wyrwoll, C. S., Holmes, M. C. and Seckl, J. R. (2011). 11beta-hydroxysteroid dehydrogenases and the brain: from zero to hero, a decade of progress. Front Neuroendocrinol 32(3), 265-286. DOI: 10.1016/j.yfrne.2010.12.001.
Yoshimura, K. and S. Takeda (2012). “Hedgehog signaling regulates myelination in the peripheral nervous system through primary cilia.” Differentiation 83(2): 578-85.Zhang, Y., Lv, X., Bai, Y. et al. (2015) Involvement of sigma-1 receptor in astrocyte activation induced by methamphetamine via up-regulation of its own expression. J Neuroinflammation, 12, 29.
Zhang, Y., Lv, X., Bai, Y., Zhu, X., Wu, X., Chao, J., et al. (2015). Involvement of sigma-1 receptor in astrocyte activation induced by methamphetamine via up-regulation of its own expression. J Neuroinflammation 12, 29. DOI: 10.1186/s12974-015-0250-7.

1. A method of treating leukodystrophy in a subject in need thereof, the method comprising administering to the subject a therapeutically effective amount of an agent capable of up-regulating activity and/or expression of a component participating in a Sigma-1 Receptor (Sig-1R) signaling pathway, thereby treating the leukodystrophy in the subject.

2. (canceled)

3. The method of claim 1, wherein said component is selected from the group consisting of Sig-1R, CYC1, PHB, SLC25A11, SLC25A39, VSAC2, BiP, IRE1, RAC1, VDAC2, IP3R, Ankyrin, Insig, Emerin, RanBP2, ELMOD, UP1, C14orf1, CYP51A1, CFTR, EIF5A, GANAB, HSD17B1, 2HSPA5, NSDHL, RDH11, RPN2, SC4MOL, SEC61A2, SQLE, SURF4, TM7SF2, NACA2, PDZD11, RAF1, RPS27A, SEC61A2, TM7SF2, UBA52, UBC, XPO1, XPOT, CLN3, LBR, NUP205 and RAE1.

4. The method of claim 1, wherein said component is Sig-1R.

5. (canceled)

6. The method of claim 1, wherein said agent is a small molecule.

7. The method of claim 6, wherein said small molecule is a compound represented by Formula I: 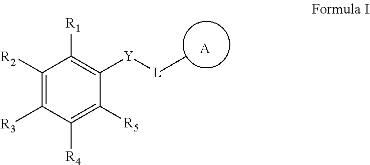
wherein:
R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;
Y is selected from O, S and NR′, wherein R′ is selected from hydrogen, alkyl, cycloalkyl, alkaryl, cycloalkyl and aryl;
L is a substituted or unsubstituted, saturated or unsaturated hydrocarbon chain of 2 to 10 carbon atoms in length, optionally interrupted by one or more heteroatoms selected from O, S and NR′; and
A is a heterocyclic moiety.

8. The method of claim 7, wherein said small molecule is selected from the group consisting of 4-[5-(3-methylphenoxy)pentyl]morpholine, 4-[5-(3,5-dimethylphenoxy)pentyl]morpholine, 4-[5-(3,4-dimethylphenoxy)pentyl]morpholine, 4-[6-(3-methylphenoxy)hexyl]morpholine, 4-[4-(3-methylphenoxy)butyl]morpholine, 4-[4-(3,4-dimethylphenoxy)butyl]morpholine, 4-[5-(3-methoxyphenoxy)pentyl]morpholine, 4-[5-(3-chlorophenoxy)pentyl]morpholine and 1-[5-(2-fluorophenoxy)pentyl]-4-methylpiperazine.

9. The method of claim 6, wherein said small molecule is selected from the group consisting of 4-[5-(3-methylphenoxy)pentyl]morpholine, Pre-084, pridopidine, dextromethorphan, SA4503, pentazocine, SKF-10047, 3-ppp, Fluvoxamine, Igmesine, Pregnenolone-S, DHEA-S, Donepezil, PPBP, Clorgyline, Fluoxetine, Imipramine, Sertaline, Carbetapentane, Dimemorfan, Amantadine, Memantine, Cocaine, BD 737, 4-IBP, OPC-14523, Anavex 2-73, Amitriptyline, L-687,384, Dimethyltryptamine, Methylphenylpiracetam and SOMCL-668.

10. The method of claim 6, wherein said small molecule is 4-[5-(3-methylphenoxy)pentyl]morpholine, Anavex, Pre-084 or pridopidine.

11-26. (canceled)

27. A method of treating a disease that can benefit from up-regulating activity of Sigma-1 Receptor (Sig-1R), the method comprising administering to the subject a therapeutically effective amount of:
(i) 4-[5-(3-methylphenoxy)pentyl]morpholine; or
(ii) a compound represented by Formula I: 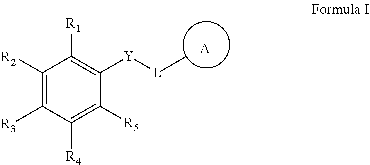
wherein:
R₁-R₅ are each independently selected from hydrogen, alkyl, alkenyl, alkynyl, cycloalkyl, aryl, heteroalicyclic, heteroaryl, halo, hydroxy, thiol, alkoxy, thioalkoxy, aryloxy, thioaryloxy, alkaryl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide;
Y is selected from O, S and NR′, wherein R′ is selected from hydrogen, alkyl, cycloalkyl, alkaryl, cycloalkyl and aryl;
L is a substituted or unsubstituted, saturated or unsaturated hydrocarbon chain of 2 to 10 carbon atoms in length, optionally interrupted by one or more heteroatoms selected from O, S and NR′; and
A is a heterocyclic moiety,
thereby treating the disease in the subject.

28-30. (canceled)

31. The method of claim 27, wherein said compound of (ii) is selected from the group consisting of 4-[5-(3-methylphenoxy)pentyl]morpholine, 4-[5-(3,5-dimethylphenoxy)pentyl]morpholine, 4-[5-(3,4-dimethylphenoxy)pentyl]morpholine, 4-[6-(3-methylphenoxy)hexyl]morpholine, 4-[4-(3-methylphenoxy)butyl]morpholine, 4-[4-(3,4-dimethylphenoxy)butyl]morpholine, 4-[5-(3-methoxyphenoxy)pentyl]morpholine, 4-[5-(3-chlorophenoxy)pentyl]morpholine and 1-[5-(2-fluorophenoxy)pentyl]-4-methylpiperazine.

32. A method of treating a disease that can benefit from down-regulating activity of sonic hedgehog (SHH) signaling pathway, the method comprising administering to the subject a therapeutically effective amount of a compound selected from the group consisting of 1-allyl-2-(3,4,5-trimethoxyphenyl)-1H-benzimidazole and 1-(2-fluorophenyl)-4-(phenylacetyl)piperazine, thereby treating the disease in the subject.

33. (canceled)

34. A method of treating a disease that can benefit from up-regulating activity of sonic hedgehog (SHH) signaling pathway, the method comprising administering to the subject a therapeutically effective amount of 1-allyl-2-(2-phenylvinyl)-1H-benzimidazole, thereby treating the disease in the subject.

35. (canceled)

36. The method of claim 34, wherein said disease is selected from the group consisting of skeletal muscle regeneration following injury and brain recovery following ischemic stroke.

37. The method of claim 27, wherein said disease is associated with mitochondrial dysfunction, oxidative stress and/or ER stress.

38. A method of treating a disease associated with mitochondrial dysfunction, oxidative stress and/or ER stress, the method comprising administering to the subject a therapeutically effective amount of a compound selected from the group consisting of 5-benzyl-2-[(2-chlorophenyl)imino]-1,3-thiazolidin-4-one, 5-butyl-3-{[2-(4-morpholinyl)ethyl]thio}-5H-[1,2,4]triazino[5,6-b]indole, 2-phenyl-N′-({5-[3-(trifluoromethyl)phenyl]-2-furyl}methylene)acetohydrazide, 1-{3-[(4-chlorobenzyl)oxy]phenyl}ethanone, 1-allyl-2-(3,4,5-trimethoxyphenyl)-1H-benzimidazole, 7-(difluoromethyl)-N-[2-(4-morpholinyl)ethyl]-5-phenylpyrazolo[1,5-a]pyrimidine-3-carboxamide, 1-phenyl-4-[4-(2-thienylcarbonyl)-1-piperazinyl]phthalazine, 4-[5-(3-methylphenoxy)pentyl]morpholine, 1-(2-fluorophenyl)-4-(phenylacetyl)piperazine, 2-{[2-oxo-2-(1-piperidinyl)ethyl]thio}-4-phenyl-6-(trifluoromethyl)pyrimidine, N-[2-(phenylthio)cyclohexyl]benzenesulfonamide, 1-{[3-(benzyloxy)phenyl]carbonothioyl}-4-methylpiperazine, 5-phenyl-N-(2-thienylmethyl)-7-(trifluoromethyl)pyrazolo[1,5-a]pyrimidine-2-carboxamide, 2-(2,3-dihydro-9H-imidazo[1,2-a]benzimidazol-9-yl)-1-(4-hydroxyphenyl)ethanone hydrobromide, 2-[(2,5-dimethoxyphenyl)diazenyl]-1-methyl-1H-benzimidazole, 2-[(2,6-dimethyl-1-piperidinyl)carbonyl]-7-methyl-5-phenylpyrazolo[1,5-a]pyrimidine, 2-(4-morpholinylmethyl)-1-(1-naphthylmethyl)-1H-benzimidazole, 5-isopropyl-N-methyl-3-phenylpyrazolo[1,5-a]pyrimidin-7-amine, 4-[5-(3,5-dimethylphenoxy)pentyl]morpholine, 4-[5-(3,4-dimethylphenoxy)pentyl]morpholine, 4-[6-(3-methylphenoxy)hexyl]morpholine, 4-[4-(3-methylphenoxy)butyl]morpholine, 4-[4-(3,4-dimethylphenoxy)butyl]morpholine, 4-[5-(3-methoxyphenoxy)pentyl]morpholine, 4-[5-(3-chlorophenoxy)pentyl]morpholine and 1-[5-(2-fluorophenoxy)pentyl]-4-methylpiperazine, thereby treating the disease in the subject.

39. (canceled)

40. The method of claim 27, wherein said disease is selected from the group consisting of leukodystrophy, multiple sclerosis, cancer, OXPHOS diseases, lactic acidosis and stroke-like episodes (MELAS), myoclonus epilepsy with ragged red fibers (MERRF), deafness-dystonia syndrome (DDP), Parkinson disease, diabetes mellitus and sensorineural hearing impairment.

41. The method of claim 40, wherein said cancer is selected from the group consisting of lung cancer, stomach cancer, esophagus cancer, pancreas cancer, prostate cancer, breast cancer, liver cancer, brain cancer, medulloblastoma, Basal cell carcinoma (BCC), cancer stem cells, rhabdomyosarcomas, glioma, multiple myeloma and chronic myelogenous leukemia (CML).

42. The method of claim 27, wherein said disease is leukodystrophy.

43. The method of claim 1, wherein said leukodystrophy is selected from the group consisting of vanishing white matter (VWM) disease, Krabbe disease, Metachromatic leukodystrophy, Pelizaeus-Merzbacher disease, Canavan disease, Adrenoleukodystrophy, Adrenomyeloneuropathy, Alexander disease, Cerebrotendineous xanthomatosis and Refsum disease.

44. The method of claim 1, wherein said leukodystrophy is vanishing white matter (VWM) disease.

45-47. (canceled)


SEQUENCE LISTING (4 sequences)

SEQ ID NO: 1 - DNA, 24 bp, Artificial Sequence
  Single strand DNA oligonucleotide
cccatagggtctcgggtctcaaac

SEQ ID NO: 2 - DNA, 25 bp, Artificial Sequence
  Single strand DNA oligonucleotide
ggaggacctgcggctgactgtgtaa

SEQ ID NO: 3 - DNA, 22 bp, Artificial Sequence
  Single strand DNA oligonucleotide
tggcaaagtggagattgttgcc

SEQ ID NO: 4 - DNA, 21 bp, Artificial Sequence
  Single strand DNA oligonucleotide
aagatggtgatgggcttcccg